SEQUENCE LISTING (19 sequences)

SEQ ID NO: 1 - protein, 379 aa, Homo sapiens
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 2 - DNA, 21 bp, synthetic construct
  source 1..21: mol_type = other DNA
gtggcattcaaggagtacctc

SEQ ID NO: 3 - DNA, 21 bp, synthetic construct
  source 1..21: mol_type = other DNA
cagattggctacccaactgtt

SEQ ID NO: 4 - DNA, 21 bp, synthetic construct
  source 1..21: mol_type = other DNA
ccaagtgctgccgtcattttc

SEQ ID NO: 5 - DNA, 22 bp, synthetic construct
  source 1..22: mol_type = other DNA
agattcgggatatgctgttggc

SEQ ID NO: 6 - DNA, 22 bp, synthetic construct
  source 1..22: mol_type = other DNA
tgatggccttcgattctggatt

SEQ ID NO: 7 - DNA, 21 bp, synthetic construct
  source 1..21: mol_type = other DNA
ggaaggtgtaatccgtctcca

SEQ ID NO: 8 - DNA, 21 bp, synthetic construct
  source 1..21: mol_type = other DNA
ggctcgcagggatgatttcaa

SEQ ID NO: 9 - DNA, 21 bp, synthetic construct
  source 1..21: mol_type = other DNA
tcgggtcctagaccagtgttc

SEQ ID NO: 10 - DNA, 20 bp, synthetic construct
  source 1..20: mol_type = other DNA
atcgtttccgcttaacggcg

SEQ ID NO: 11 - DNA, 20 bp, synthetic construct
  source 1..20: mol_type = other DNA
cttcaaggtggacatctggt

SEQ ID NO: 12 - DNA, 20 bp, synthetic construct
  source 1..20: mol_type = other DNA
aagtgcgactccgcgttcag

SEQ ID NO: 13 - DNA, 20 bp, synthetic construct
  source 1..20: mol_type = other DNA
ggtaccggggcagctactgg

SEQ ID NO: 14 - DNA, 20 bp, synthetic construct
  source 1..20: mol_type = other DNA
gatctaatgttaaagactgg

SEQ ID NO: 15 - DNA, 20 bp, synthetic construct
  source 1..20: mol_type = other DNA
tgctggcaccagaacgaatg

SEQ ID NO: 16 - DNA, 20 bp, synthetic construct
  source 1..20: mol_type = other DNA
aagagtaagttatttgacgt

SEQ ID NO: 17 - DNA, 20 bp, synthetic construct
  source 1..20: mol_type = other DNA
acgtgtaaggcgaacgcctt

SEQ ID NO: 18 - DNA, 20 bp, synthetic construct
  source 1..20: mol_type = other DNA
cagtagtccaagttcgtgcg

SEQ ID NO: 19 - DNA, 20 bp, synthetic construct
  source 1..20: mol_type = other DNA
tcatcggcaagtacctgatg

(19) United States
(12) Patent Application Publication
(10) Pub. No.: US 2025/0295686 A1
(43) Pub. Date: Sep. 25, 2025

(54) COMBINATION DECITABINE AND MPS1 INHIBITOR THERAPY TO PRIME CANCER IMMUNOGENICITY

(71) Applicant: Dana-Farber Cancer Institute, Inc., Boston, MA (US)

(72) Inventors: David Barbie, Andover, MA (US); Shunsuke Kitajima, Allston, MA (US); Tetsuo Tani, Brookline, MA (US); Cloud P. Paweletz, Boston, MA (US); Prafulla C. Gokhale, Lexington, MA (US)

(21) Appl. No.: 18/702,726

(22) PCT Filed: Oct. 18, 2022
(86) PCT No.: PCT/US2022/078291

§ 371 Date: Apr. 18, 2024

Related U.S. Application Data
(60) Provisional application No. 63/256,978, filed on Oct. 18, 2021.

(51) Int. Cl. A61K 31/706; A61K 31/5025; A61P 35/00

(52) U.S. Cl. CPC A61K 31/706; A61K 31/5025; A61P 35/00

(57) ABSTRACT

Provided herein are methods for treating a subject having cancer comprising administering to the subject a therapeutically effective amount of an epigenetic inhibitor, followed by a therapeutically effective amount of a DNA damaging agent. Also provided herein are methods for treating a subject having cancer based on the expression levels of STING and/or an epigenetic regulatory enzyme in a biological sample taken from the subject. Further provided herein are kits comprising an epigenetic inhibitor and a DNA damaging agent.

### RELATED APPLICATIONS

This application claims priority under 35 U.S.C. § 119(e) to U.S. Provisional Application, U.S.S.N., 63/256,978, filed Oct. 18, 2021, the contents of which is incorporated herein by reference.

### FEDERALLY SPONSORED RESEARCH

This invention was made with government support under grant number R01 CA190394, awarded by the National Institutes of Health (NIH). The government has certain rights in the invention.

### REFERENCE TO AN ELECTRONIC SEQUENCE LISTING

The contents of the electronic sequence listing (D050470200W000-SEQ-AZW.xml; Size: 18,161 bytes; and Date of Creation: Oct. 17, 2022) is herein incorporated by reference in its entirety.

### BACKGROUND OF THE INVENTION

Despite the impressive efficacy of PD-(L)1 blockade in lung cancer, specific genomic subsets promote intrinsic resistance. Co-mutation of the STK11/LKB1 tumor suppressor with oncogenic KRAS constitutes a dominant resistant phenotype. KRAS-LKB1 (KL) mutant lung cancers silence STING due to intrinsic mitochondrial dysfunction, resulting in T cell exclusion and resistance to PD-(L)1 blockade. As described herein, KL cells also minimize intracellular accumulation of 2′3′-cGAMP to further avoid downstream STING and STAT1 activation. Because KL tumors epigenetically silence STING, but largely retain cyclic GMP-AMP synthase (cGAS) expression, they remain vulnerable to DNA damaging strategies that could restore intrinsic immunogenicity. Accordingly, novel therapeutic strategies are needed to expand immunotherapy benefits against KL mutant lung cancers.

### SUMMARY OF THE INVENTION

The present disclosure is based on the unexpected discovery that transient MPS1 inhibition potently re-engages the STING pathway in KL cells via micronuclei generation. This effect is markedly amplified by epigenetic de-repression of STING and only requires pulse MPS1 inhibitor treatment, which creates a therapeutic window compared to non-dividing cells. As described herein, a single course of treatment with an epigenetic inhibitor (e.g., decitabine) followed by pulse treatment with a DNA-damaging agent (e.g., an MPS1 inhibitor such as BAY-1217389) restores T cell infiltration, enhances anti-PD1 efficacy, and results in durable response in vivo, without evidence of significant toxicity. This sequential therapeutic approach reverses STING silencing and compels cancer cells to sense micronuclei, which are potent activators of cyclic GMP-AMP synthase (cGAS). This strategy primarily impacts dividing cells and targets a major underlying mechanism of KL tumor escape using pulse scheduling of inhibitors undergoing clinical development.

Accordingly, in one aspect, the present disclosure provides methods of treating a subject having cancer, comprising (i) administering to the subject a therapeutically effective amount of an epigenetic inhibitor; and (ii) administering to the subject a therapeutically effective amount of a DNA damaging agent.

In another aspect, the present disclosure provides methods of treating a subject having cancer comprising (i) obtaining a biological sample from the subject having cancer; (ii) determining the level of expression in the biological sample of STING, an epigenetic regulatory enzyme, or both STING and an epigenetic regulatory enzyme; and (iii) administering a treatment to the subject if the biological sample comprises low levels of STING expression, high levels of expression of an epigenetic regulatory enzyme, or both low levels of STING expression and high levels of expression of an epigenetic regulatory enzyme. In some embodiments, the treatment comprises: (i) administering to the subject a therapeutically effective amount of an epigenetic inhibitor; and (ii) administering to the subject a therapeutically effective amount of a DNA damaging agent.

In various embodiments of any of the methods disclosed herein, the epigenetic inhibitor may increase or restore expression of STING. In some embodiments, the epigenetic inhibitor inhibits DNA methylation, histone methylation, or histone deacetylation. In some embodiments, the epigenetic inhibitor comprises a DMNT1 inhibitor, an EZH2 inhibitor, or an HDAC inhibitor. In some embodiments, the epigenetic inhibitor comprises a DMNT1 inhibitor. In certain embodiments, the epigenetic inhibitor comprises decitabine.

In some embodiments, the DNA damaging agent used in any of the methods disclosed herein comprises an agent that induces the formation of micronuclei. In some embodiments, the DNA damaging agent comprises an MPS1 inhibitor or an anti-folate drug. In some embodiments, the DNA damaging agent comprises an MPS1 inhibitor. In certain embodiments, the DNA damaging agent comprises BAY-1217389.

In some embodiments, the cancer treated using any of the methods disclosed herein is lung cancer. In some embodiments, the cancer is non-small cell lung cancer. In certain embodiments, the cancer is a KRAS-LKB1 (KL) mutant cancer.

In some embodiments, the step of administering the epigenetic inhibitor is performed prior to the step of administering the DNA damaging agent. In some embodiments, the epigenetic inhibitor is administered to the subject for about 3, about 4, about 5, about 6, about 7, about 8, about 9, about 10, about 11, or about 12 or more days. In certain embodiments, the epigenetic inhibitor is administered to the subject for 7 days. In some embodiments, the DNA damaging agent is administered to the subject for about 1, about 2, about 3, or about 4 or more days. In certain embodiments, the DNA damaging agent is administered to the subject for 2 days.

In some embodiments, any of the methods disclosed herein may further comprise comprising administering a therapeutically effective amount of the DNA damaging agent to the subject an additional time. In certain embodiments, the DNA damaging agent is administered to the subject an additional time about two weeks after the DNA damaging agent is administered to the subject a first time.

In yet another aspect, the present disclosure provides kits comprising an epigenetic inhibitor and a DNA damaging agent. In some embodiments, the kit further comprises at least one pharmaceutically acceptable excipient.

In some embodiments, the epigenetic inhibitor increases or restores expression of STING. In some embodiments, the epigenetic inhibitor inhibits DNA methylation, histone methylation, or histone deacetylation. In some embodiments, the epigenetic inhibitor comprises a DMNT1 inhibitor, an EZH2 inhibitor, or an HDAC inhibitor. In some embodiments, the epigenetic inhibitor comprises a DMNT1 inhibitor. In certain embodiments, the epigenetic inhibitor comprises decitabine.

In some embodiments, the DNA damaging agent comprises an agent that induces the formation of micronuclei. In some embodiments, the DNA damaging agent comprises an MPS1 inhibitor or an anti-folate drug. In some embodiments, the DNA damaging agent comprises an MPS1 inhibitor. In certain embodiments, the DNA damaging agent comprises BAY-1217389.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures, and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1I show that KL cells exhibit low tolerability to accumulation of intracellular 2′3′-cGAMP. FIGS. 1A and 1B show ELISA results of human CXCL10 levels in conditioned medium (CM) derived from NSCLC cells treated with ±3.125, 6.25, 12.5, 25, 50, or 100 μM 2′3′-cGAMP or ADU-S100 for 24 hours. H2122, H1355, H23, and HCC44 KL cell lines have a p53 mutation. FIG. 1C shows ELISA results of human CXCL10 or IFN-β levels in CM derived from KL cells (H2122, H1944, H1355) or KP cells (H2009, H441, H358, H1792) transduced with the indicated vectors (Luciferase control, left; cGAS, right). FIG. 1D provides an immunoblot (“IB”) of the indicated proteins in KL or KP cells transduced with the indicated vectors. FIG. 1E shows ELISA results of intracellular 2′3′-cGAMP levels in KL cells (H2122, H1944, H1355) or KP cells (H2009, H441, H358, H1792) transduced with the indicated vectors (Luciferase control, left; cGAS, right). FIG. 1F shows the total cell number of H1944 cells transduced with the indicated vectors at each measuring point (day 0, day 3, day 8, day 13, or day 18). 3×10⁵ cells were plated onto a 6-well plate at day 0. FIG. 1G shows IBs of the indicated proteins, and FIGS. 1H and 1I show ELISA results of human CXCL10 in CM (FIG. 1H) or intracellular 2′3′-cGAMP levels (FIG. 1I) in H1944 cells transduced with the indicated vectors (scramble sgRNA, left; STAT1 sgRNA, right). p-values were calculated by unpaired two-tailed Student's t test (FIGS. 1C, 1E, and 1F), one-way ANOVA followed by Dunnet's post-hoc test (FIGS. 1A and 1B), or two-way ANOVA followed by Sidak's post-hoc test (FIGS. 1H and 1I), *p<0.05, **p<0.01.

FIGS. 2A-2K provide data showing that KL cells exhibit low tolerability to accumulation of intracellular 2′3′-cGAMP. FIG. 2A provides an IB of the indicated proteins in KL cells (H2122, H1944, H1355, A549, H23, A427) or KP cells (H2009, H358, H1792). FIG. 2B provides an IB of the indicated proteins in KL cells (H2122, H1944, H1355, H647, A549, H23, A427, HCC44) or KP cells (H2009, H441, H358, H1792). KL cell lines with an asterisk contain a p53 mutation. FIG. 2C shows ELISA results of human CXCL10 in conditioned medium (CM) in H1944, H2009, HUVEC, or THP1 cells treated with 5 μg/ml 2′3′-cGAMP (cGAMP) or 25 μM ADU-S100 (ADU) for 24 hours (control, left; cGAMP or ADU, right). THP1 cells were differentiated to macrophages in the presence of 25 nM PMA for 48 hours. FIGS. 2D-2E show IBs of the indicated proteins in KL (H2122, H1944, and H1355) or KP (H2009 and H441) cells transduced with the indicated vectors. FIG. 2F shows an IB of the indicated proteins, and FIGS. 2G and 2H show ELISA results of human CXCL10 in CM (FIG. 2G) or intracellular 2′3′-cGAMP levels (FIG. 2H) in H1944 cells transduced with the indicated vectors. FIGS. 2I-2J show the total cell number of H1944 cells treated with 1 μM ruxolitinib (Ruxo) at each measuring point (day 0, day 3, day 8, day 13, or day 18) (FIG. 2I), or H2122 or H1355 cells transduced with the indicated vectors at each time point (day 0, day 3, day 5, day 8, day 12, or day 17) (FIG. 2J). 3×10⁵ cells were plated onto a 6-well plate at day 0. FIG. 2K shows an IB of the indicated proteins in KL cells transduced with the indicated vectors. p-values were calculated by unpaired two-tailed Student's t test (FIGS. 2C-2I, 2J), or one-way ANOVA followed by Tukey's post-hoc test (FIGS. 2G-2H), *p<0.05, **p<0.01.

FIGS. 3A-3L show screening of DNA-damaging agents to extract the drugs activating the STING pathway in KL cells. FIG. 3A shows relative RPKM values of cGAS in KL and KP cells from CCLE. FIG. 3B shows the schedule of drug treatment for the screening. GM: growth medium. CM: conditioned medium. FIG. 3C shows intracellular 2′3′-cGAMP levels in H2122 or H1944 cells treated with 0.5 μg/ml poly (dA:dT). FIGS. 3D and 3E show ELISA results of human CXCL10 in CM derived from H1944 (FIG. 3D) or H2122 (FIG. 3E) cells treated with the indicated DNA-damaging agents in accordance with the schedule for the screening. FIG. 3F shows ELISA results of human CXCL10 or IFN-β levels in CM derived from H1944 cells transduced with the indicated vectors, treated with 200 nM CFI-402257 in accordance with the schedule for the screening (scramble sgRNA (A), left; cGAS sgRNA (B), right). FIGS. 3G and 3H show IBs of the indicated proteins (FIG. 3G), or intracellular 2′3′-cGAMP levels (FIG. 3H), in H1944 cells transduced with the indicated vectors and treated with the indicated DNA-damaging agents in accordance with the schedule for the screening (FIG. 3H—scramble sgRNA (A), left; cGAS sgRNA (B), right). PEM: pemetrexed, MTX: methotrexate, CDDP: cisplatin, DTX: docetaxel, ETP: etoposide Prexa: prexasertib, Bara: barasertib, CFI: CFI-402257. FIGS. 3I, 3K, and 3L show IBs of the indicated proteins in H1944 cells transduced with the indicated vectors and treated with 200 nM CFI-402257, 100 nM BAY-1217389, or 250 nM CC-671 in accordance with the schedule for the screening. FIG. 3J shows ELISA results of human CXCL10 levels in CM derived from H1944 cells transduced with the indicated vectors and treated with 100 nM BAY-1217389 (Luciferase control (A), left; STING (B), right). p-values were calculated by unpaired two-tailed Student's t test (FIGS. 3A, 3C, and 3H), or two-way ANOVA followed by Tukey's post-hoc test (FIG. 3F, 3J), **p<0.01.

FIGS. 4A-4N provide data showing screening of DNA-damaging agents to extract the drugs activating the STING pathway in KL cells. FIG. 4A shows the percent inhibition in H1944 cells at each concentration of each DNA-damaging agent for 72 hours. The IC₅₀ value of each DNA-damaging agent is shown. FIG. 4B shows the ratio of propidium iodide (PI) positive cells in H1944 cells treated with the indicated DNA-damaging agents for 48 hours. FIG. 4C shows an IB of the indicated proteins in H1944 cells treated with the indicated DNA-damaging agents for 48 hours. FIG. 4D shows ELISA results of human CXCL10 or IFN-β levels in CM in H647 or H2122 cells transduced with the indicated vectors, and FIG. 4E shows an IB of the indicated proteins in H647 cells transduced with the indicated vectors, and treated with 100 nM BAY-1217389 (FIG. 4D—DMSO (A), left; BAY-1217389 (B), right). n.s., not significant. FIG. 4F shows an ELISA of human CXCL10 levels in CM, and FIG. 4G shows an IB of the indicated proteins in H1944 cells transduced with the indicated sgRNAs or vectors (FIG. 4F—scramble sgRNA (A), left; cGAS sgRNA (B), right). FIG. 4H shows an IB of the indicated proteins in A549 or H23 cells transduced with the indicated vectors and treated with 100 nM BAY-1217389. FIG. 4I shows the percent inhibition in KL cells transduced with the indicated vectors at each concentration of BAY-1217389 for 96 hours. FIGS. 4J and 4L show the schematic of cell growth analysis following pulse treatment with an MPS1 inhibitor—10 nM BAY-1217389 (FIG. 4J, upper). Phase contrast images (left) or total cell number (right) of H1944, H1355, or H647 cells transduced with the indicated vectors at day 21 (H1944), day 19 (H1355), or day 14 (H647) (FIG. 4J, lower right—scramble sgRNA (A), left; STAT1 sgRNA (B), right; FIG. 4L, right —scramble sgRNA (A), left; IFNAR1 sgRNA (B), right). FIG. 4K shows the percent inhibition in KP or KL cells at each concentration of BAY-1217389 for 96 hours. FIG. 4M shows ELISA results of human CXCL10 or IFN-β levels in CM in H1944 or H647 cells transduced with the indicated vectors (scramble sgRNA (A), left; IFNAR1 sgRNA (B), right), and FIG. 4N shows an IB of the indicated proteins in H647 cells transduced with the indicated vectors, and treated with 100 nM BAY-1217389. p-values were calculated by unpaired two-tailed Student's t test (FIGS. 4J, 4L), or two-way ANOVA followed by Sidak's post-hoc test (FIGS. 4D, 4G, 4M), **p<0.01.

FIGS. 5A-5H show that MPS1 inhibition induces micronuclei formation and subsequent STING activation in KL cells. FIG. 5A provides representative confocal microscope images of DAPI-staining in H1944 cells treated with 200 nM CFI-402257, 5 nM docetaxel, or 200 nM barasertib in accordance with the schedule for the screening. Arrows indicate micronuclei. Inset highlights a micronucleus. Scale bars: 10 μm. FIG. 5B shows the number of micronuclei in H1944 cells treated with the indicated DNA-damaging agents in accordance with the schedule for the screening. FIG. 5C shows relative mRNA expression of CXCL10 (y-axis) versus the number of micronuclei (x-axis) in H1944 cells treated with the indicated DNA-damaging agents in accordance with the schedule for the screening. R2 values and p-values for the correlation (Pearson's r correlation) are shown. FIG. 5D shows the quantification of cell cycle analysis through propidium iodide staining for the cells after treatment with 200 nM CFI-402257 (CFI), 2.5 μM cisplatin (CDDP), 5 μM etoposide (ETP), 500 nM pemetrexed (PEM), or 50 μM hydroxyurea (HU) for 48 hours. FIG. 5E shows ELISA results of human CXCL10 or IFN-β levels in CM, and FIG. 5F shows an IB of the indicated proteins in H1944 cells treated with 200 nM CFI-402257 in accordance with the indicated schedule. GM: growth medium. FIG. 5G show ELISA results of human CXCL10 in CM, and FIG. 5H shows an IB of the indicated proteins in H1944 or THP1 cells treated with 200 nM CFI-402257 in accordance with the schedule for the screening, or 10 μM ADU-S100 for 24 hours. THP1 cells were differentiated to macrophages in the presence of 25 nM PMA for 48 hours. p-values were calculated by one-way ANOVA followed by Tukey's post-hoc test (FIGS. 5E and 5G), **p<0.01.

FIGS. 6A-6T show the effects of Lamin B2 over-expression. FIG. 6A shows an IB of the indicated proteins in H1944 cells treated with the indicated vectors. FIG. 6B shows ELISA results of human CXCL10 levels in CM derived from H1944 cells transduced with the indicated vectors and treated with BAY-1217389 at the indicated concentration (Luciferase control (A), left; LaminB2 (B), right). FIG. 6C shows ELISA results of human CXCL10 levels in CM in H1944 or H647 cells transduced with the indicated vectors, and FIG. 6D shows an IB of the indicated proteins in H647 cells transduced with the indicated vectors, and treated with 200 nM barasertib or 5 nM docetaxel (FIG. 6C—scramble sgRNA (A), left; cGAS sgRNA (B), right). FIG. 6E shows ELISA results of human CXCL10 in CM in H1944 cells treated with docetaxel or barasertib at the indicated concentration in accordance with the schedule for the screening. FIG. 6F shows ELISA results of human CXCL10 or IFN-β levels in CM, and FIG. 6G shows an IB of the indicated proteins in H1944 cells transduced with the indicated vectors, and treated with 100 nM BAY-1217389 or 25 μM ADU (FIG. 6F—Luciferase control (A), left; LKB1 (B), middle; and LKB1-KD (C), right). FIG. 6H shows the quantification of the number of cGAS foci co-localized with micronuclei in H1944 cells transduced with the indicated vectors and treated with 100 nM BAY-1217389 (Luciferase control (A), left; LKB1 (B), right). FIG. 6I shows the ratio of cGAS-positive micronuclei relative to total number of micronuclei in H1944 cells transduced with the indicated vectors. n.s., not significant. FIG. 6J shows an IB of the indicated proteins in H1944 cells treated with the indicated vectors. FIG. 6K shows the quantification of cell cycle analysis through propidium iodide staining for H1944 cells transduced with the indicated vectors. FIG. 6L shows the total cell number of H1944 cells transduced with the indicated vectors at each measuring point (day 0, day 3, day 7, or day 11). FIG. 6M shows ELISA result of human CXCL10 levels in CM derived from KL cells (H2122, H1944, H647, A549, H23) or KP cells (H2009, H1792, H441, H358) treated with 100 nM BAY-1217389 (DMSO (A), left; BAY-1217389 (B), right). FIG. 6N shows representative confocal microscope images and FIG. 6O shows quantification of the number of cGAS foci co-localized with micronuclei in KP cells (H2009, H1792, H441, H358) or KL cells (H1944, H647, A549, H23) treated with 100 nM BAY-1217389 (FIG. 6O—DMSO (A), left; BAY-1217389 (B), right). FIG. 6P shows ELISA results of human CXCL10 levels in CM derived from LKB1 mutated cells (H1944, H1395, H838, H1568, H1568, H1437, H1755) or LKB1 wild type cells (H2228, H2087, H1793) treated with 100 nM BAY-1217389, and FIG. 6Q shows ELISA results with 0, 10, 25, 50, or 100 μM 2′3′-cGAMP for 24 hours (FIG. 6P—DMSO (A), left; BAY-1217389 (B), right). H1944 cells were treated with 100 μM 2′3′-cGAMP for 24 hours. H1944 cells with an asterisk contain KRAS mutation. FIG. 6R shows an IB of the indicated proteins in LKB1 mutated cells (A549, H1944, H1395, H838, H1568, H1437, H1755) or LKB1 wild type cells (H2228, H2087, H1793, HCC827, H2009). Cell lines with an asterisk contain KRAS mutation. FIG. 6S shows ELISA results of human CXCL10 in CM, and FIG. 6T shows an IB of the indicated proteins in H2009 or H358 cells transduced with the indicated vectors and treated with 100 nM BAY-1217389 (FIG. 3S—scramble sgRNA (A), left; ATG5 sgRNA (B), right). ELISA results also obtained for IFN-β in CM. p-values were calculated by unpaired two-tailed Student's t test (FIGS. 61, 6K, 6L, 6O), one-way ANOVA followed by Dunnet's post-hoc test (FIG. 6Q), or two-way ANOVA followed by Sidak's post-hoc test (FIG. 6C, 6F, 6H, 6S), *p<0.05, **p<0.01.

FIGS. 7A-7I show that combination treatment with MPS1 and epigenetic inhibitors cooperatively activates the STING pathway. FIG. 7A shows ELISA results of human CXCL10 or IFN-β levels in CM, and FIGS. 7B and 7C show IBs of the indicated proteins in H1944 transduced with the indicated vectors and treated with the indicated drugs (5 μM GSK, and/or 200 nM CFI) in accordance with pretreatment schedule (see FIG. 8B) (FIG. 7A—DMSO (A), left; GSK (B), right). FIG. 7D shows fluorescent images and FIG. 7E shows quantification of STING foci-containing cells (Arrows) of H1944 cells treated with the indicated drugs (5 μM GSK and/or 200 nM CFI). Scale bar 10 μM. FIGS. 7F-7H show ELISA results of human CXCL10 or IFN-β levels in CM derived from A549, H23, or A427 transduced with the indicated vectors and treated with the indicated drugs (50 nM DAC (FIG. 7F) or 100 nM DAC (FIGS. 7G and 7H), 5 μM GSK, and/or 200 nM CFI) in accordance with the pretreatment schedule (DMSO (A), left; DAC (B), center left; GSK (C), center right; DAC+GSK (D), right). FIG. 7I provides a schematic of the concept of sequential combination therapy with epigenetic inhibitors and MPS1 inhibitors. p-values were calculated by one-way (FIG. 7E) followed by Tukey's post-hoc test, or two-way (FIGS. 7A, 7F, 7G, and 7H) ANOVA followed by Sidak's post-hoc test, **p<0.01.

FIGS. 8A-8E show that combination treatment with MPS1 and epigenetic inhibitors cooperatively activates the STING pathway. FIG. 8A shows ELISA results of human CXCL10 levels in CM derived from KL cells (A549, H23, A427, H1944) or KP cells (H2009, H441, H358, H1792) treated with 200 nM CFI-402257 in accordance with the schedule for the screening (Control (A), left; CFI-402257 (B), right). FIG. 8B provides a schematic of pre-treatment with epigenetic inhibitors (50 nM decitabine (DAC) and/or 5 μM GSK126 (GSK)) and/or MPS1 inhibitor (200 nM CFI-402257 or 100 nM BAY-1217389). FIG. 8C shows an IB of the indicated proteins in KL cells (A549, H1355, H1944, H2122) treated with 100 nM decitabine (DAC) and/or 5 μM GSK126 (GSK) for 5 days. The lysates derived from KP cells (H2009, H441) are used as a positive control for STING expression in IB. p-values were calculated by unpaired two-tailed Student's t test (FIG. 8A), **p<0.01. FIG. 8D shows ELISA results of human CXCL10 or IFN-β levels in CM, and FIG. 8E show an IB of the indicated proteins in H1355 cells transduced with the indicated vectors and treated with the indicated drugs (5 μM GSK, and/or 100 nM BAY-1217389) in accordance with the pretreatment schedule (see FIG. 8B) (FIG. 8E—DMSO (A), left; GSK, right). P-values were calculated by two-way ANOVA followed by Sidak's post-hoc test (FIG. 8D), **p<0.01.

FIGS. 9A-9Q show that MPS1 inhibition upregulates human leukocyte antigens (HLAs) expression and immune infiltration into the peri-tumor region. FIGS. 9A and 9B show HLA-A.B.C (FIG. 9A) or PD-L1 (FIG. 9B) expression on the cell surface in H1944 cells transduced with the indicated vectors and treated with the indicated drugs (200 nM CFI, or 25 μM ADU) (DMSO (A); CFI (B); and ADU (C)). FIGS. 9C and 9D show HLA-A.B.C (FIG. 9C) or PD-L1 (FIG. 9D) expression on the cell surface in A549 cells treated with the indicated drugs. Data are representative of four independent experiments (100 nM DAC, 5 μM GSK, and/or 200 nM CFI) (DMSO (A); CFI (B); DAC+GSK (C); DAC+GSK+CFI (D)). Mean fluorescence intensity (MFI) was quantified by FlowJo (right). FIG. 9E provides a schematic of an immune cell migration assay utilizing a 3D microfluidic device with tumor spheroids embedded in a central collagen-filled channel and with immune cells cocultured in a side channel. FIGS. 9F-9K provide representative images of Jurkat-CXCR3 (FIG. 9F, 9G) or NK-92 (FIG. 91, 9J) cell migration. Immune cell infiltration into the peri-tumor region is quantified by ImageJ (FIGS. 9H and 9K). Values were normalized to DMSO control. FIGS. 9L and 9M show an IB of the indicated proteins in patient-derived KL or KP cells (FIG. 9L), and DFCI-316 or DFCI-332 cells treated with 100 nM DAC, 5 μM GSK126, and/or 100 nM BAY-1217389 in accordance with pretreatment schedule as shown in FIG. 8B (FIG. 9M). FIG. 9N is a schematic of co-culture PBMC-derived T-cells with patient-derived KL cells pretreated with 100 nM DAC, 5 μM GSK126, and/or 100 nM BAY-1217389. FIG. 9O shows ELISA results of human granzyme B in CM derived from DFCI-316 cells co-cultured with PBMC-derived T-cells. FIGS. 9P-9Q show ELISA results of human CXCL10 in CM derived from DFCI-316 or DFCI-332 cells treated with 100 nM DAC, 5 μM GSK126, and/or 100 nM BAY-1217389 (FIG. 9P), and the ratio of infiltration of PBMC-derived T-cells into peri-tumor region utilizing immune cell migration assay (FIG. 9Q). p-values were calculated by unpaired two-tailed Student's t test (FIGS. 9F-9K), or one-way ANOVA followed by Tukey's post-hoc test (FIGS. 9C, 9D, 9O, 9Q) or two-way ANOVA followed by Sidak's post-hoc test (FIGS. 9A, 9B, 9P), *p<0.05, **p<0.01.

FIGS. 10A-10F provide data showing that MPS1 inhibition upregulates human leukocyte antigens (HLAs) expression and immune infiltration into the peri-tumor region. FIG. 10A shows HLA-A.B.C expression on the cell surface in H1944 or H647 cells transduced with the indicated vectors and treated with 100 nM BAY-1217389 (DMSO (A); BAY-1217389 (B)). FIG. 10B shows quantification by FlowJo of mean fluorescence intensity (MFI) of HLA-A.B.C or PD-L1 expression on the cell surface in A549 cells treated with the indicated drugs (100 nM DAC, 5 μM GSK, and/or 200 nM CFI) (see FIGS. 9C and 9D) (DMSO (A), left; DAC (B), center; GSK (C), right). FIGS. 10C and 10D show CXCR3 expression on the cell surface in Jurkat-CXCR3 cells (FIG. 10C) or NK-92 cells (FIG. 10D) (IgG (A); CXCR3 (B)). FIGS. 10E and 10F are representative images of Jurkat-CXCR3 (FIG. 10E) or NK-92 (10F) cell migration. Immune cells infiltration into the peri-tumor region is quantified by imageJ (bottom). Values were normalized to DMSO control. p-values were calculated by one-way ANOVA followed by Tukey's post-hoc test FIG. 10B, 10E, 10F), *p<0.05, **p<0.01.

FIGS. 11A-11L show that sequential combination therapy with MPS1 and DNMT inhibitor enhances intratumoral T cell infiltration in a syngeneic murine KL model. FIG. 11A shows an IB of the indicated proteins in murine lung cancer cells transduced with the indicated vectors. FIG. 11B shows qRT-PCR results of Sting in murine lung cancer cells treated with 100 nM DAC for 5 days (DMSO (A), left; DAC (B), right). FIG. 11C shows a heat map of cytokine profiles in CM derived from 393P-K or 393P-KL cells. Scores=log₂ fold change (393P-KL/393P-K). Cytokines indicating log₂ fold change (L2FC)>0.2 or L2FC<−0.2 are shown in the heat map. KL shading from top down, and K shading form bottom up. FIG. 11D show an IB of the indicated proteins, and FIG. 11E shows ELISA results of mouse CXCL10 levels in CM derived from 393P-KL cells treated with the indicated drugs (100 nM DAC, and/or 200 nM CFI or 100 nM BAY) in accordance with the pretreatment schedule (FIG. 11E—DMSO (A), left; CFI (B), center; and BAY-1217389 (C), right). FIG. 11F provides a schematic of a pharmacodynamics study with MPS1 and DNMT inhibitors in a syngeneic murine KL model. FIG. 11G shows an IB of the indicated proteins, and FIG. 11H shows qRT-PCR results of Cxcl10, in tumor tissues derived from mice treated with the indicated drugs (each group, n=4). FIGS. 11I-11L provide representative CD3 (FIGS. 11I and 11K) or CD8 (FIGS. 11J and 11L) IHC images and quantitative analysis from 393P-KL tumors treated with vehicle or with a combination of decitabine and BAY-1217389. Arrows highlight peri-tumoral localization (black) and intra-tumoral localization (gray) of CD3+ or CD8+ T cells. QuPath was used to quantify CD3+ or CD8+ T-cell infiltration. Scale bar, 200 PM. p-values were calculated by unpaired two-tailed Student's t test (FIGS. 11B, 11H, 11K, and 11L) or two-way ANOVA followed by Sidak's post-hoc test (FIG. 11E), *p<0.05, **p<0.01.

FIGS. 12A-12H provide data showing that sequential combination therapy with MPS1 and DNMT inhibitors enhances intratumoral T cell infiltration in a syngeneic murine KL model. FIG. 12A is an IB of the indicated proteins in GEMM-derived cell lines or 393P-KL cells. FIG. 12B shows qRT-PCR results of CXCL10 in 393P-KL cells treated with the indicated drugs (100 nM DAC, and/or 200 nM CFI or 100 nM BAY) in accordance with the pretreatment schedule (see FIG. 8B) (DMSO (A), left; CFI (B), center; BAY-1217389 (C), right). FIG. 12C shows the fold change (DAC treated/DMSO treated) of Cxcl10 expression in 393P-K or 393P-KL cells. The cells were treated with 100 nM DAC for 5 days. FIG. 12D shows the tumor volume of 393P-K or 393P-KL cells after subcutaneous inoculation into syngeneic 129S2/SvPasCrl mice followed by treatment of anti-PD1 antibody on day 7, 9, and 14 (as shown by arrows). FIG. 12E shows flow cytometric analysis of CD11b+Ly-6G+ cells in the TME derived from 393P-K or 393P-KL cells (n=6 in each group). *p=0.0568, unpaired two-tailed Student's t test. FIG. 12F provides representative images of hematoxylin and eosin (H&E) staining from 393P-KL tumors treated with vehicle, or decitabine and BAY-1217389. Scale bar, 200 μM. FIGS. 12G and 12H show quantitative analysis from 393P-KL tumors treated with vehicle or combination of decitabine and BAY-1217389 (FIG. 12G, Intratumoral; FIG. 12H, Peritumoral). p-values were calculated by unpaired two-tailed Student's t test (FIGS. 12C, 12G, 12H), or two-way ANOVA followed by Sidak's post-hoc test (FIG. 12B), *p<0.05, **p<0.01.

FIGS. 13A-13N demonstrate that sequential combination therapy shows durable therapeutic effect in a syngeneic murine KL model. FIG. 13A provides a schematic of short-term efficacy study, CD8+ T cell depletion study, and immune profiling with MPS1 and DNMT inhibitor in syngeneic murine KL model (Horizontal bar; decitabine treatment. BAY-1217389 treatment (second and third arrow from right)). FIG. 13B shows tumor volume of 393P-KL cells after subcutaneous inoculation into syngeneic 129S2/SvPasCrl mice treated with anti-CD8 neutralization antibody, DAC, and/or BAY-1217389 in accordance with the schedule as shown in FIG. 13J. FIG. 13C shows the mean tumor volume of 393P-KL cells after subcutaneous inoculation into syngeneic 129S2/SvPasCrl mice treated with anti-CD8 neutralization antibody. Mice were treated with anti-CD8 antibody, and/or DAC and BAY-1217389 in accordance with the schedule shown in FIG. 13A. Horizontal bar; decitabine treatment. Arrows; BAY-1217389 treatment. FIG. 13D shows the mean tumor volume of STING KO 393P-KL cells after subcutaneous inoculation into syngeneic 129S2/SvPasCrl mice. Mice were treated with DAC from day 1 to day 7 and BAY-1217389 on day 8 and 9. Horizontal bar; decitabine treatment. Arrows; BAY-1217389 treatment. FIG. 13E shows the flow cytometric analysis of immune cell populations in tumor tissue treated with or without decitabine and BAY-1217389 (n=5). Tumor tissue were collected and analyzed after 48 hours from second BAY-1217389 treatment. n.s., not significant. FIGS. 13F and 13L provide schematics of long-term efficacy studies with MPS1 inhibitor, DNMT inhibitor, and/or anti-PD1 antibody in syngeneic murine KL model. FIGS. 13G and 13I show tumor volume of 393P-KL cells (FIG. 13G) and mouse body weight (FIG. 13I) after subcutaneous inoculation into syngeneic 129S2/SvPasCrl mice followed by BAY-1217389 on day 8, 9, 21, and 22 (as shown with arrows) and/or decitabine from day 1 to day 7 (as shown by the bar). FIG. 13J provides a schematic of a CD8+ T cell depletion study with MPS1 and DNMT inhibitor in a syngeneic murine KL model (Horizontal bar; decitabine treatment. BAY-1217389 treatment (second and third arrow from right)). FIGS. 13H and K show tumor volume of 393P-KL cells (FIG. 13H) and mouse body weight (FIG. 13K) after subcutaneous inoculation into syngeneic 129S2/SvPasCrl mice followed by BAY-1217389 on day 8, 9, 21, and 22 (as shown with arrows) and/or decitabine from day 1 to day 7 (as shown by the bar). FIGS. 13M and 13N show the tumor volume of 393P-KL cells (FIG. 13M) and mouse body weight (FIG. 13N) after subcutaneous inoculation into syngeneic 129S2/SvPasCrl mice followed by BAY-1217389 on days 8 and 9 (as shown with arrows) decitabine from day 1 to day 7 (as shown by the bar), and/or anti-PD1 antibody on days 1, 4, 7, and 10 (as shown with arrows under the graph). p-values were calculated by two-way ANOVA followed by Sidak's post-hoc test (FIGS. 13C, 13D), unpaired two-tailed Student's t test (FIG. 13E), or χ² test (FIGS. 13H, 13M), *p<0.05, **p<0.01.p-values were calculated by χ² test (FIG. 13G), or one-way ANOVA followed by Tukey's post-hoc test (FIG. 13B), **p<0.01.

FIGS. 14A-14G provide data demonstrating that sequential combination therapy shows durable therapeutic effect in a syngeneic murine KL model. FIG. 14A shows a flow cytometric analysis of CD8+ and CD4+ T cells in the spleen (left) or tumor tissue (right) following the treatment of anti-CD8 neutralization antibody (n=2 in each group). Tumor tissues were analyzed at day 11. FIG. 14B shows the percentage change in tumor volume of 393P-KL cells inoculated into syngeneic 129S2/SvPasCrl mice day 9 after treatment with anti-CD8 neutralization antibody. n.s., not significant. p-values were calculated by unpaired two-tailed Student's t test (FIG. 14B). FIG. 14C shows the mean tumor volume of 393P-KL cells inoculated into syngeneic 129S2/SvPasCrl mice followed by treatment with anti-CD8 neutralization antibody on days 0, 1, 3, 6, 9, and 12. FIG. 14D shows the mean tumor volume of 393P-KL cells after subcutaneous inoculation into immunodeficient NSG mice. Mice were treated with DAC from day1 to day7 and BAY-1217389 on days 8 and 9. Bar; decitabine treatment. Arrows; BAY-1217389 treatment. FIG. 14E is an IB of the indicated proteins in 393P-KL cells transduced with the indicated vectors and treated with 100 nM DAC for 5 days. FIG. 14F shows the ELISA result of mouse CXCL10 levels in CM (FIG. 14D) derived from 393P-KL cells transduced with the indicated vectors and treated with 100 nM BAY-1217389 (DMSO (A), left; BAY-1217389 (B), right). FIG. 14G shows the flow cytometric analysis of immune cell populations in tumor tissue treated with or without decitabine and BAY-1217389 (n=5). Tumor tissue was collected and analyzed after 48 hours from second BAY-1217389 treatment. n.s., not significant. p-values were calculated by unpaired two-tailed Student's t test (FIG. 14G), or two-way ANOVA followed by Sidak's post-hoc test (FIG. 14F), *p<0.05, **p<0.01.

### DETAILED DESCRIPTION OF THE INVENTION

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the terms defined herein have the meanings ascribed to them unless specified otherwise.

The present disclosure is based on the unexpected discovery that treatment of KRAS-LKB1 (KL) mutant cancer cells with an epigenetic inhibitor, followed by pulse treatment with a DNA-damaging agent, restores STING signaling in STING-absent cancer cells and results in T cell infiltration and durable response in vivo, without evidence of significant toxicity. This sequential therapeutic approach utilizing inhibition of an epigenetic regulatory enzyme, followed by administration of a DNA-damaging agent such as an MPS1 inhibitor, reverses STING silencing and results in the production of micronuclei. Unexpectedly, the strategy described herein primarily impacts dividing cells and therefore preferentially targets quickly dividing cancer cells.

Thus, the present disclosure provides, inter alia, methods for treating a subject having cancer comprising administering to the subject a therapeutically effective amount of an epigenetic inhibitor, followed by a therapeutically effective amount of a DNA damaging agent. Also provided herein are methods for treating a subject having cancer based on the expression levels of STING and/or an epigenetic regulatory enzyme in a biological sample taken from the subject. Further provided herein are kits comprising an epigenetic inhibitor and a DNA damaging agent.

### Epigenetic Inhibitors

The present disclosure provides methods of treating a subject having cancer, comprising, in part, administering to the subject a therapeutically effective amount of an epigenetic inhibitor. “Epigenetic inhibitors” include any agents (including, for example, small molecules, nucleic acids, oligonucleotides, polypeptides, or proteins) that are capable of inhibiting an epigenetic modification to a nucleic acid. Epigenetic modifications to nucleic acids may include, for example, DNA methylation or demethylation, histone methylation or demethylation, and histone acetylation or deacetylation.

As used herein the term “inhibit” or “inhibition” in the context of enzymes, for example, in the context of an epigenetic regulatory enzyme (e.g., DMNT1), refers to a reduction in the activity of the enzyme. In some embodiments, the term refers to a reduction of the level of enzyme activity, e.g., the activity of an epigenetic regulatory enzyme, to a level that is statistically significantly lower than an initial level, which may, for example, be a baseline level of enzyme activity. In some embodiments, the term refers to a reduction of the level of enzyme activity, e.g., the activity of an epigenetic regulatory enzyme, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of enzyme activity.

In some embodiments, an epigenetic modification to an oligonucleotide is mediated by an epigenetic regulatory enzyme. Epigenetic regulatory enzymes include, but are not limited to, DNA methyltransferases (including, for example, DMNT1), histone methyltransferases (including, for example, EZH2), and histone deacetylases (including, for example, HDAC). Thus, in some embodiments, the epigenetic inhibitors used in the present disclosure are DMNT1 inhibitors, EZH2 inhibitors, or histone deacetylase inhibitors.

Exemplary DNA methyltransferase inhibitors include, e.g., azacitidine, decitabine, zebularine, NPEOC-DAC, CP-4200, RX-3117, cytosine analogues, thio-cytidine derivatives, e.g., T-dCyd and 5-aza-T-dCyd, decitabine-p-deoxyguanosine (SGI-110), SAM analogues, SAH analogues, SGI-1027, alcyne derivatives, cyclopenta derivatives, cyclohexathiophene derivatives, tryptophane derivates, e.g., RG108, procainamide derivatives, flavonoid derivatives, curcumin, psammaplin, hydralazine, disulfiram, 5-fluro-2′-deoxycitidine, 5-azacytidine, 5-aza-2′-deoxycytidine, 5,6-dihydro-5-azacytidine, 5-fluoro-2′-deoxycytidine, 1-(beta-D-ribofuranosyl)-1,2-dihydropyrimidin-2-one, 5-aza-2′-deoxycytidine-p-deoxyguanosine, fluorocyclopentenylcytosine, 2-(p-nitrophenyl) ethoxycarbonyl-5-aza-2′-deoxycytidine, 4′-thio-2′-deoxycytidine, 5-aza-4′-thio-2′-deoxycytidine, 1-beta-D-arabinofuranosyl-5-azacytosine, hydralazine, procaine, mithramycin A, nanaomycin A, N-(4-((2-amino-6-methylpyrimidin-4-yl)amino)phenyl)-4-(quinolin-4-ylamino)benzamide, N-phthalyl-L-tryptophan, N-phthalyl-L tryptophan derivatives, alkine derivatives, halomon, S-adenosyl-L-methionine analogues, S-adenosyl-L-homocysteine, S-adenosyl-L-homocysteine analogues, MG98, miR-29a, miR-29c, Sinefungin, procainamide, procainamide derivatives, procainamide-N-phthalyl-L-tryptophan conjugates, cyclopentathiophene derivatives, cyclohexathiophene derivatives, flavone derivatives, 3-nitroflavone derivatives, flavanones derivatives, 3-chloro-3-nitroflavanone derivatives, diclone, laccaic acid, acridine, 5,5′-Methylenedisalicylic acid, 4-(2-((5-Chloro-2-methoxybenzoyl)amino)ethyl)hydrocinnamic acid, 4-Chloro-N-(4-hydroxy-1-naphthalenyl)-3-nitro-benzenesulfonamide, (S)-3-(1H-Indol-3-yl)-2-(5-nitro-1,3-dioxo-1,3-dihydro-isoindol-2-yl)-propionic acid, (R)-2-(1,3-Dioxo-5-phenylethynyl-1,3-dihydro-isoindol-2-yl)-3-(1H-indol-3-yl)-propionic acid, (S)-2-(2,6-Dioxo-piperidin-1-yl)-3-(1H-indol-3-yl)-propionic acid, N-hydroxy-4-(2-(4-aminobenzamido)-ethylcarbamoyl)butanamide, 4-amino-N-(2-(ethyl(3-(hydroxyamino)-3-oxopropyl)amino)ethyl)benzamide, N<1>-(2-(4-aminobenzamido)ethyl)-N<1>-ethyl-N<6>-hydroxyadipamide, tetraethylthiuramdisulfide, (−)-epigallocatechin-3-gallate, genistein, psammaplin A, psammaplin derivatives, and anti-DNA methyltransferase antibodies. In some embodiments, the DNA methyltransferase inhibitor is a DMNT1 inhibitor. In certain embodiments, the DMNT1 inhibitor is decitabine.

Non-limiting examples of EZH2 inhibitors include S-adenosyl-methionine-competitive small molecule inhibitors. In particular non-limiting embodiments, the EZH2 inhibitor is derived from tetramethylpiperidinyl compounds. Further non-limiting examples include UNC1999, 3-Deazaneplanocin A (DZNcp), Ell, EPZ-5676, EPZ-6438, GSK343, EPZ005687, EPZ011989, GSK126, CAS #1346574-57-9, (S)-1-(sec-butyl)-N-((4,6-dimethyl-2-oxo-1,2-dihydropyridin-3-yl)methyl)-3-methyl-6-(6-(piperazin-1-yl)pyridin-3-yl)-1H-indole-4-carboxamide, DZNep, GSK126 tazemetostat, anti-EZH2 antibodies, and siRNA directed against EZH2.

Non-limiting examples of HDAC inhibitors include hydroxamic acids (e.g., trichostatin A, vorinostat (SAHA), belinostat (PXD101), LAQ824, Panobinostat (LBH589)), cyclic tetrapeptides (e.g., trapoxin B), depsipeptides, benzamides (e.g., entinostat (MS-275), tacedinaline (CI994), and mocetinostat (MGCD0103)), electrophilic ketones, aliphatic acid compounds (e.g., phenylbutyrate and valproic acid), nicotinamide, nicotinamide derivatives (e.g., dihydrocoumarin, napthopyranone, and 2-hydroxynathaldehydes), anti-HDAC antibodies, and siRNA directed against HDAC.

In some embodiments, administration of an epigenetic inhibitor to a subject in the methods disclosed herein results in increased activity of Stimulator of interferon genes (STING). In some embodiments, administration of an epigenetic inhibitor results in increased expression of STING. In some embodiments, administration of an epigenetic inhibitor results in restored expression of STING. STING is a ubiquitously produced transmembrane protein encoded by the TMEM173 gene. The longest isoform of STING has 379 amino acids. STING plays a key role as a mediator of innate immune signaling. It induces the innate immune signaling in response to the detection of bacterial and viral DNA in the cytoplasm and promotes the production of type I interferon (IFN-alpha and IFN-beta). Multiple studies have involved STING in the development of conditions including infectious diseases and certain cancers.

The STING amino acid sequence is:

(SEQ ID NO: 1)
MPHSSLHPSIPCPRGHGAQKAALVLLSACLVTLWGLGEPPEHTLRYLVLH
LASLQLGLLLNGVCSLAEELRHIHSRYRGSYWRTVRACLGCPLRRGALLL
LSIYFYYSLPNAVGPPFTWMLALLGLSQALNILLGLKGLAPAEISAVCEK
GNFNVAHGLAWSYYIGYLRLILPELQARIRTYNQHYNNLLRGAVSQRLYI
LLPLDCGVPDNLSMADPNIRFLDKLPQQTGDHAGIKDRVYSNSIYELLEN
GQRAGTCVLEYATPLQTLFAMSQYSQAGFSREDRLEQAKLFCRTLEDILA
DAPESQNNCRLIAYQEPADDSSFSLSQEVLRHLRQEEKEEVTVGSLKTSA
VPSTSTMSQEPELLISGMEKPLPLRTDFS

The STING amino acid sequence has GenBank Accession Number NP_938023.1. The STING nucleotide sequence has GenBank Accession Number NM_198282.3.

In some embodiments, a cancer, e.g., a tumor or cancer cell, with downregulated levels of STING expression and/or activity is susceptible to a combination therapy comprising administration of (i) an epigenetic inhibitor; and (ii) a DNA damaging agent. As used herein, a downregulated or reduced level includes a level that is below a control level or reference value as defined herein. A downregulated or reduced level may be, for example, 1%, 5%, 10%, 20%, 30%, 40%, 50%, 60%, 70%, 80%, 90%, 100%, 150%, 200%, 300%, 400%, 500% or more below a control level or reference value as defined herein. In some embodiments, STING levels are unchanged relative to controls.

As used herein, “STING activity” refers to the activation of STING signaling pathways. Without being bound by theory or mechanism, the activation of the STING signaling pathway stimulates TBK1 activity to phosphorylate IRF3 or Signal transducer and activator of transcription 6 (STAT6). Phosphorylated IRF3s and STAT6s dimerize and then enter the nucleus where they stimulate interferon related genes (e.g., Interferon Beta 1 (IFNB), C-C Motif Chemokine Ligand 2 (CCL2), C-C Motif Chemokine Ligand 20 (CCL20), C-X-C Motif Chemokine Ligand 10 (CXCL10), and C-C Motif Chemokine Ligand 5 (CCL5)).

### DNA Damaging Agents

The present disclosure provides methods of treating a subject having cancer, comprising, in part, administering to the subject a therapeutically effective amount of a DNA damaging agent. In some embodiments, the DNA damaging agent is administered to the subject following administration of an epigenetic inhibitor, as discussed herein. “DNA damaging agents” (including, for example, any small molecules, oligonucleotides, or proteins that cause DNA damage) are commonly used in cancer chemotherapy. In some embodiments, a DNA damaging agent disrupts mitosis. In certain embodiments, a DNA damaging agent results in mitotic catastrophe (e.g., through disruption of the mitotic spindle during cell division when a cell is treated with, for example, a taxane). In certain embodiments, a DNA damaging agent does not result in mitotic catastrophe, and cells are able to continue the next cycle of cell division following treatment with the DNA damaging agent. In some embodiments, such a DNA damaging agent induces formation of micronuclei in a cell.

Exemplary DNA damaging agents include, e.g., AZD0156, AZD1775, AZD6738, barasertib, BAY-1217389, bendamustine, bleomycin, ceralasertib, cisplatin, carboplatin, capecitabine, CC-671, CFI-402257, cyclophosphamide, doxorubicin, daunorubicin, docetaxel, etoposide, epirubicin, irinotecan, gemcitabine, ifosfamide, olaparib, oxaliplatin, LY2603618, melphalan, methotrexate, MK1775, MK5108, MK8776, MSC2490484A, niraparib, paclitaxel, pemetrexed, prexasertib, rucaparib, talazoparib, topotecan, vinorelbine, veliparib, volasertib, VX-970, and VX-984.

In some embodiments, a DNA damaging agent is a PARP inhibitor, an Aurora B inhibitor, an Aurora A inhibitor, a WEE1 inhibitor, an ATR inhibitor, a CHK1 inhibitor, an MPS1 inhibitor, or a PLK1 inhibitor. In some embodiments, a DNA damaging agent is an anti-folate drug. In certain embodiments, a DNA damaging agent is an inhibitor of monopolar spindle 1 (MPS1) kinase.

MPS1 inhibitors include, but are not limited to, BAY-1217389, BOS-172722, empesertib, AZ3146, CFI-402257, MPI-0479605, Mps1-IN-1, NMS-P715, Mps1-IN-3, Mps1-IN-2, CCT251455, TC-Mps1-12, anti-MPS1 antibodies, and MPS1-targeting siRNAs. In certain embodiments, the MPS1 inhibitor is BAY-1217389. In some embodiments, MPS1 is inhibited to a level that is, e.g., less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of MPS1 activity.

### Detection Methods

The methods and devices of the invention may be protein or mRNA based. Examples of protein-based assays include immunoassays (also referred to herein as immune-based assays), immunohistochemistry, flow cytometry, mass spectrometry, Western blots, Western immunoblotting, multiplex bead-based assays, and assays involving aptamers (such as SOMAmer™ technology) and related affinity agents. Examples of mRNA-based assays include Northern analysis, quantitative RT-PCR, microarray hybridization, and multiplex bead-based assays. These assays generally and commonly detect and measure the level of the biomarker of interest. The level of the biomarker may then be compared to a control level. Control levels will be discussed in greater detail herein.

mRNA Detection

The art is familiar with various methods for analyzing mRNA levels. An exemplary quantitative RT-PCR assay may be carried out as follows: mRNA is extracted from cells in a biological sample (e.g., tumor cells) using the RNeasy kit (Qiagen). Total mRNA is used for subsequent reverse transcription using the SuperScript III First-Strand Synthesis SuperMix (Invitrogen) or the SuperScript VILO cDNA synthesis kit (Invitrogen). The RT reaction is used for quantitative PCR using SYBR Green PCR Master Mix and gene-specific primers, in triplicate, using an ABI 7300 Real Time PCR System.

Expression profiles of cells in a biological sample (e.g., tumor cells) can be carried out using an oligonucleotide microarray analysis. It is to be understood that such arrays may however also comprise positive and/or negative control markers such as housekeeping genes that can be used to determine if the array has been degraded and/or if the sample has been contaminated. The art is familiar with the construction of oligonucleotide arrays. See for example GeneChip Human Genome U133 Plus 2.0 Affymetrix expression array (Affymetrix). Other mRNA detection methods include multiplex detection assays well known in the art, e.g., xMAP® bead capture and detection (Luminex Corp., Austin, TX), and various oligonucleotide array assays (Illumina).

mRNA Detection Binding Partners

mRNA detection binding partners include oligonucleotide or modified oligonucleotide (e.g. locked nucleic acid) probes that hybridize to a target mRNA. Methods for designing and producing oligonucleotide probes are well known in the art (see, e.g., U.S. Pat. No. 8,036,835; Rimour et al. GoArrays: highly dynamic and efficient microarray probe design. Bioinformatics (2005) 21 (7): 1094-1103; and Wernersson et al. Probe selection for DNA microarrays using OligoWiz. Nat Protoc. 2007; 2(11):2677-91).

### Protein Detection

The art is familiar with various methods for analyzing protein levels. An exemplary immunoassay may be carried out as follows: A biological sample is applied to a substrate having bound to its surface biomarker-specific binding partners (i.e., immobilized biomarker-specific binding partners). The biomarker-specific binding partner (which may be referred to as a “capture ligand” because it functions to capture and immobilize the biomarker on the substrate) may be antibodies or antigen-binding antibody fragments such as Fab, F(ab)2, Fv, single chain antibodies, Fab and sFab fragments, F(ab′)2, Fd fragments, scFv, and dAb fragments, although they are not so limited. Other binding partners are described herein. Biomarkers present in the biological sample bind to the capture ligands, and the substrate is washed to remove unbound material. The substrate is then exposed to soluble biomarker-specific binding partners (which may be identical to the binding partners used to immobilize the biomarker). The soluble biomarker-specific binding partners are allowed to bind to their respective biomarkers immobilized on the substrate, and then unbound material is washed away. The substrate is then exposed to a detectable binding partner of the soluble biomarker-specific binding partner. In one embodiment, the soluble biomarker-specific binding partner is an antibody having some or all of its Fc domain. Its detectable binding partner may be an anti-Fc domain antibody. As will be appreciated by those in the art, if more than one biomarker is being detected, the assay may be configured so that the soluble biomarker-specific binding partners are all antibodies of the same isotype. In this way, a single detectable binding partner, such as an antibody specific for the common isotype, may be used to bind to all of the soluble biomarker-specific binding partners bound to the substrate.

It is to be understood that the substrate may comprise capture ligands for one or more biomarkers, including two or more, three or more, four or more, five or more, etc. of the biomarkers provided by the invention.

In some instances, it may be preferable to measure biomarkers having the lowest detectable concentration. An example would be biomarkers having protein concentrations in the μg/ml range. In some instances, it may be preferable to measure, on a single substrate, biomarkers having protein concentrations that are in the same dynamic range (i.e., they are present in the biological sample in the same concentration range). Those of ordinary skill in the art will be able to devise multiplexing assays (i.e., assays that measure two or more markers) using the guidance provided herein and the knowledge in the art.

In some embodiments, the invention contemplates a substrate having a pre-determined amount of capture ligands for each biomarker. The pre-determined amount of capture ligand may be based in part on prior measurements of biomarker levels in subjects that are STING high and STING low. The pre-determined amount of capture ligand may be based in part on prior measurements of biomarker levels in subjects that are high for one or more SPARCS genes and low for one or more SPARCS genes. The assays may be designed such that if the subject is STING or SPARCS-positive, then one or more detectable signals appear, optionally on a biomarker-by-biomarker basis.

Other examples of protein detection methods include multiplexed immunoassays as described, e.g., in U.S. Pat. Nos. 6,939,720 and 8,148,171, and published US Patent Application No. 2008/0255766, and protein microarrays as described, e.g. in published US Patent Application No. 2009/0088329.

### Protein Detection Binding Partners

Protein detection binding partners include biomarker-specific binding partners. In some embodiments, binding partners may be antibodies. As used herein, the term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab′)2, Fd fragments, Fv fragments, scFv, and dAb fragments) as well as complete antibodies. Methods for making antibodies and antigen-binding fragments are well known in the art (see, e.g. Sambrook et al, “Molecular Cloning: A Laboratory Manual” (2nd Ed.), Cold Spring Harbor Laboratory Press (1989); Lewin, “Genes IV”, Oxford University Press, New York, (1990), and Roitt et al., “Immunology” (2nd Ed.), Gower Medical Publishing, London, New York (1989), WO2006/040153, WO2006/122786, and WO2003/002609).

Binding partners also include proteins or peptides that bind to or interact with a target biomarker, e.g. through non-covalent bonding. For example, if the biomarker is a ligand, a binding partner may be a receptor for that ligand. In another example, if the biomarker is a receptor, a binding partner may be a ligand for that receptor. In yet another example, a binding partner may be a protein or peptide known to interact with a biomarker. Methods for producing proteins are well known in the art (see, e.g. Sambrook et al, “Molecular Cloning: A Laboratory Manual” (2nd Ed.), Cold Spring Harbor Laboratory Press (1989) and Lewin, “Genes IV”, Oxford University Press, New York, (1990)) and can be used to produce binding partners such as ligands or receptors.

Binding partners also include aptamers and other related affinity agents. Aptamers include oligonucleic acid or peptide molecules that bind to a specific target molecule. Methods for producing aptamers to a target molecule are well known in the art (see, e.g., published US Patent Application No. 2009/0075834, U.S. Pat. Nos. 7,435,542, 7,807,351, and 7,239,742). Other examples of affinity agents include SOMAmer™ (Slow Off-rate Modified Aptamer, SomaLogic, Boulder, CO) modified nucleic acid-based protein binding reagents.

Binding partners also include any molecule capable of demonstrating selective binding to any one of the protein targets disclosed herein, e.g., peptoids (see, e.g., Reyna J Simon et al., “Peptoids: a modular approach to drug discovery” Proceedings of the National Academy of Sciences USA, (1992), 89(20), 9367-9371; U.S. Pat. No. 5,811,387; and M. Muralidhar Reddy et al., Identification of candidate IgG biomarkers for Alzheimer's disease via combinatorial library screening. Cell 144, 132-142, Jan. 7, 2011).

### Controls

In some embodiments, methods provided herein involve measuring a level of a biomarker in a biological sample and comparing the biomarker level to a control level. The control level is a level of the same biomarker in a control tissue, control subject, or a population of control subjects. The “control” may be (or may be derived from) a normal subject (or normal subjects). Normal subjects, as used herein, refer to subjects that are apparently healthy and show no cancer symptoms. The control population may therefore be a population of normal subjects. In some embodiments, the control is from a normal healthy subject or subjects and is from the same tissue type as the biological sample.

### Treatment of Cancer

The present disclosure provides methods of treating a subject having cancer. The present disclosure also provides kits which may be useful in the treatment of cancer.

Cancers include, but are not limited to: Oral: buccal cavity, lip, tongue, mouth, pharynx; Cardiac: sarcoma (angiosarcoma, fibrosarcoma, rhabdomyosarcoma, liposarcoma), myxoma, rhabdomyoma, fibroma, lipoma and teratoma; Lung: non-small cell lung cancer (NSCLC), small cell lung cancer, bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Karposi's sarcoma, leiomyoma, hemangioma, lipoma, neurofibroma, fibroma), large bowel or large intestines (adenocarcinoma, tubular adenoma, villous adenoma, hamartoma, leiomyoma), rectal, colon, colon-rectum, colorectal; Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma, biliary passages; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), head and neck cancer, meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, SertolI-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma] hairy cell; lymphoid disorders; Skin: malignant melanoma, basal cell carcinoma, squamous cell carcinoma, Karposi's sarcoma, keratoacanthoma, moles dysplastic nevi, lipoma, angioma, dermatofibroma, keloids, psoriasis, Thyroid gland: papillary thyroid carcinoma, follicular thyroid carcinoma; medullary thyroid carcinoma, multiple endocrine neoplasia type 2A, multiple endocrine neoplasia type 2B, familial medullary thyroid cancer, pheochromocytoma, paraganglioma; and Adrenal glands: neuroblastoma.

In some embodiments, the subject has lung cancer. In some embodiments, the subject has non-small cell lung cancer. In certain embodiments, the cancer is a KRAS-LKB1 (KL) mutant cancer as described herein. The data presented herein demonstrate that KL mutant lung cancer may be effectively treated by administering an epigenetic inhibitor, followed by a DNA damaging agent. As such, in some embodiments, a subject having KL mutant lung cancer is treated with (i) a therapeutically effective amount of an epigenetic inhibitor; and (ii) a therapeutically effective amount of a DNA damaging agent.

Also contemplated in the present disclosure are methods for treating cells in vitro, comprising administering an epigenetic inhibitor and a DNA damaging agent to one or more cells or tissue. The cells can be cancerous or non-cancerous. In some embodiments, the cells are treated with an epigenetic inhibitor and a DNA damaging agent to determine response to the treatment or effectiveness of the treatment.

### KRAS-LKB1 Mutant Lung Cancer

Non-small-cell lung cancer (NSCLC) is a heterogeneous disease, with multiple different oncogenic mutations. Approximately 25-30% of NSCLC patients present KRAS mutations, which confer poor prognosis and high risk of tumor recurrence. In the majority of cases, these KRAS mutations are missense mutations which introduce an amino acid substitution at position 12, 13, or 61 (e.g., an amino acid substitution at position 12, 13, or 61 of NCBI NP_004976.2). The result of these mutations is constitutive activation of KRAS signaling pathways. The role of KRAS mutations and their potential association with other common genetic lung cancer lesions (LKB1, P53) has recently been investigated in different cohorts of human lung adenocarcinomas using transcriptional, mutational, copy-number, and proteomic data. These studies highlighted that LKB1 inactivation is significantly associated with KRAS mutations compared to P53 deletion, and that co-occurrence of KRAS mutation with inactivation of LKB1 or P53 genes generates different tumor subsets with distinct biology, immune profiles, and therapeutic vulnerabilities. About half of NSCLCs with activating KRAS lesions also have deletions or inactivating mutations in the serine/threonine kinase 11 (LKB1) gene. Loss of LKB1 on a KRAS-mutant background may represent a significant source of heterogeneity, contributing to poor response to therapy. LKB1 mutations associated with lung cancer are extensively characterized in the art. Exemplary LKB1 mutations can be found, for example, in Kaufman et al., Cancer Research, 2016, the entirety of which is incorporated herein by reference.

The genotype of lung cancer can be determined by means readily known to those of skill in the art for assessing the genotype and/or levels of the markers, e.g., KRAS, LKB1, and p53, as described, for example, in Sholl, (Transl Lung Cancer Res. 2017 6(5): 560-569) including but not limited to, determining the genomic sequence of a marker, e.g., by DNA sequencing or allele specific PCR, determining expression of the marker, e.g., by northern analysis, quantitative PCR, or microarray analysis, or determining protein levels of the marker, e.g., by western analysis, mass spectrometry, immunohistochemistry, etc.

The terms “administer,” “administering,” or “administration,” as used herein refer to implanting, absorbing, ingesting, injecting, inhaling, or otherwise introducing the one or more therapeutic agents (e.g., an epigenetic inhibitor and/or a DNA damaging agent).

As used herein, the term “treating” refers to the application or administration of a composition including one or more active agents to a subject, who has cancer, a symptom of cancer, or a predisposition toward the disease, with the purpose to cure, heal, alleviate, relieve, alter, remedy, ameliorate, improve, or affect the disorder, the symptom of the disease, or the predisposition toward the disease. In some embodiments, the cancer is a KRAS-LKB1 mutant lung cancer.

Alleviating cancer includes delaying the development or progression of the disease or reducing disease severity. Alleviating the disease does not necessarily require curative results. As used therein, “delaying” the development of a disease (such as cancer) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of cancer includes initial onset and/or recurrence.

An “effective amount” or “therapeutically effective amount” refers to an amount sufficient to elicit the desired biological response, i.e., treating the cancer. As will be appreciated by those of ordinary skill in this art, the effective amount of the compounds described herein may vary depending on such factors as the desired biological endpoint, the pharmacokinetics of the compound, the condition being treated, the mode of administration, and the age and health of the subject. An effective amount includes, but is not limited to, that amount necessary to slow, reduce, inhibit, ameliorate, or reverse one or more symptoms associated with cancer. For example, in the treatment of cancer, such terms may refer to a reduction in the size of the tumor.

When administered to a subject, effective amounts of the therapeutic agent will depend, of course, on the particular disease being treated; the severity of the disease; individual patient parameters including age, physical condition, size and weight, concurrent treatment, frequency of treatment, and the mode of administration. These factors are well known to those of ordinary skill in the art and can be addressed with no more than routine experimentation. In some embodiments, a maximum dose is used, that is, the highest safe dose according to sound medical judgment.

In the treatment of a subject having cancer, an effective amount is that amount which slows the progression of the cancer (e.g., the growth of the tumor—as determined by size, metastasis), halts the progression of the disease, or reverses the progression of the disease. An effective amount includes that amount necessary to slow, reduce, inhibit, ameliorate or reverse one or more symptoms associated with the cancer. Disease progression can be monitored by clinical observations, laboratory and imaging investigations apparent to a person skilled in the art. A therapeutically effective amount can be an amount that is effective in a single dose or in a multi-dose therapy (e.g., an amount that is administered in two or more doses or administered chronically).

Chronic treatments include forms of repeated administration for an extended period of time (e.g., for one or more months, between a month and a year, one or more years, or longer). In many embodiments, a chronic treatment involves administering the compositions of the present disclosure repeatedly over the duration of illness of the patient. In general, a suitable dose such as a daily dose of a therapeutic agent or combination of therapeutic agents described herein will be that amount of the structure that is the lowest dose effective to produce a therapeutic effect. Such an effective amount will generally depend upon the factors described above.

The epigenetic inhibitors and DNA damaging agents provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a compound required to achieve an effective amount will vary from subject to subject, depending, for example, on species, age, and general condition of a subject, severity of the side effects or disorder, identity of the particular compound, mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

As used herein, the term “in combination” refers to the use of more than one therapeutic agent. The use of the term “in combination” does not restrict the order in which the therapeutic agents are administered to a subject. In some embodiments, a first therapeutic agent, such as an epigenetic inhibitor, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), the administration of a second therapeutic agent, such as a DNA damaging agent, to a subject.

“Subject” means a mammal, such as a human, a nonhuman primate, a dog, a cat, a sheep, a horse, a cow, a pig or a goat. In an important embodiment, the mammal is a human. The subject as used herein can be an adult subject or a pediatric subject. In some embodiments, the subject has or is suspected of having cancer, e.g., any of the cancers described herein. In some embodiments, the subject is at elevated risk of developing cancer, for example, due to the presence of carcinogenic genetic mutations or exposure to carcinogens or radiation.

In some embodiments, the subject to be treated by the methods described herein is human. In some embodiments, a human subject who needs the treatment may be a human patient having, at risk for having, or suspected of having cancer. A subject having cancer can be identified by routine medical examination, e.g., laboratory tests, functional tests, biopsy, CT scans, or ultrasounds. A subject suspected of having cancer might show one or more symptoms of the disorder. A subject at risk for cancer can be a subject having one or more of the risk factors for that disorder. For example, risk factors associated with cancer include (a) hereditary cancer, (b) age, and (c) family history of cancer.

A “biological sample” from a subject can include any cellular, tissue, bone marrow, or blood sample from the subject. Any type of biological sample appropriate for conducting assays described herein can be compatible with aspects of the invention, as would be understood by one of ordinary skill in the art. In some embodiments, the biological sample is tumor tissue or a biopsy sample.

### Timing of Administration

The steps of the methods disclosed herein can be performed in any order. In certain preferred embodiments, the step of administering the epigenetic inhibitor to the subject is performed prior to the step of administering the DNA damaging agent. Without being bound by theory, treatment of cancer cells in which STING expression has been reduced, or in which STING expression is completely absent, with an epigenetic inhibitor can re-write chromatin in the cancer cells and turn STING expression back on. The cancer cells may then be sensitized to treatment with a DNA damaging agent, which may be administered at some point following administration of the epigenetic inhibitor.

In some embodiments, a subject is treated with an epigenetic inhibitor over the course of multiple days to allow multiple rounds of cell division to occur, effectively resetting chromatin to allow for increased expression of STING. In some embodiments, the epigenetic inhibitor is administered to the subject for about 1, about 2, about 3, about 4, about 5, about 6, about 7, about 8, about 9, about 10, about 11, about 12, about 13, or about 14 days. In some embodiments, the epigenetic inhibitor may be administered to a subject for longer than 14 days. In certain embodiments, the epigenetic inhibitor is administered to the subject for 7 days.

In some embodiments, following administration of the epigenetic inhibitor, a DNA damaging agent is administered to the subject as described herein. The DNA damaging agent may be administered to the subject using a pulse treatment strategy (e.g., discontinuous, or intermittent, treatment with high doses of the DNA damaging agent). In some embodiments, a DNA damaging agent is administered to the subject over the course of multiple days. In some embodiments, the DNA damaging agent is administered (optionally using a pulse therapy strategy) to the subject for about 1, about 2, about 3, about 4, about 5, about 6, or about 7 days. In some embodiments, the DNA damaging agent is administered to the subject for more than 7 days. In certain embodiments, the DNA damaging agent is administered to the subject (optionally using a pulse therapy strategy) for 2 days.

In some embodiments, the methods described herein further comprise administering a therapeutically effective amount of a DNA damaging agent to the subject an additional time following the first administration. In some embodiments, the same DNA damaging agent is administered to the subject in additional time. In some embodiments, administering a DNA damaging agent an additional time comprises administering to the subject a therapeutically effective amount of a different DNA damaging agent. The step of administering a DNA damaging agent an additional time may be performed any amount of time after a DNA damaging agent is administered to the subject. In some embodiments, the DNA damaging agent is administered to the subject about 1 week, about 2, weeks, about 3 weeks, or about 4 or more weeks after the DNA damaging agent is administered a first time. In certain embodiments, the DNA damaging agent is administered an additional time about 2 weeks after the DNA damaging agent is administered to the subject a first time.

### Kits

Also encompassed by the disclosure are kits (e.g., pharmaceutical packs). The kits provided may comprise a pharmaceutical agent described herein (e.g., an epigenetic inhibitor and/or a DNA damaging agent) and a container (e.g., a vial, ampule, bottle, syringe, and/or dispenser package, or other suitable container). In some embodiments, provided kits may optionally further include a second container comprising a pharmaceutical excipient as described herein for dilution or suspension of the epigenetic inhibitor and/or the DNA damaging agent. In some embodiments, the epigenetic inhibitor and DNA damaging agent described herein provided in the first container and the pharmaceutical excipient in the second container are combined to form one unit dosage form. In some embodiments, the kits provide more than one epigenetic inhibitor and/or more than one DNA damaging agent. In some embodiments, the kits provide more than one pharmaceutically acceptable excipient.

Thus, in one aspect, provided are kits including a first container comprising an epigenetic inhibitor and a DNA damaging agent described herein. In certain embodiments, the kits are useful for treating a disease (e.g., cancer) in a subject in need thereof. In certain embodiments, the kits are useful for preventing a disease (e.g., cancer) in a subject in need thereof. In certain embodiments, the kits are useful for reducing the risk of developing a disease (e.g., cancer) in a subject in need thereof. In certain embodiments, the kits are useful for inhibiting the activity (e.g., aberrant activity, such as increased activity) of an epigenetic regulatory enzyme in a subject or cell. In certain embodiments, the kits are useful for increasing or restoring expression of STING or activation of the STING signaling pathway in a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., cancer) in a subject in need thereof. In certain embodiments, the kits and instructions provide for preventing a disease (e.g., cancer) in a subject in need thereof. In certain embodiments, the kits and instructions provide for reducing the risk of developing a disease (e.g., cancer) in a subject in need thereof. In certain embodiments, the kits and instructions provide for inhibiting the activity (e.g., aberrant activity, such as increased activity) of an epigenetic regulatory enzyme in a subject or cell. In certain embodiments, the kits and instructions are useful for increasing or restoring expression of STING or activation of the STING signaling pathway in a subject or cell. A kit described herein may also include one or more additional pharmaceutical agents described herein as a separate composition.

Relative amounts of the epigenetic inhibitor, the DNA damaging agent, the excipient, and/or any additional ingredients in a kit of the disclosure will vary, depending upon the identity, size, and/or condition of the subject treated. Additional pharmaceutically acceptable excipients may be used in the manufacture of the provided kits. These include inert diluents, dispersing and/or granulating agents, surface-active agents and/or emulsifiers, disintegrating agents, binding agents, preservatives, buffering agents, lubricating agents, and/or oils. Excipients such as cocoa butter and suppository waxes, coloring agents, and coating agents may also be present in the kit.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80)), polyoxyethylene esters (e.g., polyoxyethylene monostearate (MYRJ 45), polyoxyethylene hydrogenated castor oil, polyethoxylated castor oil, polyoxymethylene stearate, and Solutol), sucrose fatty acid esters, polyethylene glycol fatty acid esters (e.g., Cremophor™), polyoxyethylene ethers, (e.g., polyoxyethylene lauryl ether (BRIJ 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC F-68, Poloxamer-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS, PHENONIP, methylparaben, GERMALL 115, GERMABEN II, NEOLONE, KATHON, and EUXYL.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

In some embodiments, the epigenetic inhibitors and/or DNA damaging agents of the present disclosure comprise a pharmaceutically acceptable salt. The term “pharmaceutically acceptable salt” refers to those salts which are, within the scope of sound medical judgment, suitable for use in contact with the tissues of humans and lower animals without undue toxicity, irritation, allergic response, and the like and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, non-toxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N+(C₁-C₄ alkyl)4—salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Liquid dosage forms (e.g., for parenteral administration) include pharmaceutically acceptable emulsions, microemulsions, solutions, suspensions, syrups and elixirs. In addition to the active ingredients (e.g., epigenetic inhibitors and DNA damaging agents), the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Although the descriptions of the kits provided herein are principally directed to kits which comprise therapeutic agents (e.g., epigenetic inhibitors and DNA damaging agents) that are suitable for administration to humans, it will be understood by the skilled artisan that such agents in the kits provided herein are generally suitable for administration to animals of all sorts. Modification of kits comprising therapeutic agents suitable for administration to humans in order to render them suitable for administration to various animals is well understood, and the ordinarily skilled veterinary pharmacologist can design and/or perform such modification with ordinary experimentation.

The therapeutic agents (e.g., epigenetic inhibitors and DNA damaging agents) provided herein are typically formulated in a size (e.g., volume) and weight appropriate for the intended use for ease of administration. It will be understood, however, that the total amount of the therapeutic agents in the kits of the present disclosure will be decided by the attending clinician or physician within the scope of sound medical judgment. The specific therapeutically effective dose level for any particular subject will depend upon a variety of factors including the disease being treated and the severity of the disorder; the activity of the specific active ingredient employed; the specific epigenetic inhibitor and DNA damaging agent employed; the age, body weight, general health, sex, and diet of the subject; the time of administration, route of administration, and rate of excretion of the specific active ingredient employed; the duration of the treatment; the drugs used in combination or coincidental with the specific active ingredient employed; and like factors well known in the medical arts.

As described herein, the therapeutic agents in the kits of the present disclosure can also be administered in combination with one or more additional pharmaceutical agents. For example, the epigenetic inhibitor and DNA damaging agent can be administered in combination with additional pharmaceutical agents that reduce and/or modify their metabolism, inhibit their excretion, and/or modify their distribution within the body. It will also be appreciated that the additional therapy employed may achieve a desired effect for the same disorder, and/or it may achieve different effects.

### EXAMPLES

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### Example 1: MPS1 Inhibition Primes Immunogenicity of KRAS-LKB1 Mutant Lung Cancer

Immune checkpoint blockade (ICB) exhibits significant therapeutic efficacy in many cancers, including non-small cell lung cancer (NSCLC). Recent efforts have identified biological markers that are predictive of favorable response, including higher PD-L1 expression, the degree of tumor mutation burden (TMB), and the number of tumor-infiltrating lymphocytes (TILs) residing in the tumor microenvironment (TME) (Keenan et al., 2019). On the other hand, certain somatic mutations, including those that impair interferon sensing or MHC class I display, allow cancer cells to evade cancer immunosurveillance and promote resistance to ICB (Keenan et al., 2019; Spranger and Gajewski, 2018).

Aberrant cytoplasmic DNA accumulation following invasion of microbial pathogens or cytosolic leakage of self-DNA can be detected by cyclic GMP-AMP synthase (cGAS), which produces the second messenger 2′3′-cyclic GMP-AMP (2′3′-cGAMP) that directly activates its downstream target, stimulator of interferon genes (STING) (Li and Chen, 2018). STING then translocates from the endoplasmic reticulum (ER) toward the ER-Golgi intermediate compartment (ERGIC) and subsequently activates TANK-binding kinase 1 (TBK1) to phosphorylate and activate IRF3 (Hopfner and Hornung, 2020; Zhang et al., 2020). Since IRF3-induced cytokines, including type I interferon and CXCL10, play a central role in enhancing antigen presentation and cytotoxic T-cell recruitment into tumors, intact STING signaling has been recognized as a determinant of therapeutic antitumor immunity (Kwon and Bakhoum, 2020).

The physiological function of the STING pathway has been studied in immune cells such as antigen-presenting cells (APCs) (Deng et al., 2014; Woo et al., 2014). However, it has been thought that the activity of the cancer cell-intrinsic STING pathway defines the immunogenicity and efficacy of ICB (Falahat et al., 2021; Falahat et al., 2019; Guan et al., 2021; Lu et al., 2021; Mahadevan et al., 2021). STING expression has been shown to be epigenetically silenced in KRAS; LKB1 (KL) mutated NSCLC cells through EZH2-mediated histone H3K27 methylation as well as DNMT1-induced 5-methylcytosine accumulation around its promoter (Kitajima et al., 2019). KL cells exhibit cytoplasmic accumulation of mitochondrial DNA due to an autophagic defect, which could select for STING silencing to protect cells from STAT1-induced cytotoxicity. Loss of tumor cell STING signaling in KL cells has been shown to impair infiltration of cytotoxic T-cells into the TME (Campisi et al., 2020), and is associated with resistance to anti-PD-1 therapy in the clinic (Koyama et al., 2016; Rizvi et al., 2018; Skoulidis et al., 2018). Re-engagement of STING activity might represent a promising strategy to restore immunogenicity of KL tumors. It has been shown that STING over-expression in KRAS mutated NSCLC cells with lentiviral vectors induced STAT1-dependent cell death along with elevated secretion of STING downstream cytokines specifically in KL cells, in contrast to LKB1 wild-type (WT) cells (Kitajima et al., 2019).

Synthetic STING agonists such as ADU-S100 and MK-1454 have been developed based on the structure of natural STING ligand cyclic di-nucleotides (CDN) (Kwon and Bakhoum, 2020). However, stimulation of cancer cell-intrinsic STING signaling by these molecules is limited by low cell-membrane permeability due to the intrinsic negative charges and hydrophilicity, while immune cells express import channels such as SLC19A1 and SLC46A2 (Cordova et al., 2021; Luteijn et al., 2019; Ritchie et al., 2019). Furthermore, exogenous STING agonist injection can promote T cell cytotoxicity and death (Gulen et al., 2017).

An alternative strategy to activate cancer cell-intrinsic STING signaling is to promote endogenous 2′3′-cGAMP production via cGAS activation. Studies have shown that genotoxic therapies, including radiation therapy and treatment with targeted or cytotoxic DNA-damaging agents, can trigger activation of the STING pathway through aberrant accumulation of cytoplasmic DNA in a cGAS-dependent manner (Reislander et al., 2020). For example, treatment with poly ADP-ribose polymerase (PARP) inhibitors such as olaparib causes genomic instability leading to cytosolic leakage of self-DNA specifically in homologous recombination (HR) deficient cancer cells associated with BRCA1/2 mutation (Ding et al., 2018; Pantelidou et al., 2019; Reislander et al., 2019). In addition, treatment with certain chemotherapy reagents, such as cisplatin and paclitaxel, induces cancer cell-intrinsic STING signaling via accumulation of DNA damage (Grabosch et al., 2019; Zierhut et al., 2019). However, most KL cells are BRCA1/2-proficient, and there is no a priori evidence that this or any other previously described approach is optimal to re-engage STING signaling in KL cells.

Although cGAS expression is often suppressed in certain types of cancer cells such as melanoma (Konno et al., 2018), most KL cell lines still express cGAS (Kitajima et al., 2019). Forcing KL cells to accumulate cytoplasmic DNA could be an effective approach to impair their viability and to increase their immunogenicity. The unique sensitivity of KL cells to intracellular accumulation of 2′3′-cGamp was examined and a screen was performed to identify clinical stage drugs that could co-opt this vulnerability, especially in combination with epigenetic therapies that force them to express STING.

### Results

LKB1 Inactivation Sensitizes KRAS Mutant NSCLC Cells to 2′3′-cGAMP

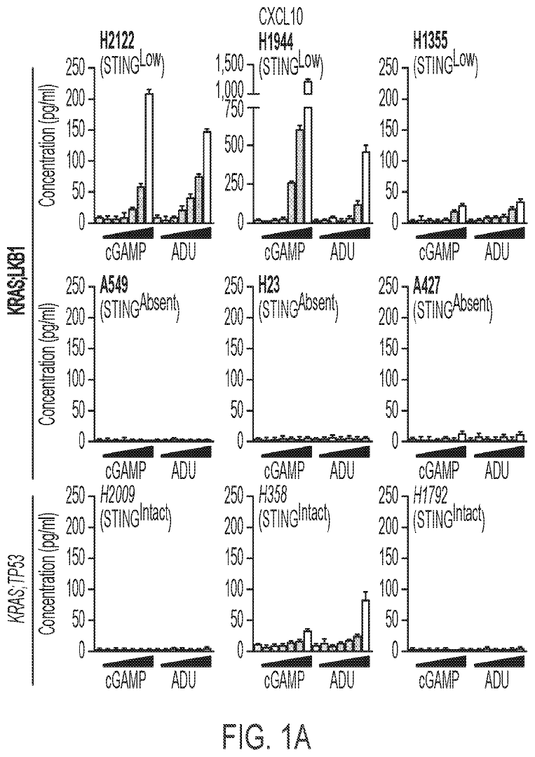

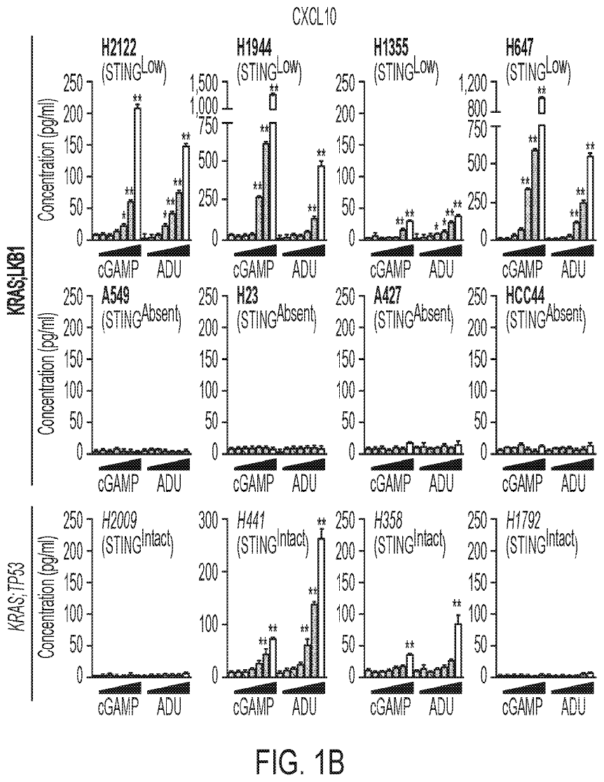

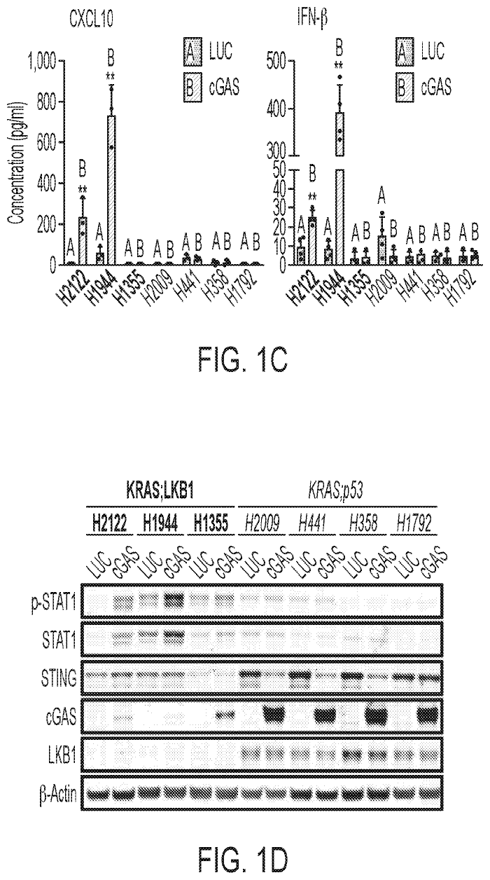

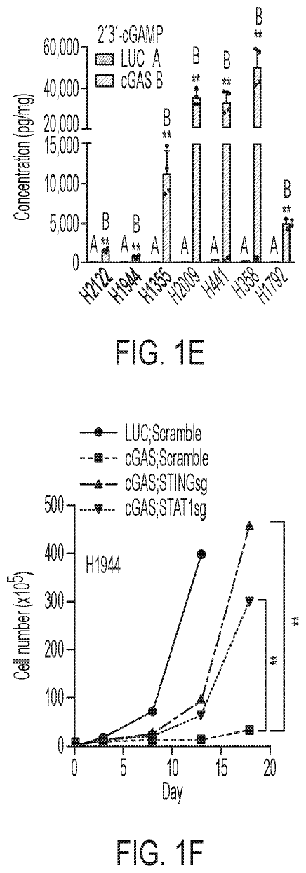

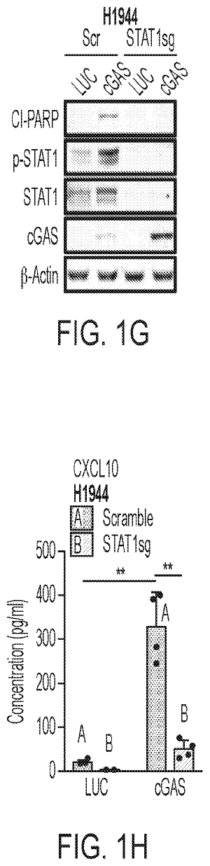

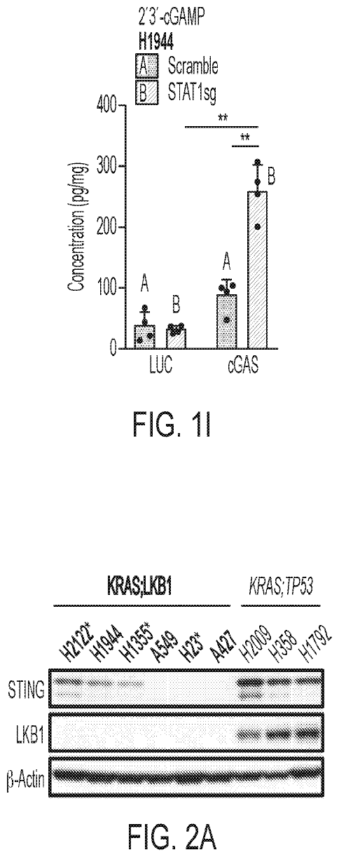

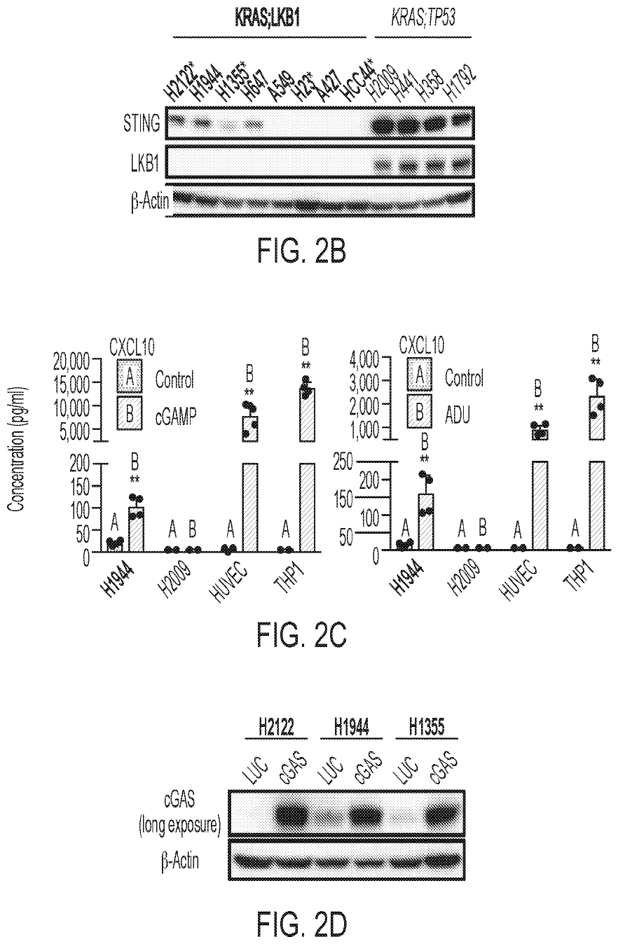

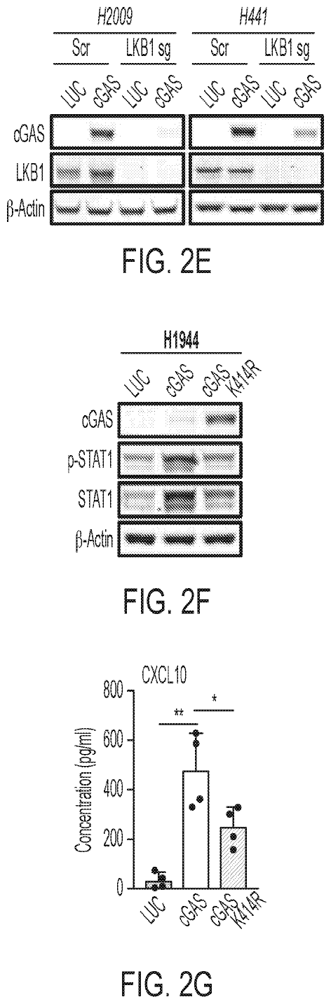

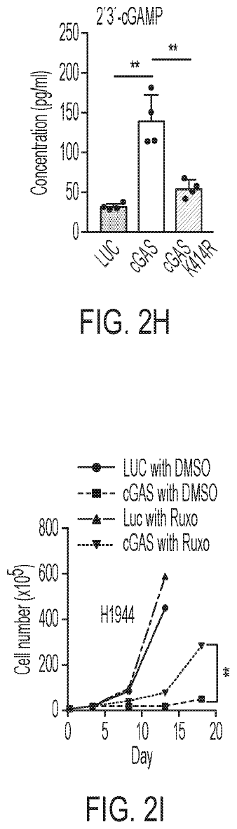

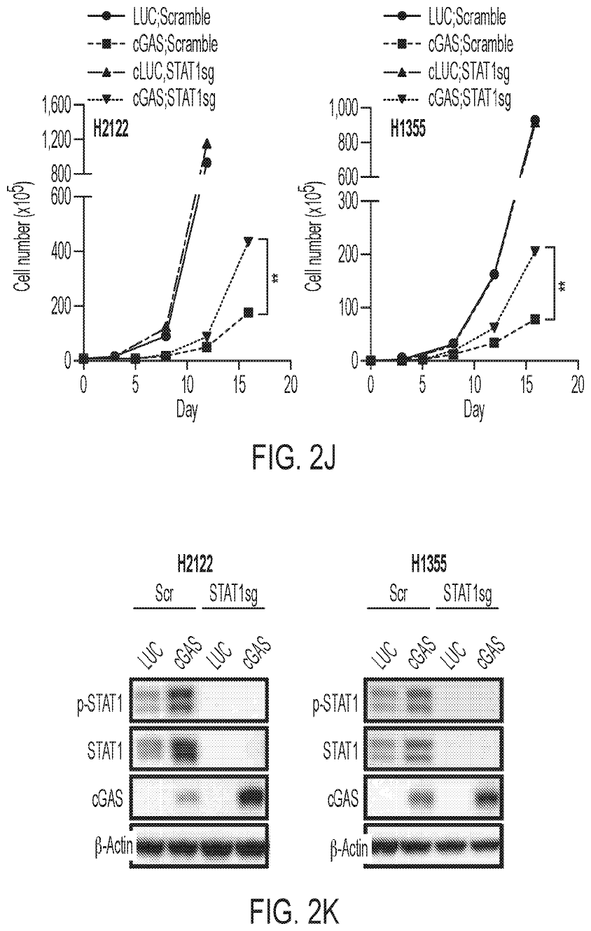

Treatment with 2′3′-cGAMP or ADU-S100 was utilized to examine the sensitivity of KP or KL NSCLC cell lines to exogenous CDN exposure. A subset of KL cell lines maintains low levels of STING protein expression (H2122, H1944, and H1355 cells—STING^{Low}), whereas others exhibit undetectable STING levels due to concurrent high DNMT1 expression (A549, H23, and A427 cells—STING^{Absent}) (Kitajima et al., 2019). STING^{Low} KL cells secreted CXCL10 in response to either 2′3′-cGAMP or ADU-S100 in a dose-dependent manner, especially H1944, H2122, and H647 cells that retain higher STING levels compared with H1355 cells (see FIG. 2B); in contrast, STING^{Absent} cell lines failed to respond to exogenous CDNs even at high doses, consistent with their lack of STING expression (FIGS. 1A, 1B). Notably, KP cell lines (H2009, H358, H441, and H1792 cells) exhibited generally weaker response to STING agonists in spite of their higher STING expression, with 2/4 KP cell lines such as H2009 and H1792 lacking response even at high dose (FIGS. 1A, 1B, 2A, 2B). KL cell response to extracellular CDNs was still modest as compared with endothelial cells (HUVEC) or monocytic THP-1 cells, which also have different membrane permeabilities (FIG. 2C) (Cordova et al., 2021; Luteijn et al., 2019; Ritchie et al., 2019). However, these findings suggested that STING-positive KL cell lines might be sensitive to CDNs and intracellular accumulation of 2′3′-cGAMP.

Next, sensitivity of these cancer cell lines was compared to endogenous intracellular 2′3′-cGAMP by forced over-expression of cGAS across multiple STING^{Low} KL vs KP cancer cell lines. H2122 and H1944 cGAS-expressing cells produced significantly higher levels of both CXCL10 and IFN-β relative to a luciferase control and failed to downregulate STING levels as a counter-regulatory response, in contrast to KP cells (FIGS. 1C, D). These same KL cell lines also uniquely activated STAT1 following cGAS transduction, in contrast to KP cells, despite achieving much lower levels of cGAS over-expression (FIGS. 1D, 2D) and generating significantly lower levels of 2′3′-cGAMP as compared to KP cell lines (FIGS. 1D, 1E). These data suggested hypersensitivity of the KL cellular state to intracellular 2′3′-cGAMP. Indeed, H1944 KL cells could tolerate over-expression of a catalytically inactive mutant cGAS (K414R) (Dai et al., 2019); and, LKB1 depletion hindered wild type cGAS accumulation KP cells (FIGS. 2D-2H). Together, these data suggest that LKB1 inactivation is not only associated with STING silencing, but also cGAS intolerance and 2′3′-cGAMP hypersensitivity.

It was previously determined that STAT1 activation contributes to KL intolerance of STING signaling (Kitajima et al., 2019). Its role in mediating this upstream intolerance to cGAS expression and 2′3′-cGAMP generation was therefore also assessed. Indeed, inhibition of STAT1, as well as STING, similarly attenuated cGAS-induced growth suppression, induction of apoptosis, and CXCL10 production in multiple STING^{Low} KL cell lines including H1944, H2122, and H1355 cells (FIGS. 1F-H, 21, and 2J). Furthermore, STAT1-depleted STING^{Low} KL cells exhibited higher exogenous cGAS expression and intracellular 2′3′-cGAMP accumulation following cGAS over-expression (FIGS. 1G, 1I, and 2K). Thus, KL cells appear to limit intracellular 2′3′-cGAMP accumulation at least in part to avoid the downstream cytotoxicity associated with activation of STAT1.

Identification of MPS1 Inhibition as a Potent Inducer of Endogenous 2′3′-cGAMP Production in KL Cells

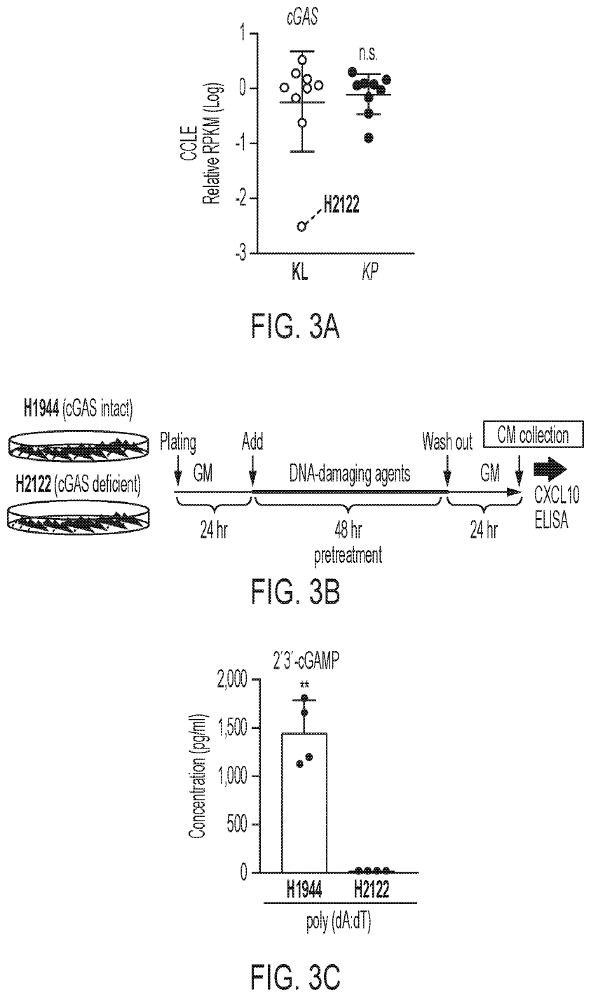

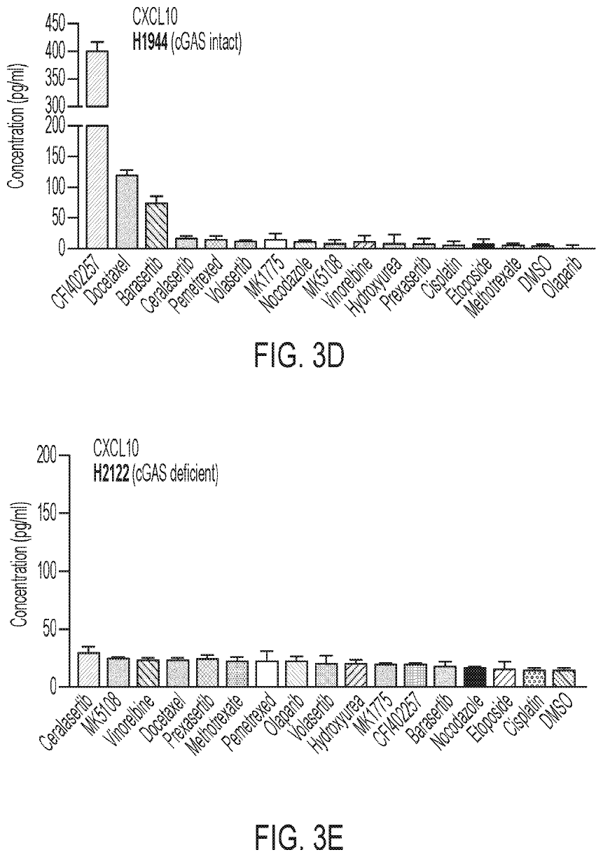

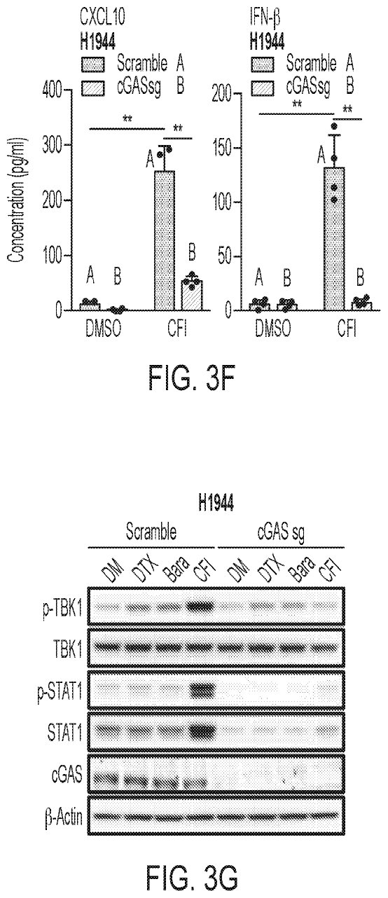

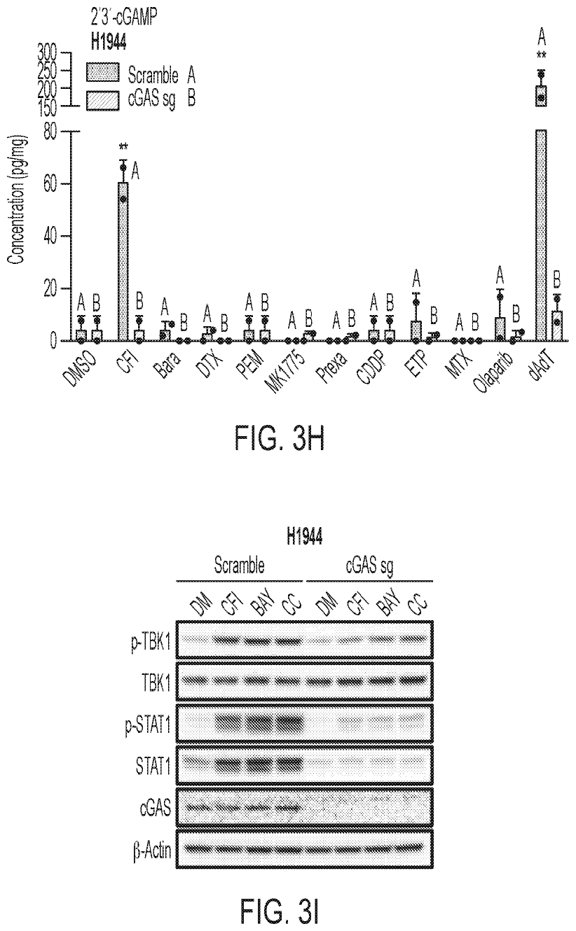

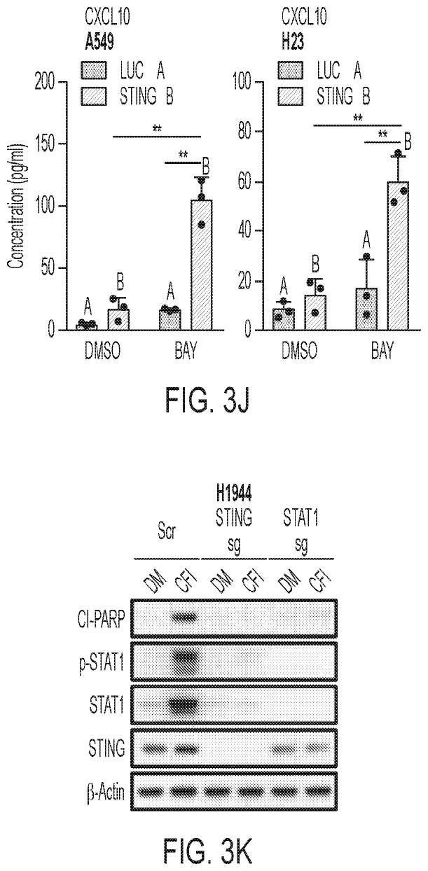

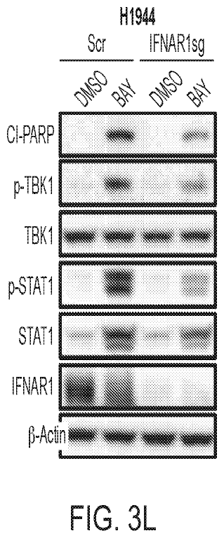

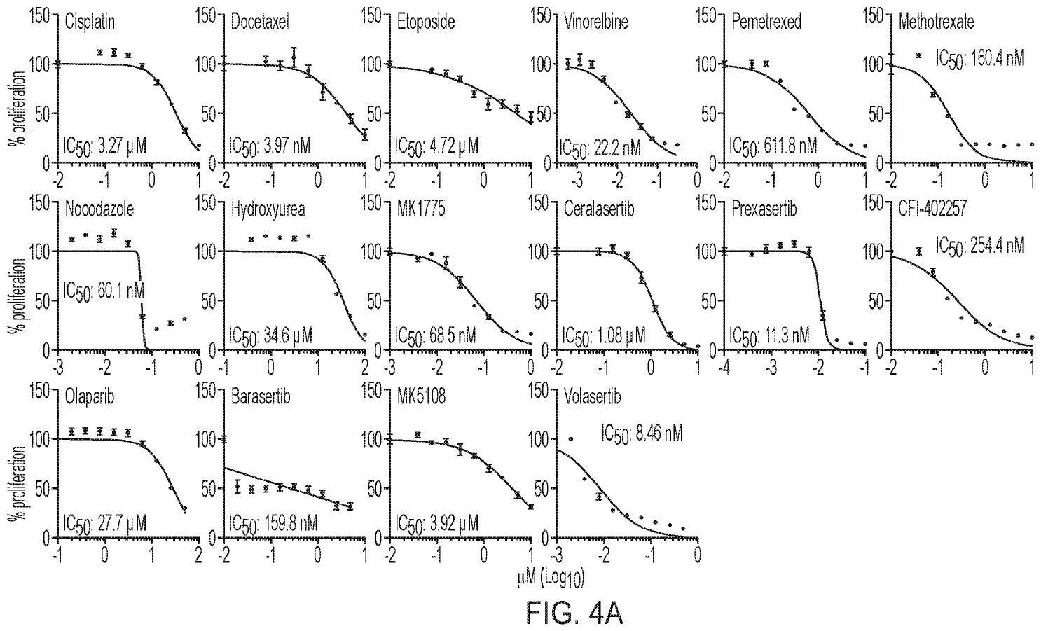

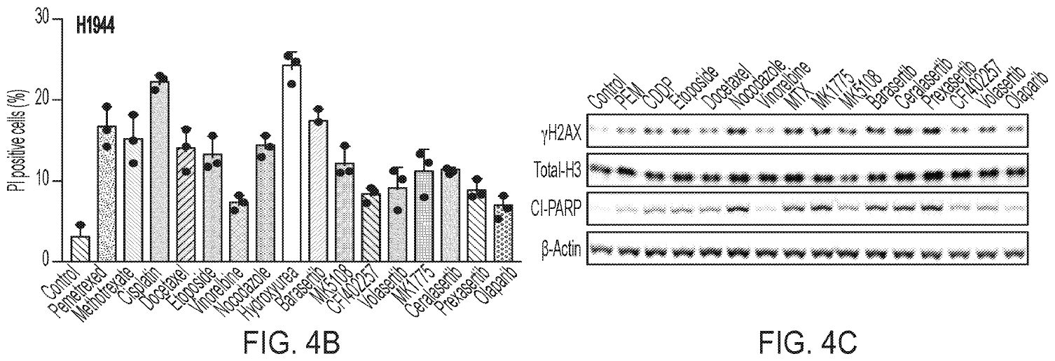

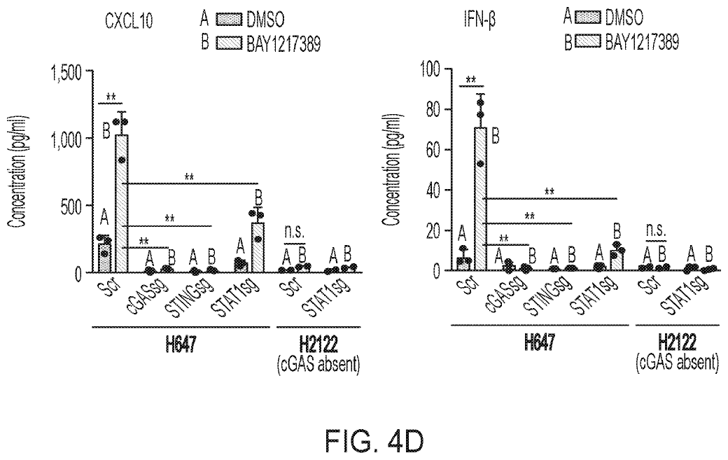

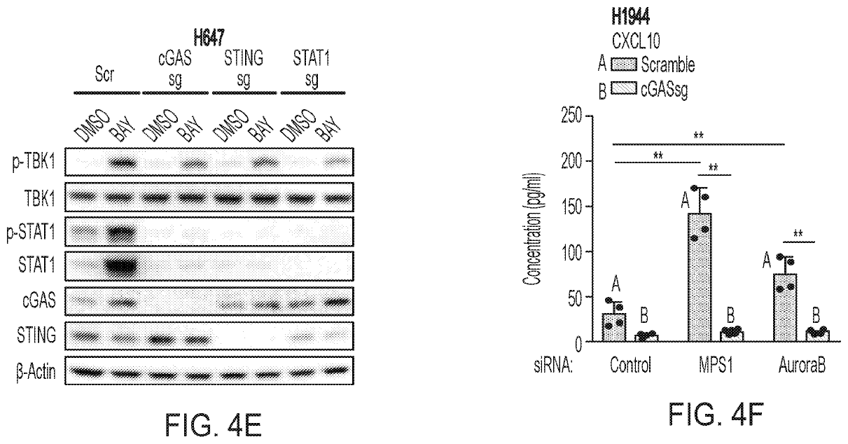

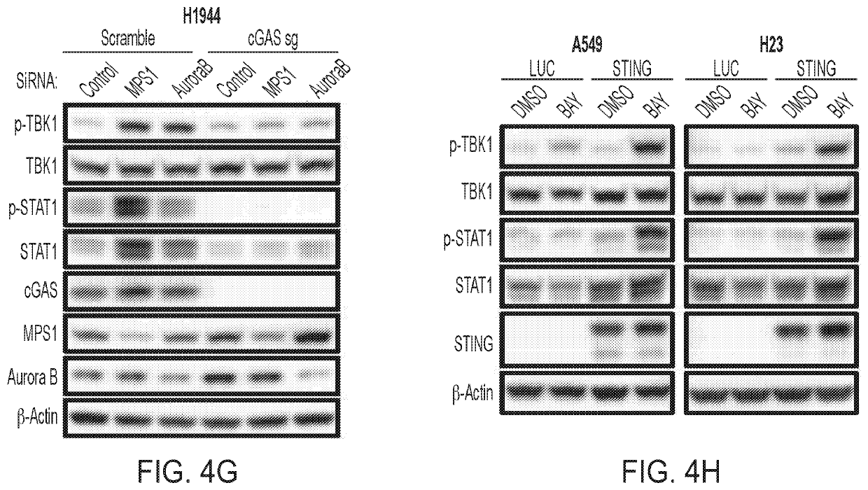

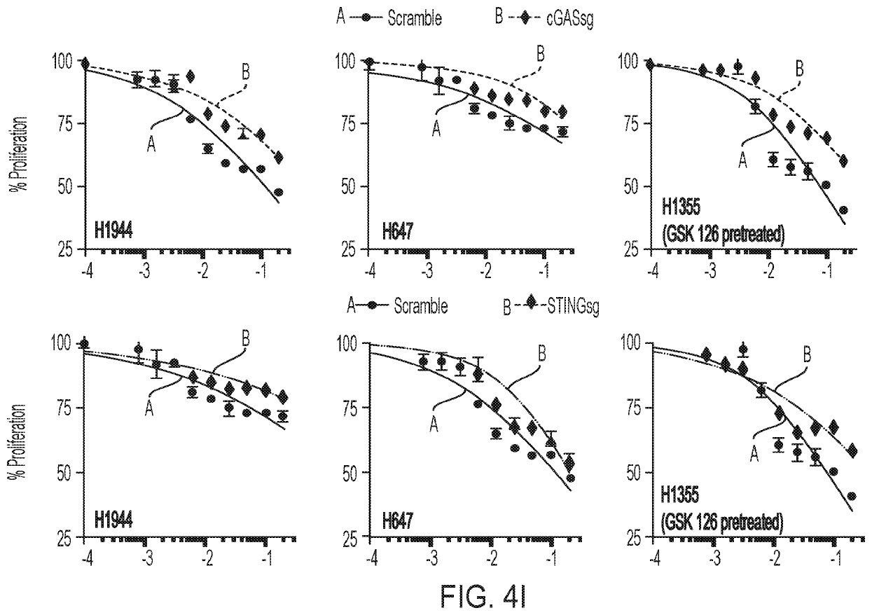

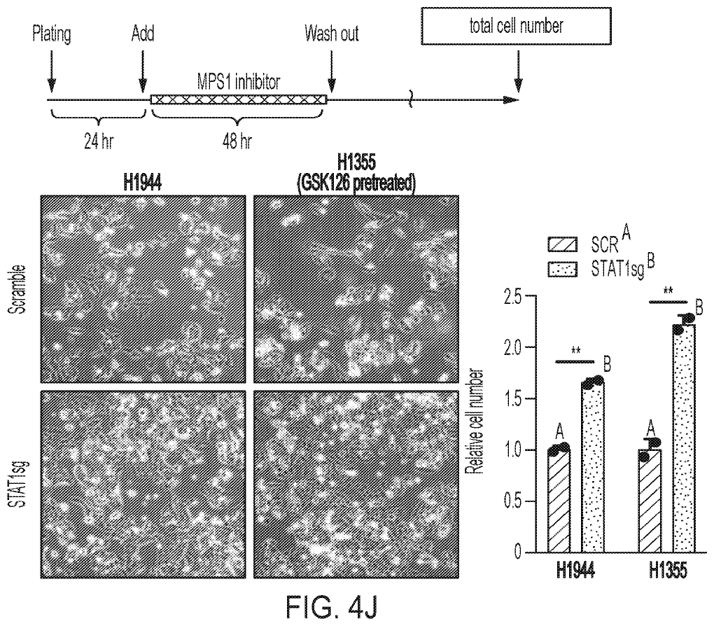

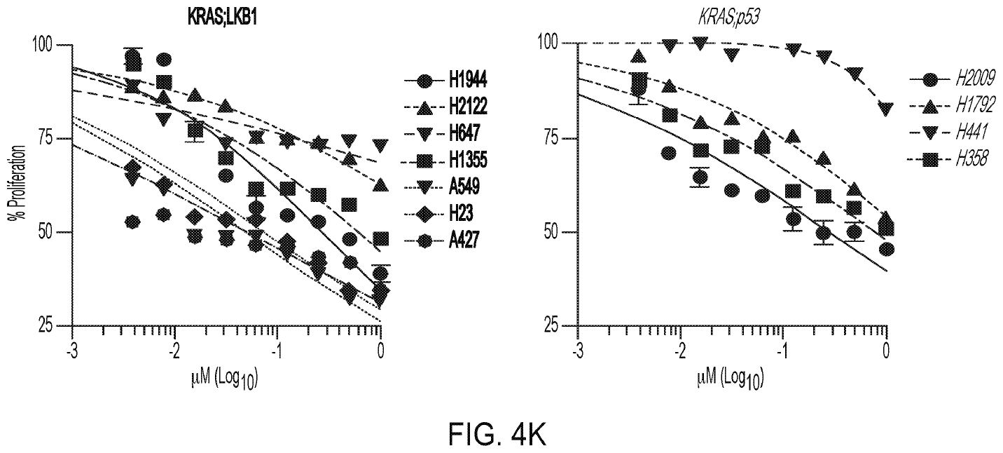

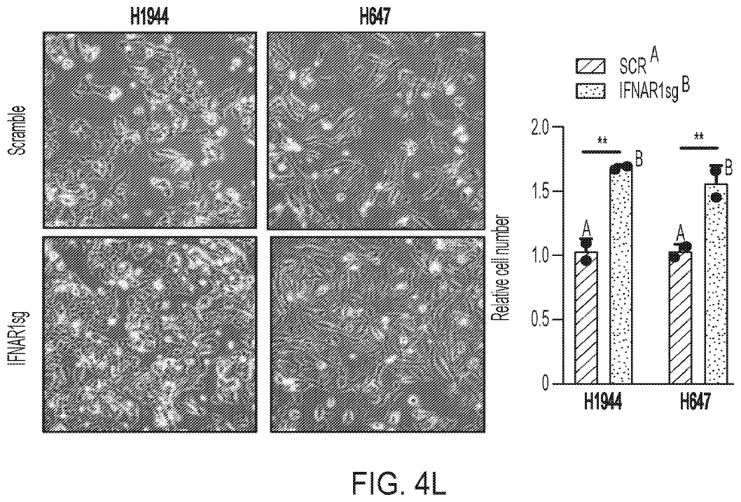

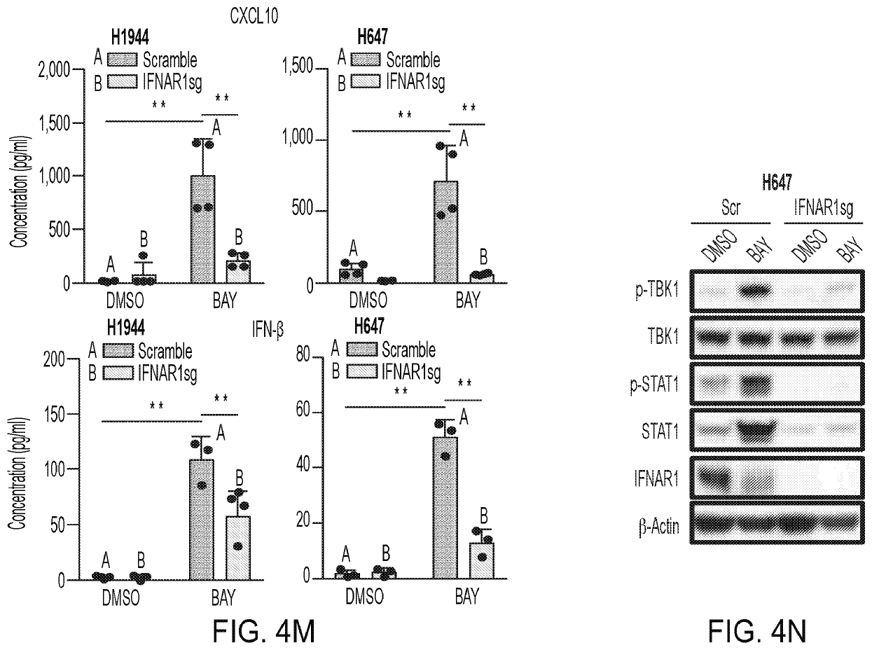

Due to their particular hypersensitivity, H1944 cells were utilized as a model system in which to conduct an unbiased screen of cytotoxic chemotherapies or targeted DNA-damaging agents for their ability to upregulate endogenous 2′3′-cGAMP production following pulse treatment, including cGAS-deficient H2122 cells as a counter-screen (FIGS. 3A and 3B). Indeed, selective induction of 2′3′-cGAMP by the dsDNA mimic poly(dA:dT) in H1944 cells, but not H2122 cells (FIG. 3C), was seen. DNA-damaging agents already utilized in the clinic or in clinical trials were tested; including a variety of chemotherapy drugs, including cisplatin, docetaxel, etoposide, vinorelbine, pemetrexed, and methotrexate, and molecularly targeted drugs against the DNA replication/repair pathways, including olaparib (PARP inhibitor), barasertib (Aurora B inhibitor), MK5108 (Aurora A inhibitor), MK1775 (WEE1 inhibitor), ceralasertib (ATR inhibitor), prexasertib (CHK1 inhibitor), CFI-402257 (MPS1 inhibitor), and volasertib (PLK1 inhibitor) were examined. Additionally, hydroxyurea or nocodazole were included as controls to induce S-phase or M-phase cell cycle arrest. The IC50 was identified for each compound for cell viability in H1944 cells, and this concentration was utilized for the screen (FIG. 4A and Table 1). It was shown that each agent induced cellular cytotoxicity and/or DNA-damage response at this concentration, as measured by the induction of propidium iodide positive cells, cleaved PARP fragments, or histone TH2A.X expression (FIGS. 4B and 4C).

TABLE 1
List of IC₅₀ in H1944 cells and the concentration
used for the screening of each DNA-damaging agent

| | | |
|---|---|---|
| Drug | IC50 | Conc. for the screening |

| | | | | |
|---|---|---|---|---|
| cisplatin | 3.27 | μM | 2.5 | μM |
| docetaxel | 3.97 | nM | 5 | nM |
| etoposide | 4.72 | μM | 5 | μM |
| vinorelbine | 22.2 | nM | 20 | nM |
| pemetrexed | 611.8 | nM | 500 | nM |
| methotrexate | 160.4 | nM | 200 | nM |
| nocodazole | 60.1 | nM | 50 | nM |
| hydroxyurea | 34.6 | μM | 50 | μM |
| olaparib | 27.7 | μM | 25 | μM |
| barasertib | 159.8 | nM | 200 | nM |
| MK5108 | 3.92 | μM | 5 | μM |
| MK1775 | 68.5 | nM | 50 | nM |
| ceralasertib | 1.08 | μM | 1 | μM |
| prexasertib | 11.3 | nM | 10 | nM |
| CFI-402257 | 254.4 | nM | 200 | nM |
| volasertib | 8.46 | nM | 5 | nM |

Three compounds were identified that could induce significant CXCL10 secretion in H1944 cells following 48-hour pulse treatment, all of which were anti-mitotics: docetaxel, barasertib, and especially CFI-402257, a selective inhibitor of the spindle assembly checkpoint (SAC) kinase monopolar kinase (MPS1; also known as TTK protein kinase) (Mason et al., 2017) (FIG. 3D). The degree of CXCL10 induction did not correlate with the amount of cytotoxicity or DNA-damage response induced by each agent, suggesting that the mechanism was not related to the degree of DNA-damage induced (FIGS. 4B and 4C). Furthermore, the ability of each agent to induce CXCL10 in H2122 cells was abrogated, suggestive of cGAS-dependence (FIG. 3E). Indeed, it was shown that CXCL10 and IFNβ secretion in H1944 cells following pulse treatment with CFI-402257, the most potent agent, was markedly attenuated by cGAS depletion (FIG. 3F), as was STAT1 activation (FIG. 3G). CFI-402257 was also the only agent that yielded detectable levels of intracellular 2′3′-cGAMP, which was similarly ablated by cGAS depletion (FIG. 3H).

To validate on-target effects of CFI-402257 treatment, two different MPS1 inhibitors (BAY-1217389 and CC-671) already utilized in clinical trials were examined, and it was shown that STING downstream is upregulated to the same extent following the treatment of each drug in a cGAS-dependent manner (FIG. 3I). It was also shown that MPS1 inhibitor-induced cellular cytotoxicity was dependent upon intact STING and STAT1 (FIG. 3K). Taken together, as compared to other DNA damaging agents that activate cGAS-STING in other contexts (Ding et al., 2018; Grabosch et al., 2019; Pantelidou et al., 2019; Reislander et al., 2019; Zierhut et al., 2019), these data revealed pulse MPS1 inhibition as the most potent inducer of intracellular 2′3′-cGAMP and downstream STING pathway activation in KL cells.

To rule out potential off-target effects of CFI-402257 treatment, the effects of two additional MPS1 inhibitors, BAY-1217389 and CC-671, which induced similar cGAS dependent activation of STING signaling in H1944 cells, were also examined (FIG. 3I); all of these findings were reproducible in an additional STING^{Low} KL cell line (FIGS. 4D and 4E). In addition, genetic depletion of MPS1 using siRNA significantly upregulated STING downstream signaling and CXCL10 secretion more potently than Aurora B depletion, in a cGAS dependent manner (FIGS. 4F and 4G). On the other hand, reconstitution of STING in STING^{absent} KL cells such as A549 and H23 cells also sensitized them to MPS1 inhibition, leading to CXCL10 secretion and STAT1 activation (FIGS. 3J, 4H). It was determined that MPS1 inhibitor-induced cellular cytotoxicity and growth arrest in KL cells required activation of STING signaling following 48-hour pulse treatment, and to a lesser degree during continuous 96 hour SAC inhibition, which is also capable of arresting KP cells (FIGS. 3K, 4I-4K). Notably, IFNAR1 depletion also attenuated cell death and growth arrest, and CXCL10 induction by pulse MPS1 inhibitor treatment in KL cells (FIGS. 4L and 4M). Phosphorylation of TBK1 and IFN-β secretion, which are upstream of IFNAR1, were also attenuated by IFNAR1 depletion (FIGS. 3L, 4M, and 4N). These data implied that additional formation of a positive feedback loop between STING signaling and type 1 IFN contributed to their hypersensitivity to pulse MPS1 inhibition, potentially independently of its direct anti-mitotic effects.

### MPS1 Inhibition Activates the STING Pathway Via Micronuclei Formation in KL Cells

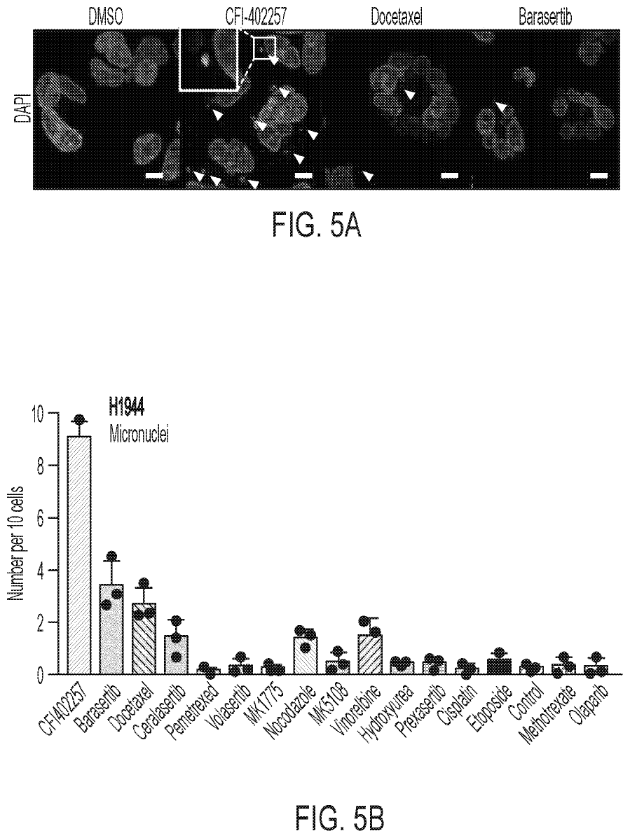

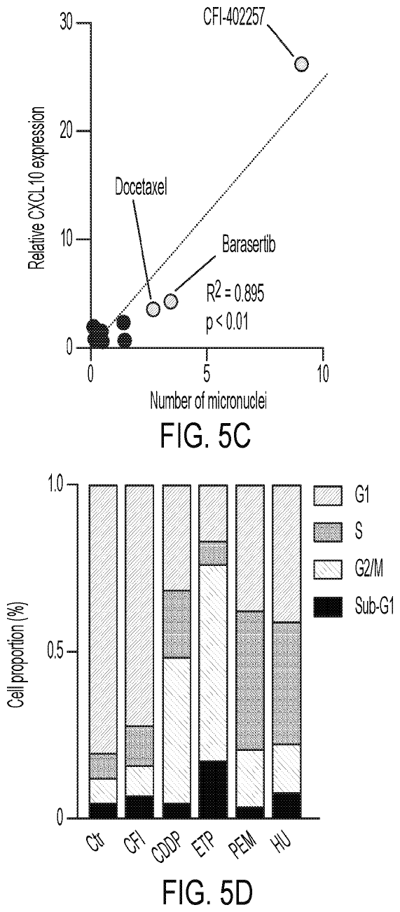

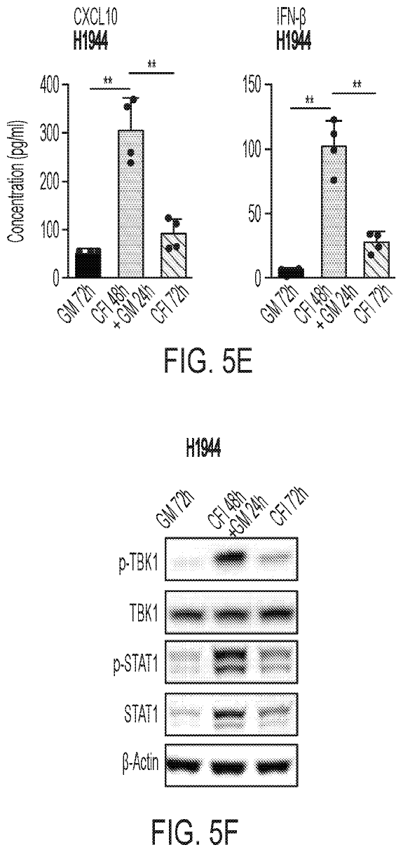

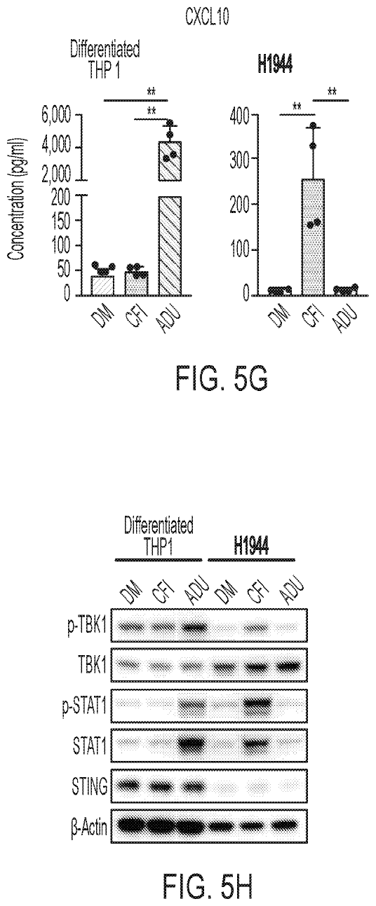

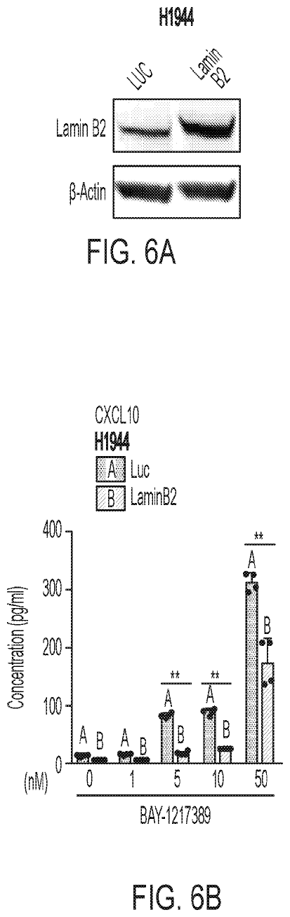

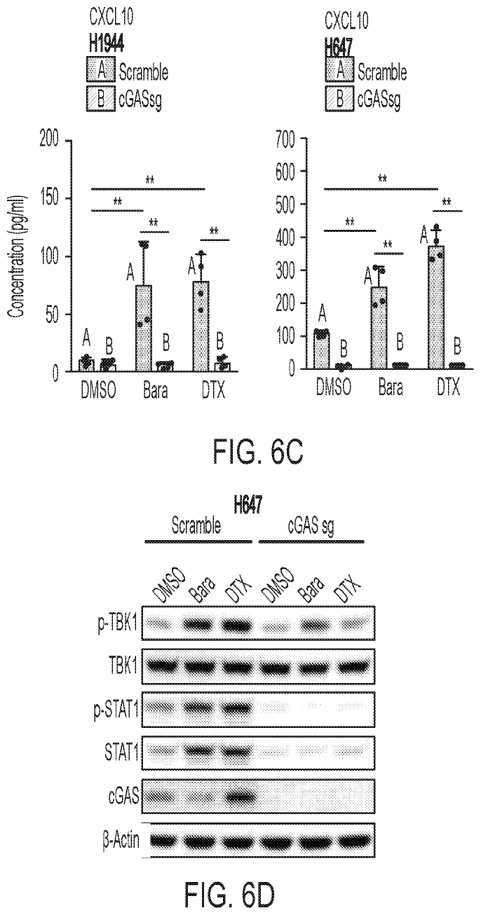

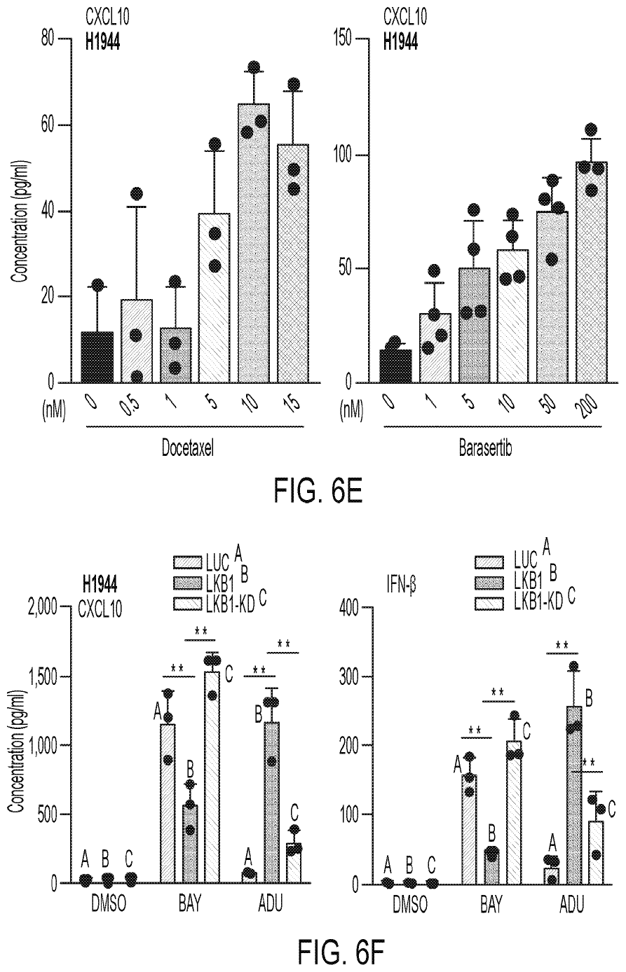

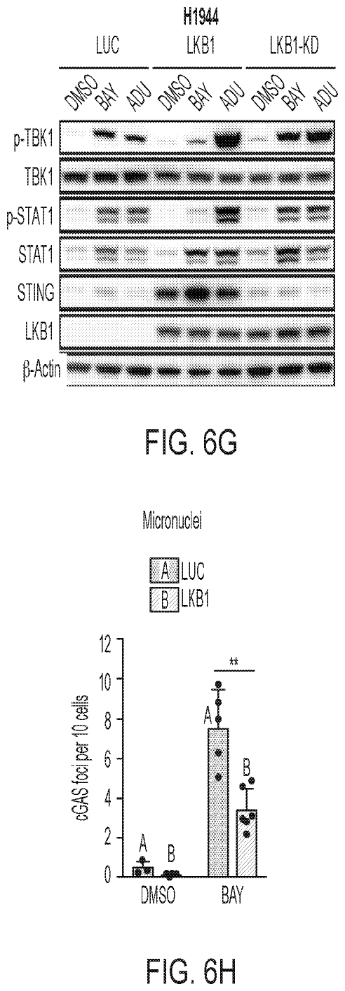

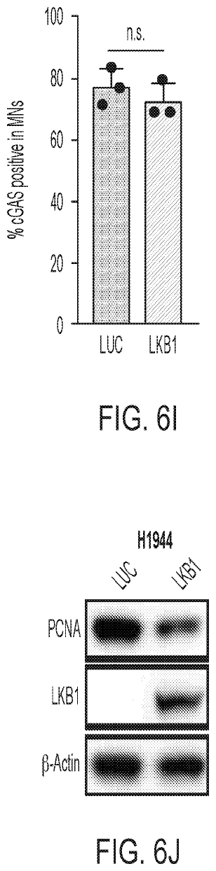

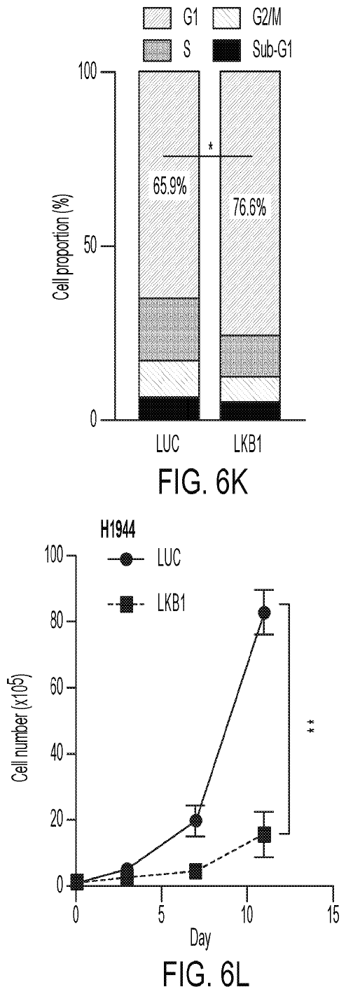

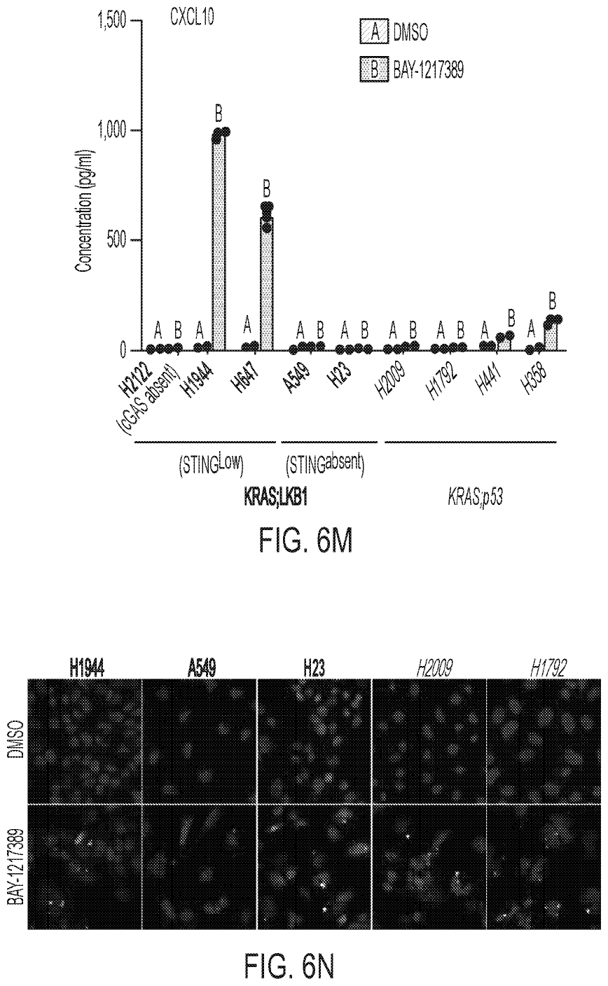

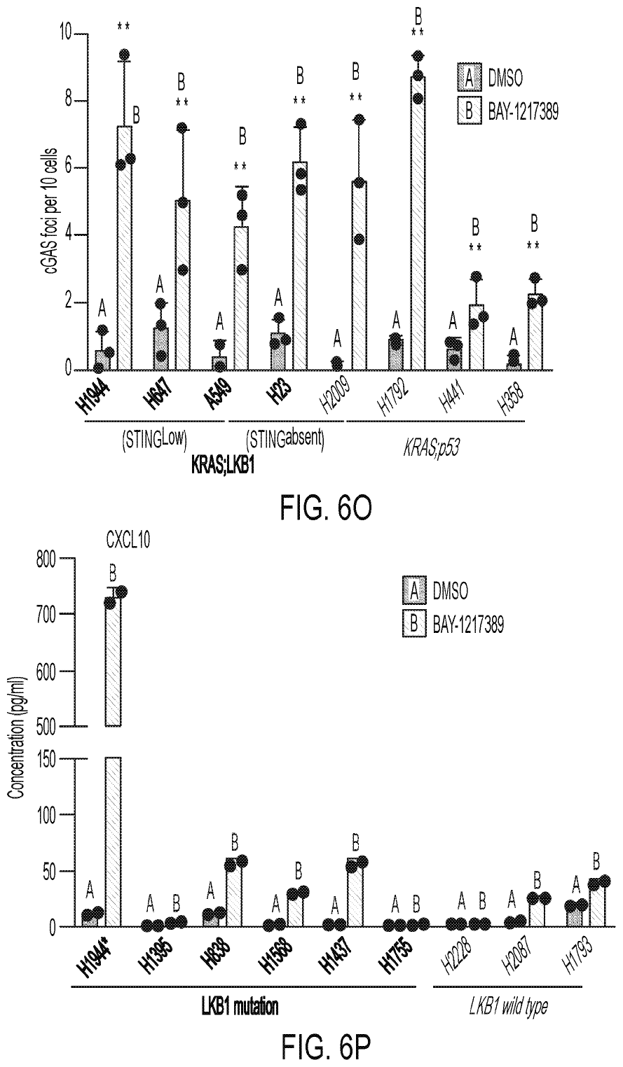

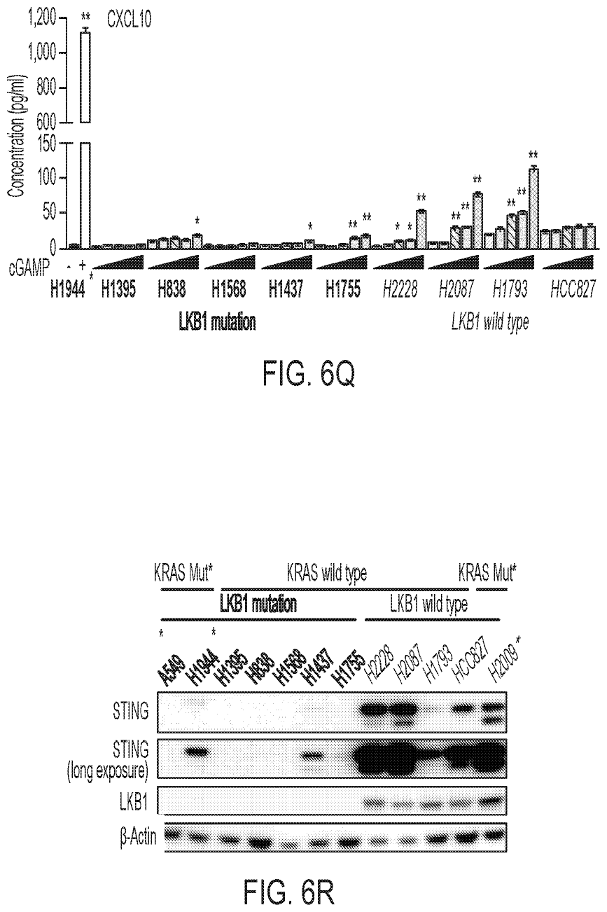

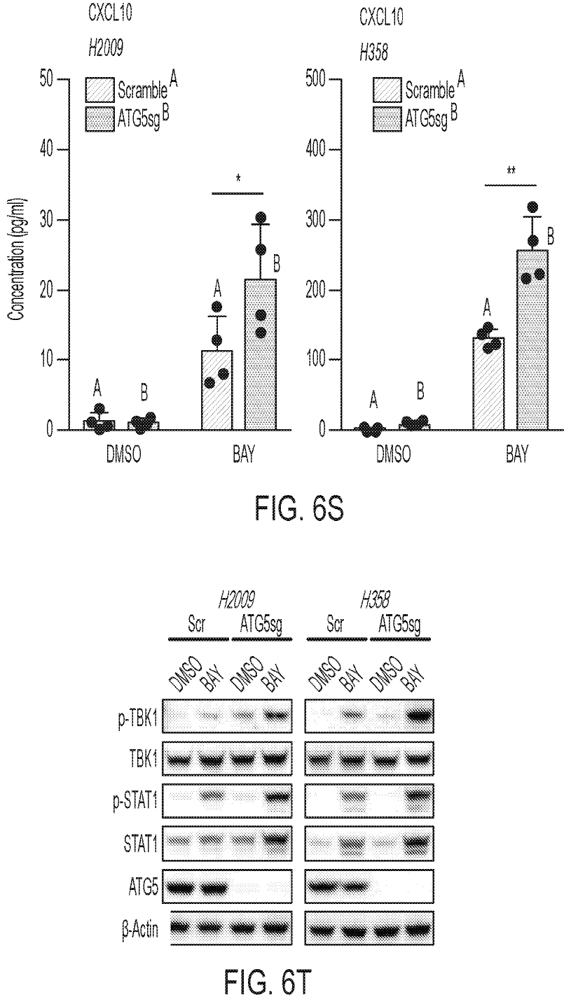

MPS1 is a regulator of the spindle assembly checkpoint (SAC), and MPS1 inhibition in other contexts is known to facilitate massive chromosome missegregation (London and Biggins, 2014). This effect could therefore be related to the formation of micronuclei, known activators of cGAS-STING signaling generated by mitotic slippage and subsequent progression into G1 (Mackenzie et al., 2017). Indeed, treatment of H1944 cells with CFI-402257 using the same 48-hour pulse treatment schedule (FIG. 3B) generated abundant micronuclei, as compared with other drugs, including docetaxel or barasertib (FIGS. 5A and 5B). Furthermore, induction of CXCL10 mRNA expression in H1944 cells directly correlated with the number of micronuclei induced by these DNA-damaging agents, with CFI-402257 representing the clear outlier (FIG. 5C). Additionally, Lamin B2 over-expression, which is sufficient to inhibit micronuclei disruption (Hatch et al., 2013) and could protect against cGAS recognition, attenuated CXCL10 secretion induced by pulse treatment with MPS1 inhibitor (FIGS. 6A and 6B).

Since treatment with docetaxel or barasertib at the IC₅₀ concentrations utilized generated micronuclei, and partially activated CXCL10 production in a cGAS-dependent manner (FIGS. 6C and 6D), it was assessed whether lowering their concentrations could recapitulate the effects of CFI-402257 treatment following pulse treatment. However, CXCL10 induction was not upregulated at lower concentrations of these drugs and, in fact, tended to decrease in a dose-dependent manner (FIG. 6E). These data are consistent with the concept that drugs that induce a potent mitotic arrest do not efficiently create micronuclei, since micronuclei arise when a broken chromosome improperly segregates during mitosis, with subsequent re-entry into G1 phase (Harding et al., 2017). Indeed, in contrast to other DNA damaging agents such as cisplatin or etoposide that strongly induced G2/M-arrest, or pemetrexed or hydroxyurea that induced S-phase arrest, release from CFI-402257 treatment resulted in comparable cell cycle status with control cells, revealing the ability to progress past mitosis (FIG. 5D). Further consistent with this idea, cGAS-STING-induced CXCL10 and IFNβ secretion and activation of STAT1 was substantially weaker during continuous exposure to CFI-402257 over 72 h, as compared with the pulse 48 hour treatment and 24 hour release (FIGS. 5E and 5F). Indeed, in WT-LKB1 reconstituted H1944 cells, which restores STING expression but also impairs cell growth due to its tumor suppressive function, ADU-S100 sensitivity was enhanced while MPS1i impact was dampened, and this required intact LKB1 kinase activity (FIGS. 6F-6L).

Based on these observations, MPS1 inhibition might also induce micronuclei more efficiently in proliferating cancer cells as compared with non-genomically altered, non-proliferative cells in the TME such as immune cells and endothelial cells. Indeed, treatment with CFI-402257 did not significantly activate the STING pathway in terminally differentiated macrophage-like THP1 cells following phorbol 12-myristate13-acetate (PMA) treatment, in contrast to their high sensitivity to 10 μM ADU-S100 treatment (FIGS. 5G and 5H). Conversely, treatment with CFI-402257 more efficiently activated STING signaling in H1944 cells compared to treatment with 10 μM ADU-S100, a concentration that had negligible impact (FIGS. 1A-1B, 5G, and 5H). Collectively, these data reveal a unique property of MPS1 inhibition in activating cGAS-STING signaling in proliferating KL cells as compared with other cells in the TME.

The differential impact of pulse MPS1i across a broader spectrum of lung cancer cell lines was also explored. cGAS was detected by immunofluorescence staining in multiple KL vs KP cell lines, and successfully labeled micronuclei generated following pulse MPS1i treatment, revealing similar numbers regardless of KL or KP status (FIGS. 6M-6O). In consonance with the preferential sensitivity of STING^{Low} KL cells to cGAS-STING activation, substantial CXCL10 induction was observed in H1944 and H647 cells. Pulse MPS1i impact was examined in KRAS WT cells with mutant or intact LKB1 status. While STING expression still tracked with LKB1, strong sensitivity to exogenous 2′3′-cGAMP or MPS1i was not observed (FIGS. 6P-6R), potentially consistent with a recent report demonstrating distinct immune biology of human KL tumors (Ricciuti et al., 2022). Finally, it was assessed whether the defective autophagy present in KL cells could contribute to their particular sensitivity to micronuclei which are eliminated by autophagy (Zhao et al., 2021). Indeed, ATG5 KO increased sensitivity of multiple KP lines to pulse MPS1i (FIGS. 6S and 6T). Thus, pulse MPS1 inhibition potently generates micronuclei, which preferentially activates cGAS-STING signaling in KL cells.

Combination Treatment with MPS1 and Epigenetic Inhibitors Drives Potent STING Activation in KL Cells

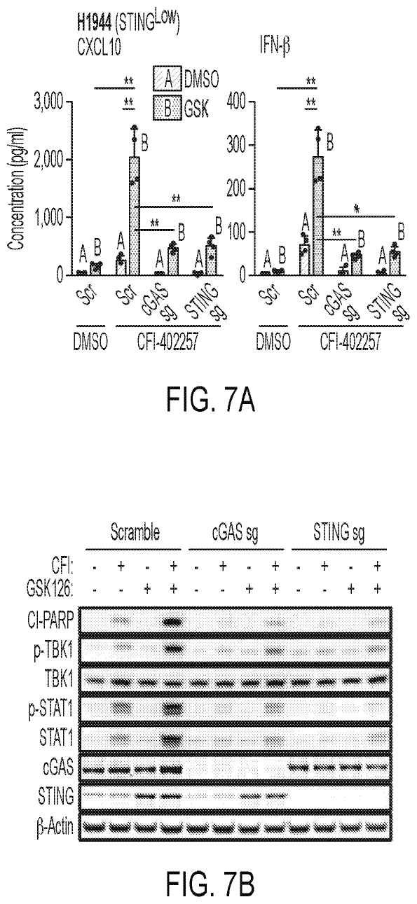

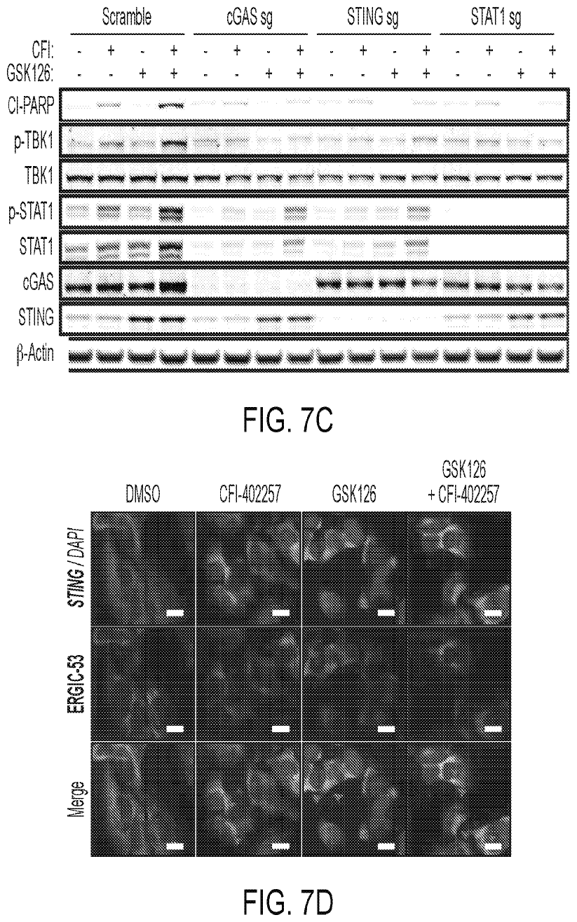

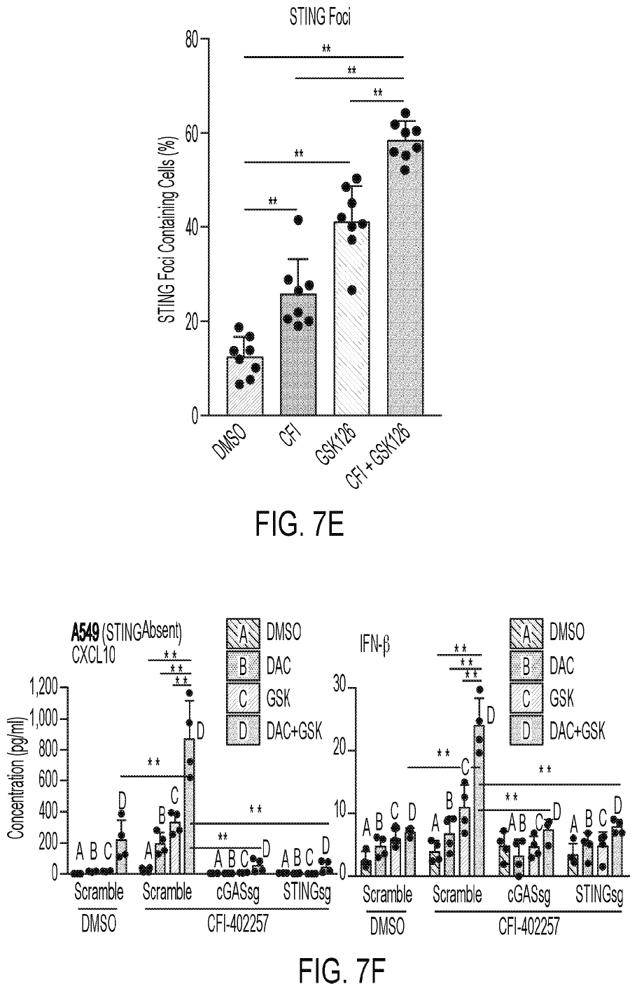

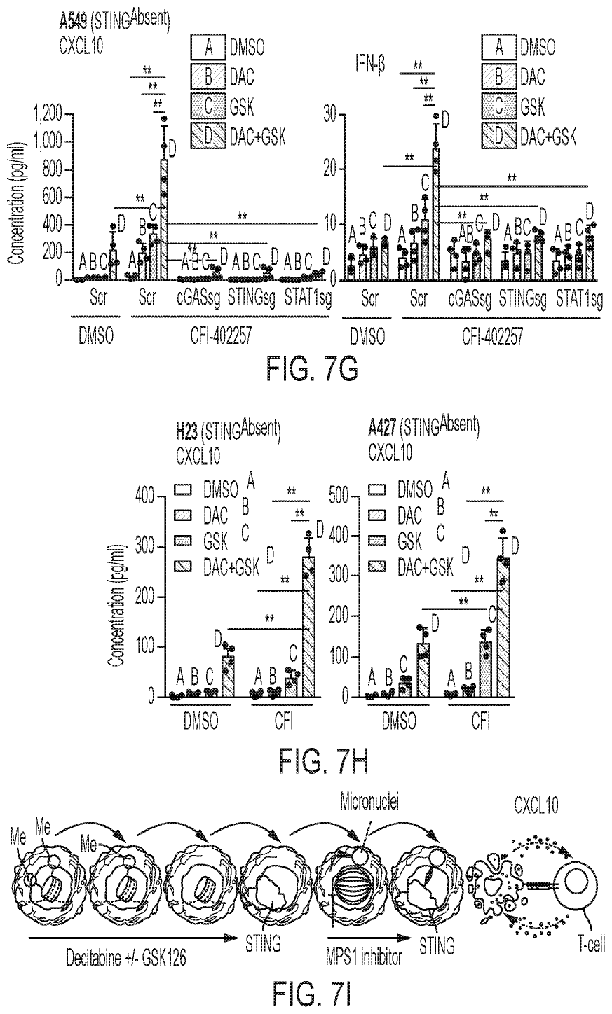

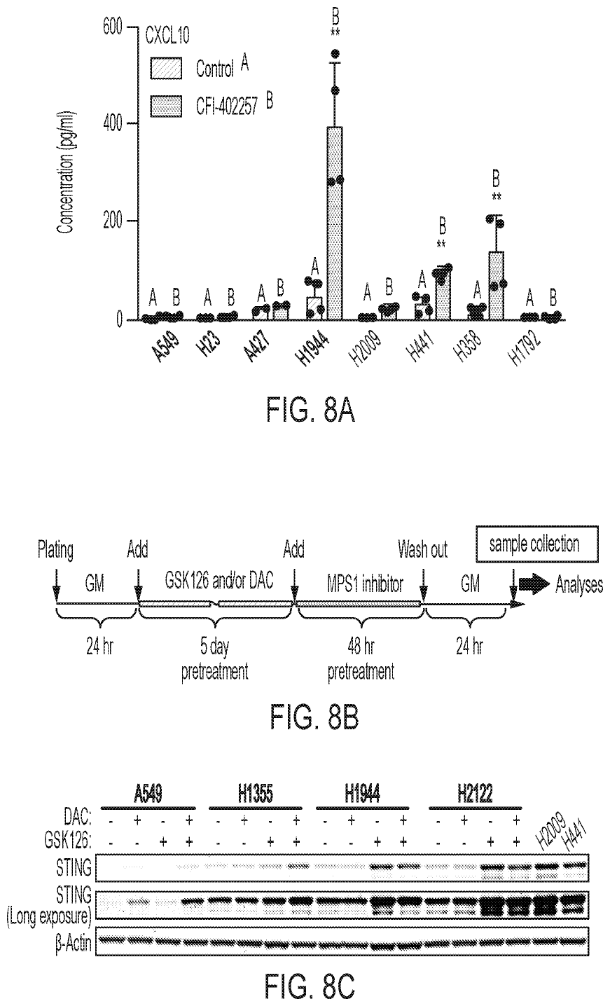

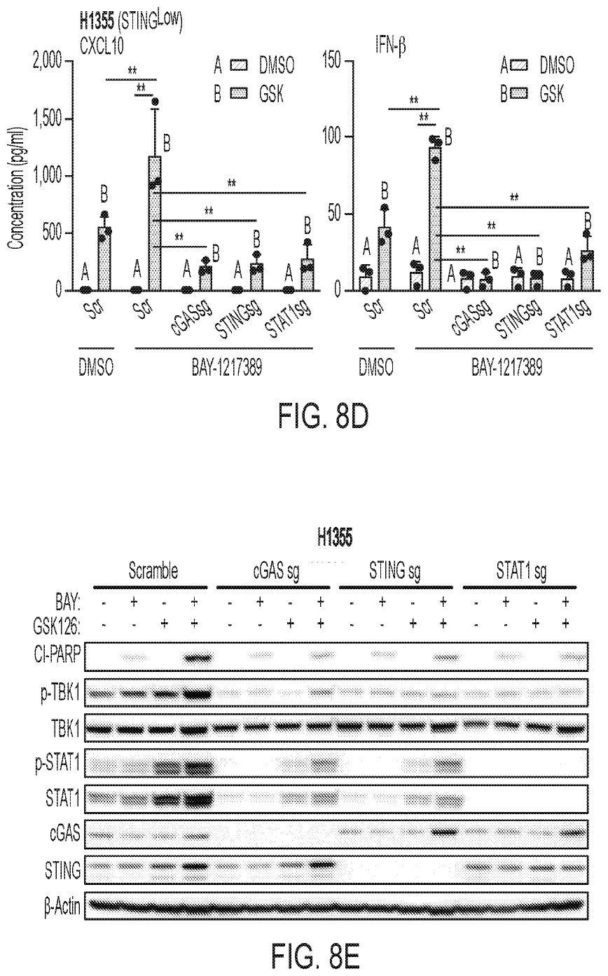

In consonance with their response to exogenous CDNs, H1944 cells still retained higher sensitivity to CFI-402257 as compared with KP cell lines, even in spite of their suppressed STING expression (FIGS. 8A and 8B). However, since KL cells epigenetically silence STING, unleashing STING expression might robustly sensitize them to MPS1 inhibition, including cell lines with baseline STING absence. Pre-treatment with the EZH2 inhibitor GSK126, which is able to de-repress STING in STING^{Low} KL cells lines, H1944, and H1355 cells, was explored (Kitajima et al., 2019) (FIGS. 8B and 8C). Pre-treatment of H1944 or H1355 cells with GSK126 markedly enhanced CXCL10 and IFN-β secretion induced by pulse MPS1i treatment, which remained dependent on intact cGAS, STING, or STAT1 (FIGS. 7A, 8D). Furthermore, combination therapy with GSK126 followed by MPS1i treatment synergized to induce potent TBK1 and STAT1 activation, as well as PARP cleavage, which was similarly blocked by deletion of cGAS, STING, or STAT1 (FIGS. 7B, 7C, and 8E). GSK126 combination therapy not only upregulated STING levels, but also increased STING colocalization with the ER-Golgi intermediate compartment (ERGIC), indicative of translocation to its active state where it complexes with TBK1 to induce downstream signaling (FIGS. 7D and 7E).

In contrast to STING^{Low} H1944 cells, STING downstream signaling was not upregulated following MPS1i treatment in STING^{Absent} KL cells (FIG. 6K). Previously, it was reported that superimposed DNA hypermethylation of the promoter region of STING is a key epigenetic modification to abolish STING expression in STING^{Absent} KL cells (Kitajima et al., 2019). The consequences of MPS1i following treatment of these cell lines with the DNMT inhibitor decitabine (DAC) alone or in combination with GSK126 to restore STING expression were examined. Remarkably, A549 cells, which were almost completely resistant to CFI-402257 treatment alone, exhibited potent induction of CXCL10 and IFNβ secretion following DAC −/+GSK126 treatment (FIG. 7F, 7G, and 8C). This effect was directly related to restoration of cGAS-STING signaling, since deletion of cGAS, STING, or STAT1 completely suppressed secretion of CXCL10 and IFNβ induced by these treatments (FIG. 7G). Furthermore, it was determined that DAC and GSK126 treatment primed response to MPS1i across multiple additional STING^{Absent} KL cell lines (FIG. 7H). Taken together, these data reveal that de-repressing STING by targeting its epigenetic silencing markedly enhances the impact of MPS1i in KL cells, co-opting their de-regulated cell cycle progression and potently restoring CXCL10 production and type I IFN signaling (FIG. 7I).

### MPS1 Inhibition Upregulates HLA Expression in KL Cells and Enhances Immune Cell Chemotaxis

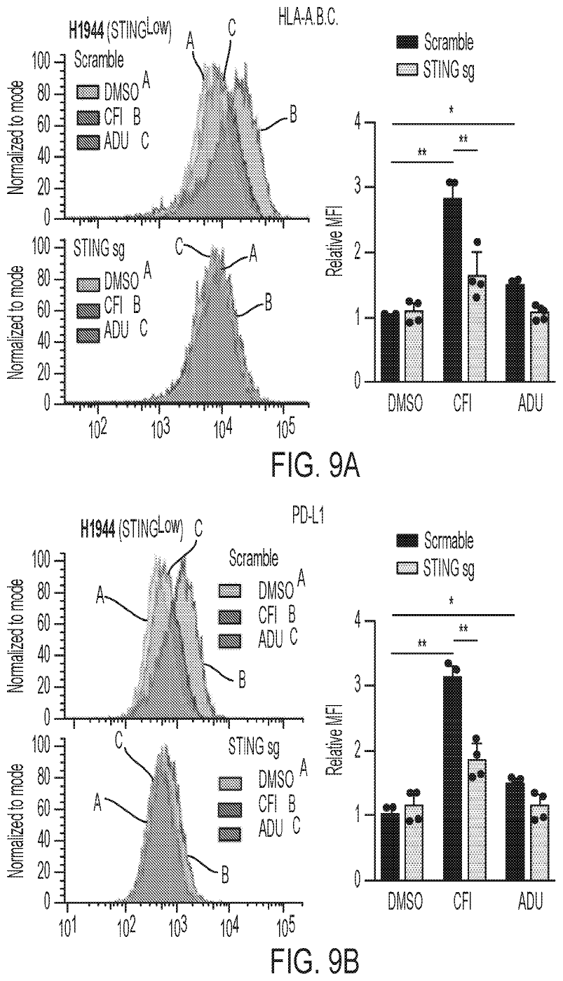

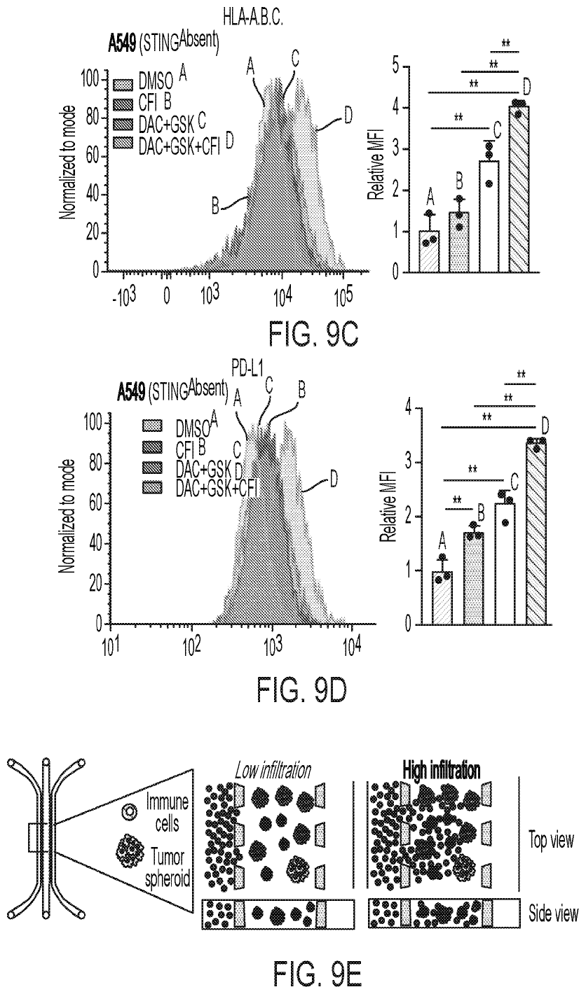

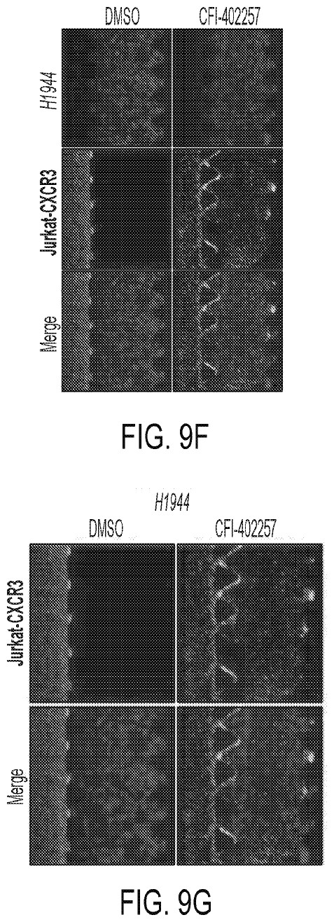

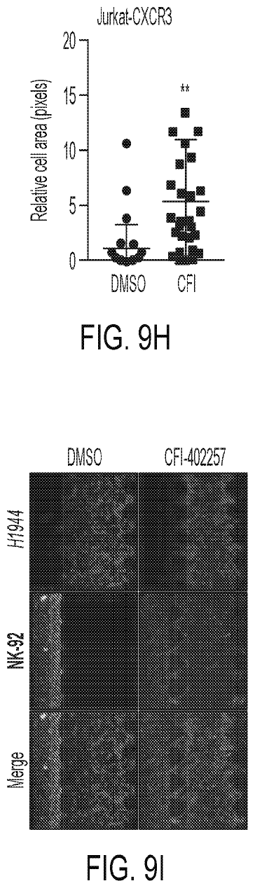

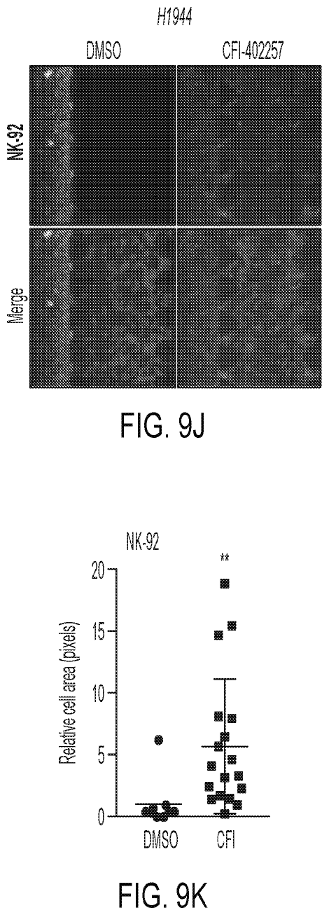

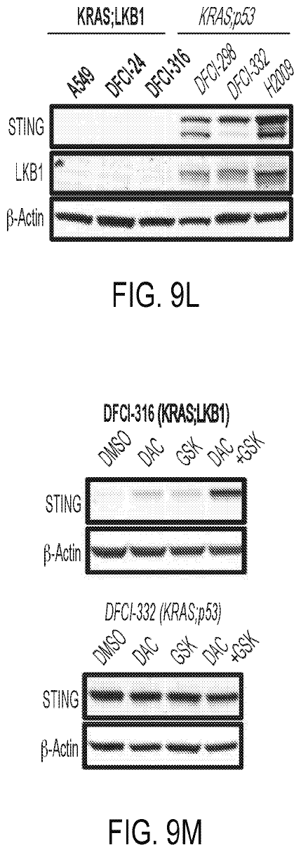

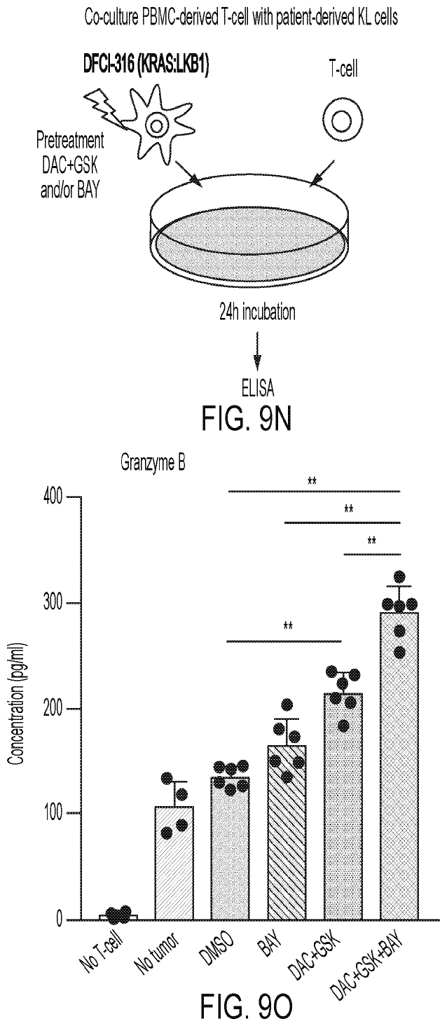

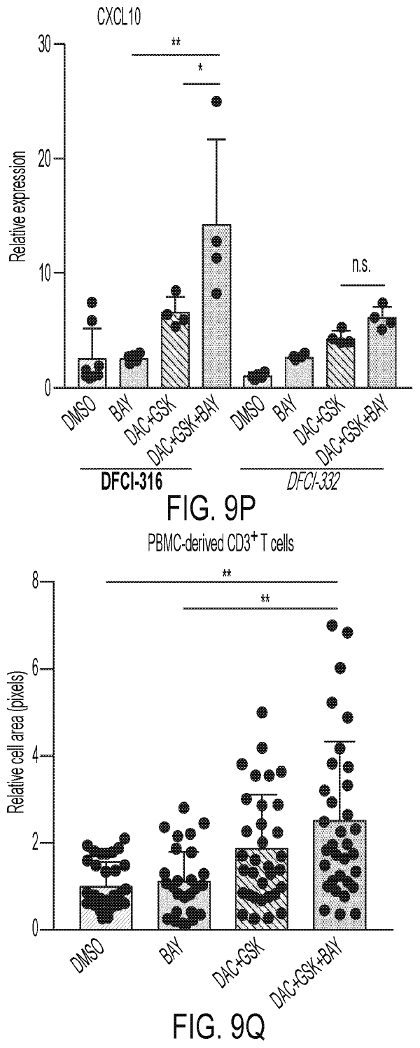

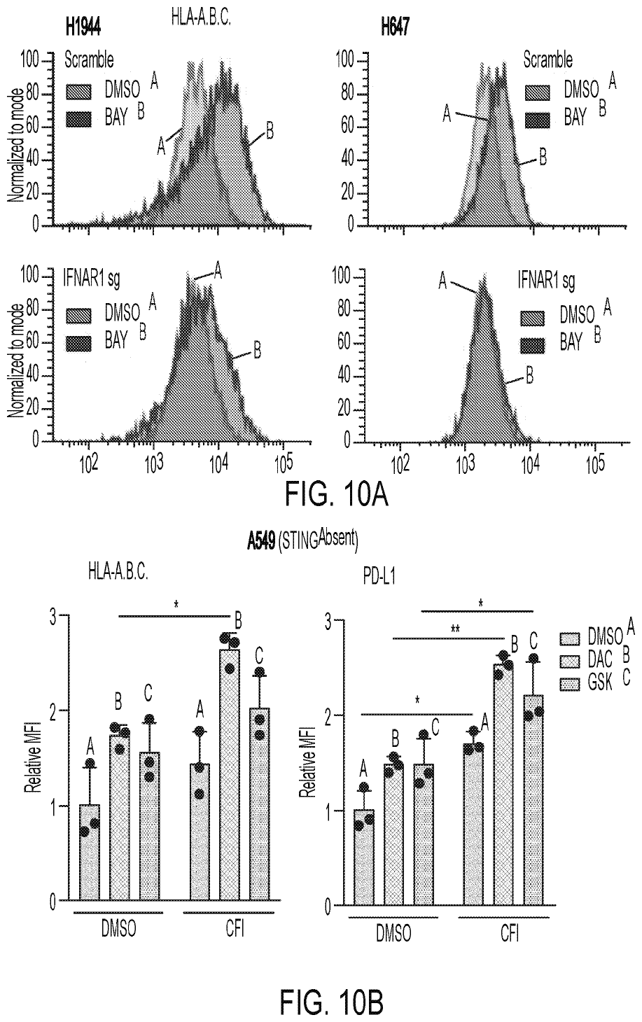

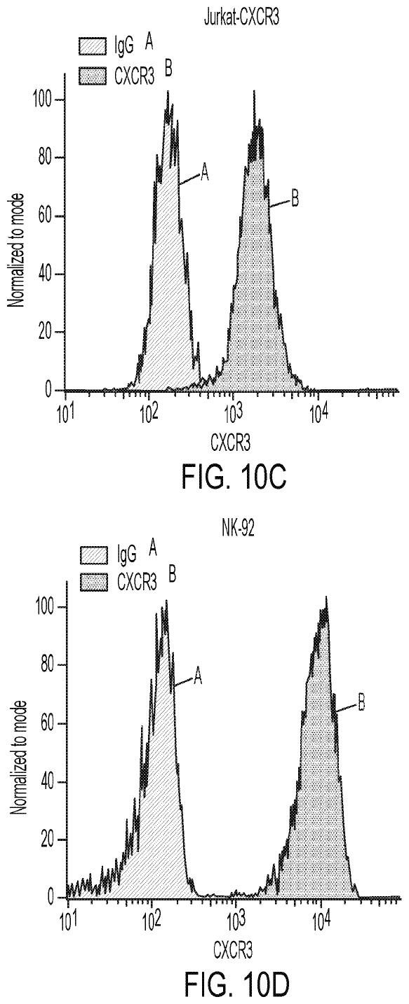

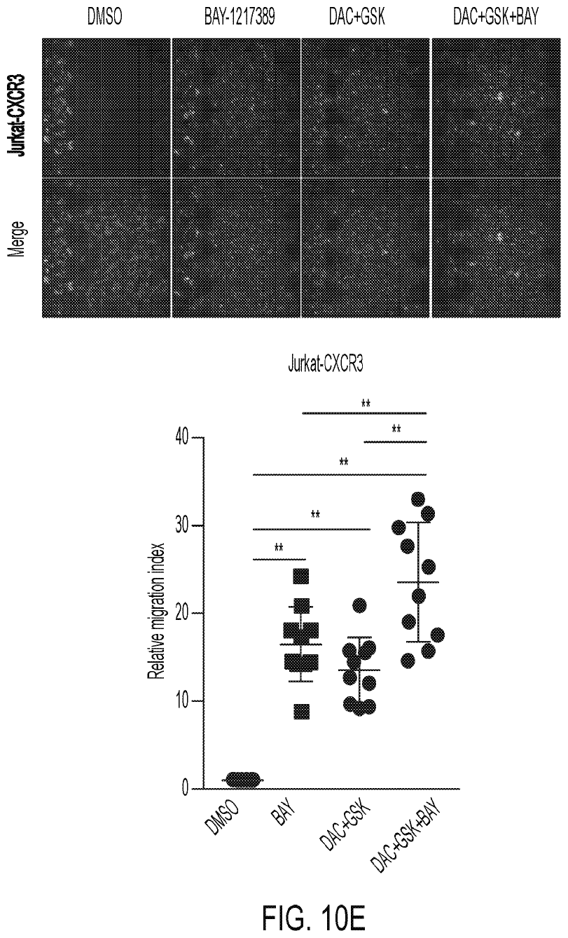

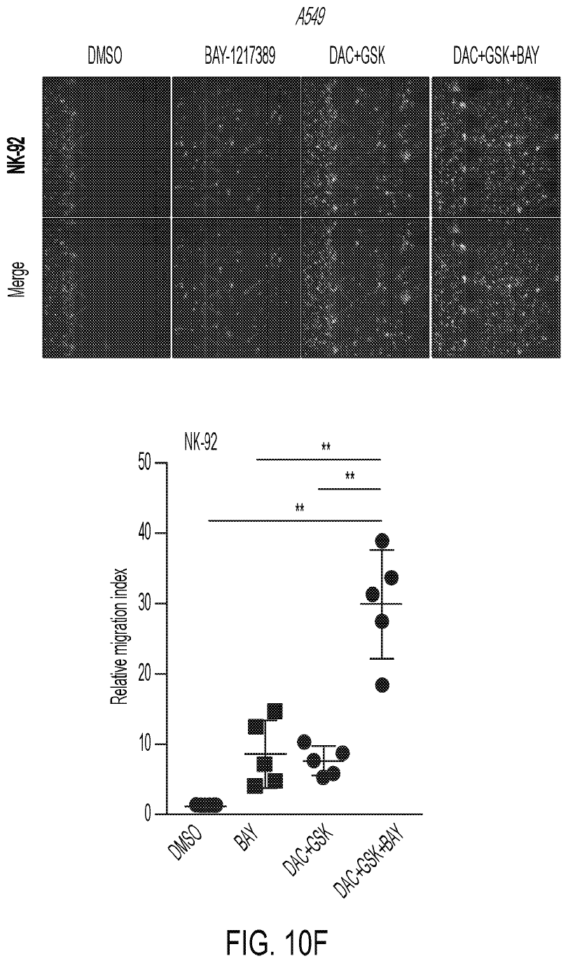

Re-activation of cGAS-STING signaling and CXCL10 in KL cells promotes T-cell extravasation from the vasculature and intra-tumoral T cell recruitment (Campisi et al., 2020; Kitajima et al., 2019). Furthermore, downregulation of MHC class I, also a STAT1 target, has been implicated in KL cell immune evasion (Deng et al., 2021). The surface expression of MHC class I molecules HLA-A.B.C in H1944 cells was examined to test the potential impact of MPS1 inhibitor induced STING activation on antitumor immunity. As compared with exogenous ADU-S100 treatment, intracellular 2′3′-cGAMP induction via pulse MPS1i significantly induced the expression of MHC class I (FIG. 9A). STING dependence of this phenomenon was also shown because STING or IFNAR1 depletion abrogated the impact of MPS1i on MHC class I induction (FIGS. 9A, 10A). KL cells are also known to maintain low levels of PD-L1 expression (Koyama et al., 2016; Skoulidis et al., 2018). Similarly, it was observed that pulse MPS1i also induced PD-L1 expression on the cell surface, which was suppressed by STING depletion (FIG. 9B). Next, it was examined whether restoration of STING expression in A549 STING^{Absent} KL cells might also promote MPS1 inhibitor mediated induction of MHC class I. Consistent with its impact on STING upregulation (FIG. 8C), it was observed that pretreatment with DAC −/+GSK126 upregulated both MHC class I and PD-L1 expression in response to CFI-402257 treatment, with combined DAC and GSK126 pretreatment priming the most potent effect (FIGS. 9C, 9D, and 10B). Taken together, these data reveal that MPS1 inhibitor-induced activation of STING can restore MHC class I expression, as well as PD-L1, on both STING^{Low} and STING^{Absent} KL cells after epigenetic priming.

Next, immune cell chemotaxis following MPS1 inhibition was investigated using a previously described three-dimensional (3D) microfluidic system (FIG. 9E) (Kitajima et al., 2019; Ritter et al., 2020). H1944 KL cells were treated with CFI-402257 or DMSO control in two-dimensional culture, and tumor spheroids were formed using an ultra-low attachment plate, and then embedded in collagen into the central channel of a 3D microfluidic device. As expected, CXCR3-reconstituted Jurkat T-cell (FIG. 10C) migration was only observed in the presence of H1944 KL cells that were pretreated with CFI-402257 (FIGS. 9F, and 9G-9H), consistent with their enhanced CXCL10 production (FIG. 2F). In addition, NK-92 cells, which endogenously express CXCR3, were also utilized (FIG. 10D) for this immune cell migration assay. Similarly, it was observed that treatment with CFI-402257 also accelerated the migration of NK-92 cells from the side channel towards tumor cell spheroids (FIGS. 9J-9K). In addition, consistent with restoration of STING expression and subsequent activation of STING downstream, it was observed that pretreatment with DAC −/+GSK126 enhanced the migration of both Jurkat-CXCR3 and NK-92 cells induced by MPS1i treatment in A549 STING^{Absent} KL cells (FIGS. 10E and 10F).

Next, it was assessed whether MPS1i treatment upregulates antigen-presentation and immune cell recruitment in models more closely obtained from patients. Multiple patient-derived xenograft (PDX) cell lines were examined, and low to absent STING expression in KL cells similar to A549 STING^{Absent} KL cells was confirmed, which was restored with DAC+GSK126 treatment (FIGS. 9L and 9M). Conversely, patient-derived KP cells retained STING expression, and treatment of epigenetic inhibitors did not upregulate STING expression (FIGS. 9L and 9M). Indeed, pretreatment of patient-derived KL cells with epigenetic and pulse MPS inhibition, and then co-culture with allogeneic T cells derived from peripheral blood mononuclear cells (PBMCs) reveals significantly enhanced granzyme B induction particularly, revealing increased immunogenicity (FIGS. 9N and 9O). Moreover, this same pretreatment promoted CXCL10 secretion and migration of PBMC-derived CD3+ T cells into PDX KL spheroids in 3D culture (FIGS. 9I, 9P, and 9Q). Taken together, these results reveal that epigenetic and pulse MPS1 inhibitor treatment in KL cells can improve antigen-presentation and T/NK cell recruitment.

### Sequential DNMT/MPS1 Inhibitor Pulse Treatment Induces Durable Therapeutic Efficacy in a Syngeneic Murine KL Model In Vivo

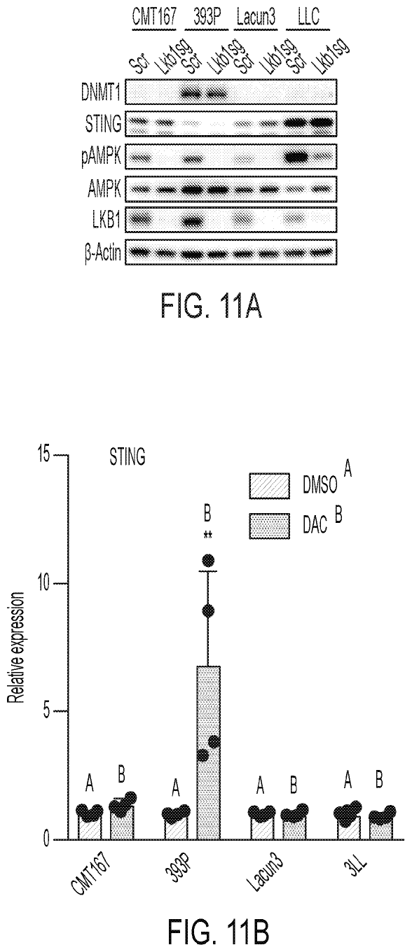

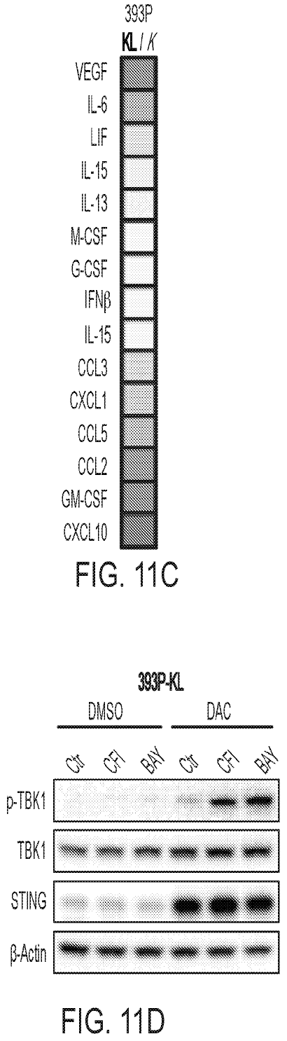

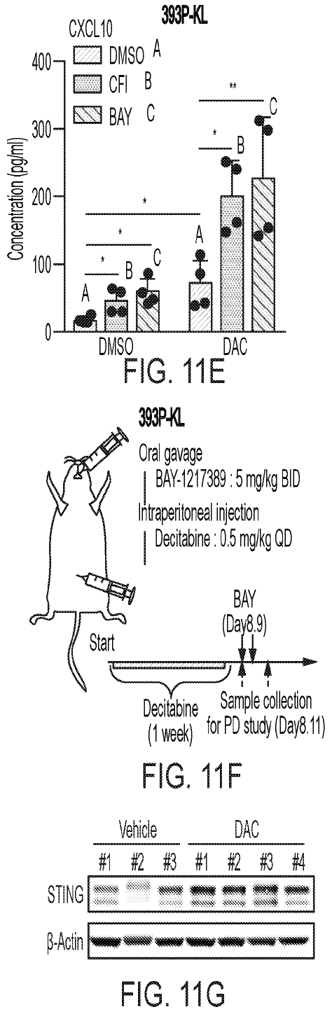

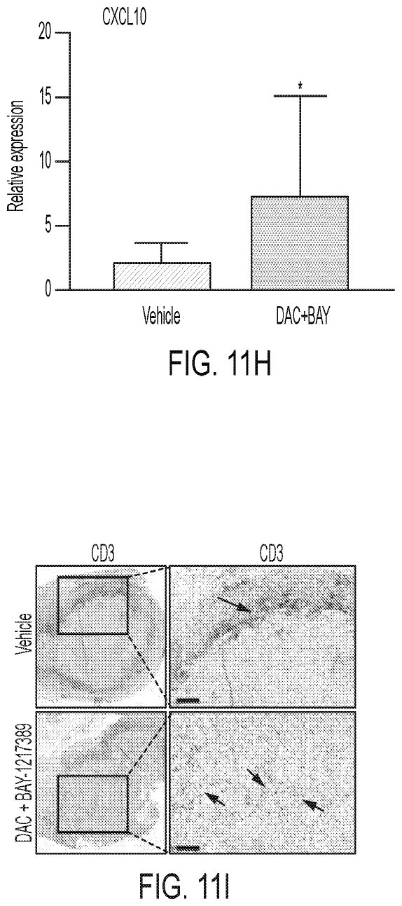

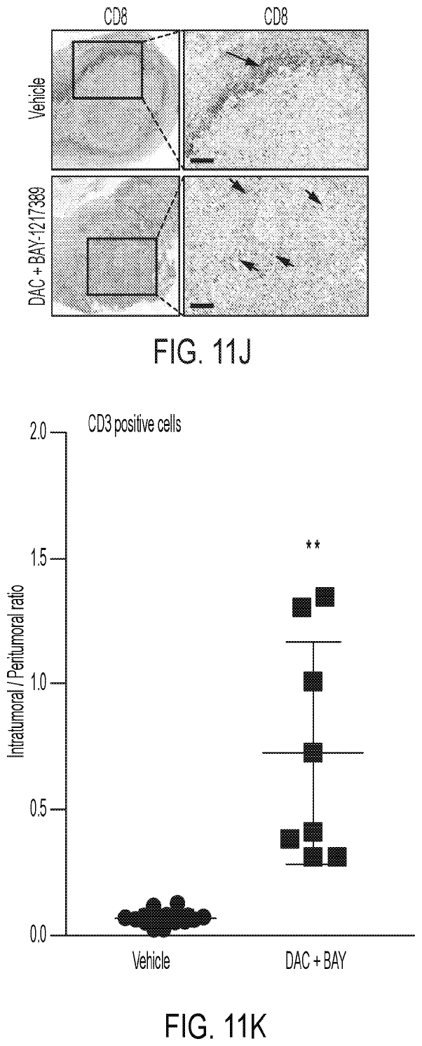

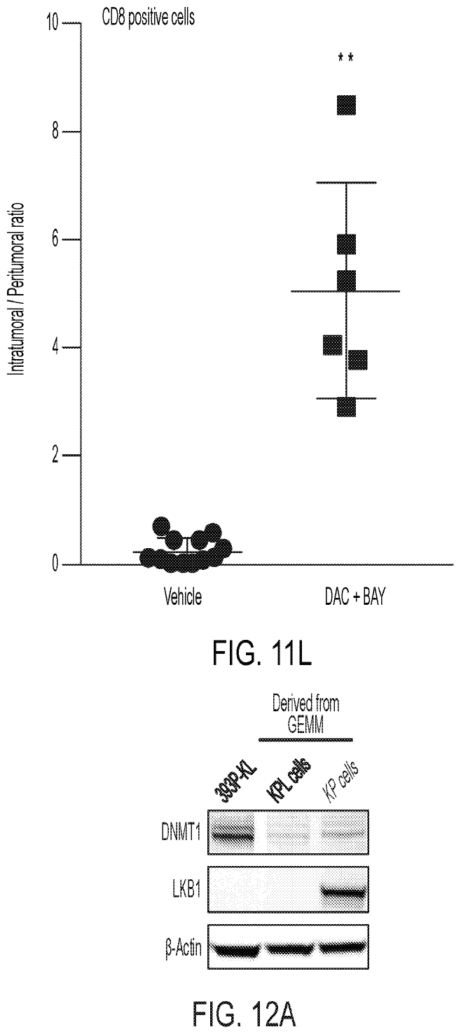

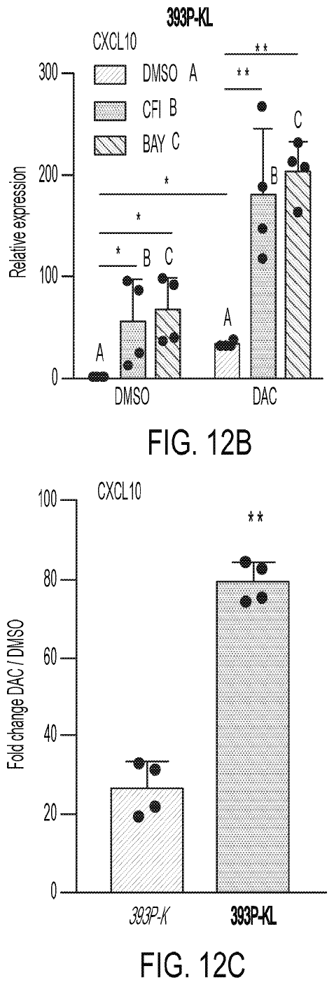

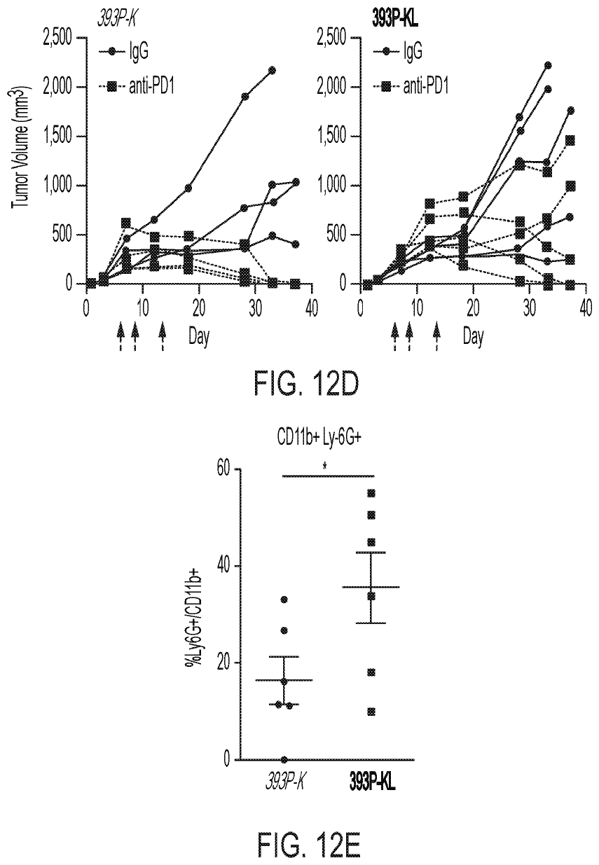

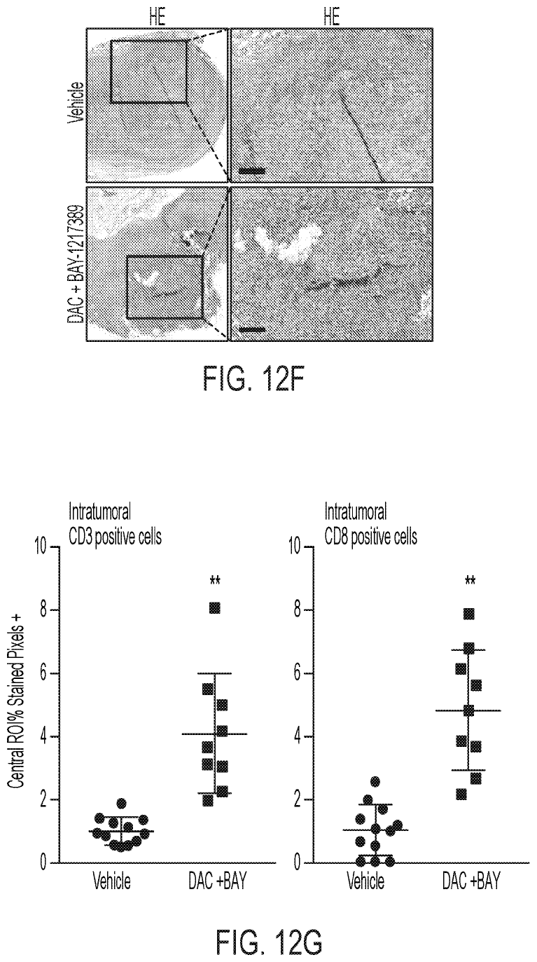

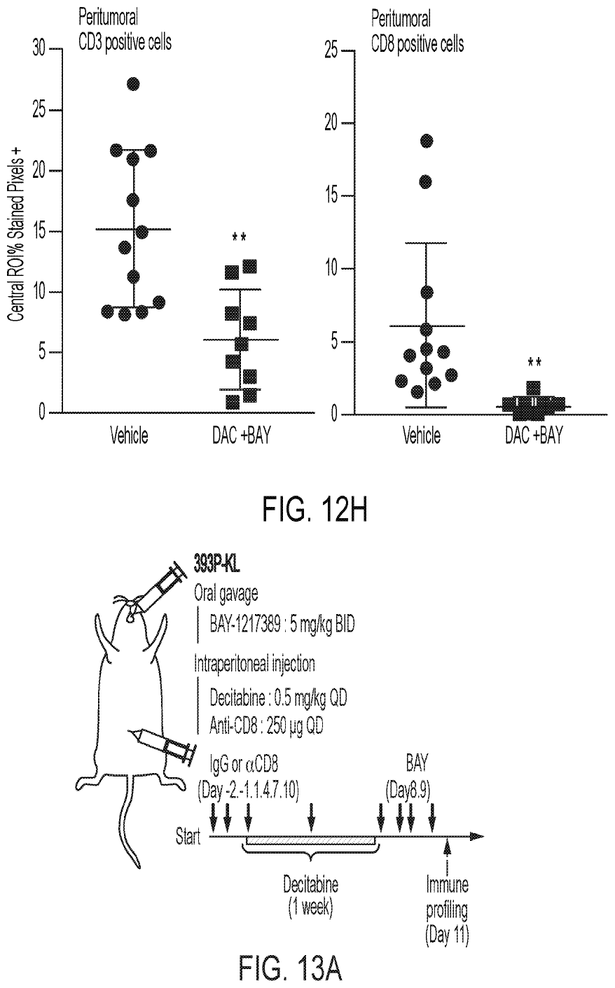

Since re-activation of STING signaling by MPS inhibition in KL cells might thus promote antitumor immunity as well as intrinsic cell death, a syngeneic murine KL model to examine therapeutic consequences in vivo was established. As the process of immune editing in naturally arising human lung cancers versus engineered mouse tumors is vastly different, multiple murine Kras mutant lung cancer models derived from CMT167, 393P, Lacun3, and LLC1 cells were characterized to select a model that most closely recapitulates the biological features of human KL lung cancers. Among these cell lines, and in contrast to those derived from poorly immunogenic genetically engineered mouse models (GEMMs) (Koyama et al., 2016), it was observed that 393P cells naturally upregulate Dnmtl to suppress STING, and that LKB1 inactivation further abrogated Sting expression in this model, inverting cytokine secretion to promote IL-6 release (FIGS. 11A-11C, and 12A). Indeed, pretreatment of 393P-KL cells with DAC not only restored Sting protein levels, but also uniquely promoted high CXCL10 expression and MPS1i induced Sting signaling (FIGS. 11D, 11E, 12B, and 12C). Furthermore, whereas parental 393P-K cells were immunogenic and responsive to PD-1 blockade, 393P-KL cells exhibited relative anti-PD-1 resistance (FIG. 12D), and increased infiltration of CD11b+Ly-6G+ granulocytes in the TME, all consistent with their baseline 11-6 upregulation and modeling of in vivo KL immunobiology (FIG. 12E) (Koyama et al., 2016; Skoulidis et al., 2018).

Next, using this syngeneic 393P-KL model, a pulse schedule of combination therapy with DAC and MPS1 inhibition was examined, using the clinically relevant compound BAY-1217389, because of its well-established in vivo dosing (Maia et al., 2018; Wengner et al., 2016). Initially, in a pharmacodynamic study, 393P-KL tumors in the syngeneic 129S2/SvPasCrl mice were treated daily with 0.5 mg/kg DAC for 7 days, and tumors were harvested 24 hours after the last dose to check for restoration of STING in vivo (FIG. 11F). As observed in the in vitro study, it was shown by immunoblot that STING expression was increased following DAC treatment versus vehicle control (FIG. 11G). Next, the impact of subsequent pulse treatment with twice daily 5 mg/kg BAY-1217389 for 2 days was assessed. DAC treatment followed by BAY-1217389 significantly increased Cxcl10 expression by mRNA in bulk tumors (FIGS. 11F and 11H). Examination of vehicle or combination treated tumors by IHC for total CD3+ and CD8+ T cells revealed pronounced T cell exclusion of control tumors, in further agreement with their STING silencing and the phenotype of KL human tumors (FIGS. 11I-11L and 12F-12H) (Kitajima et al., 2019; Skoulidis et al., 2018). As expected, restoration of STING and CXCL10 expression following DAC/MPS1i treatment promoted redistribution of CD3+ and specifically CD8+ T cells from the tumor periphery to the tumor interior, suggesting that this combination could potentially restore tumor immunogenicity in vivo (FIGS. 11F, 11I-11L and 12F-12H).

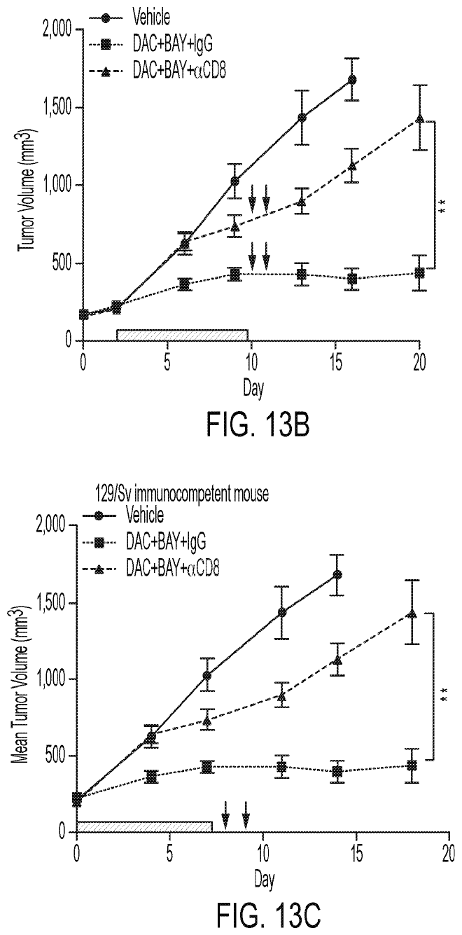

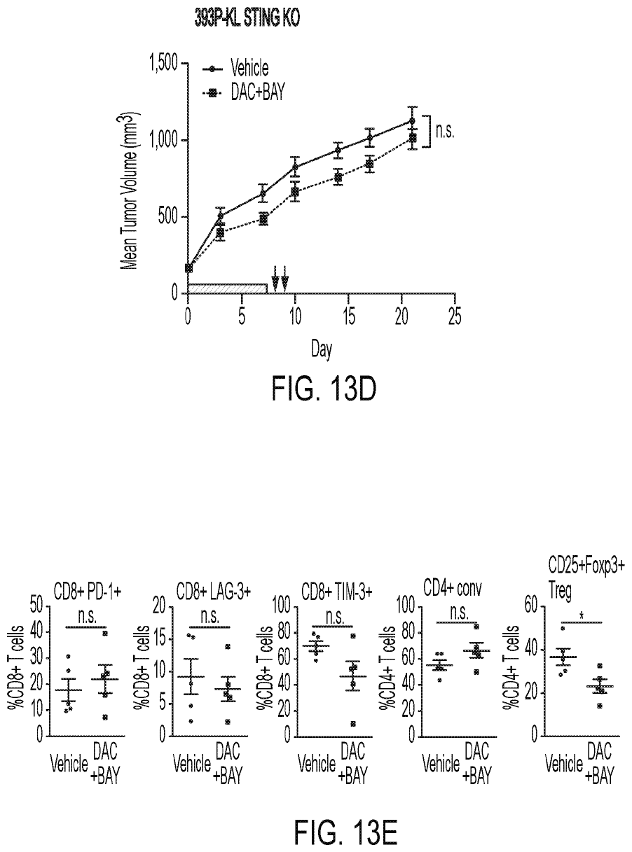

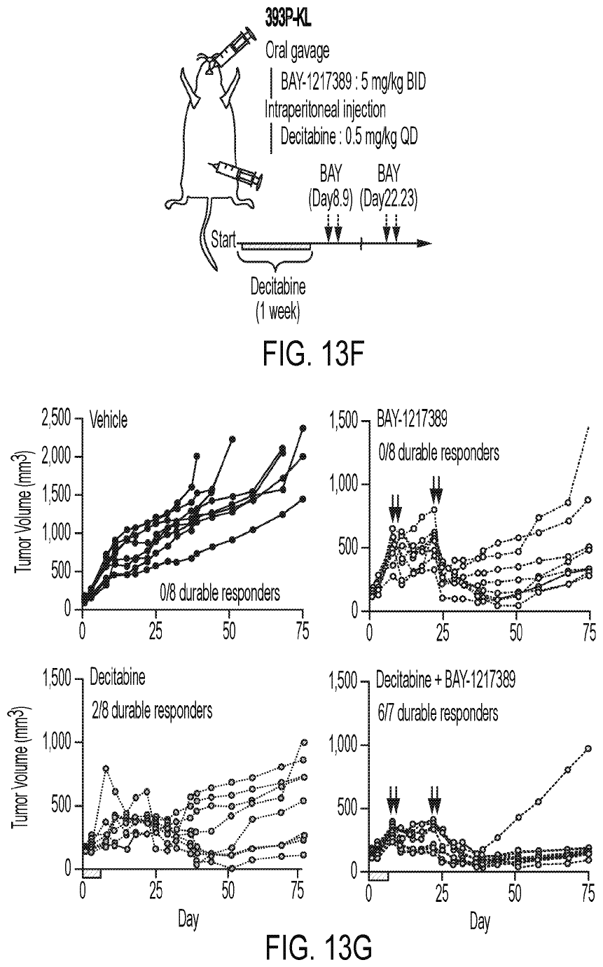

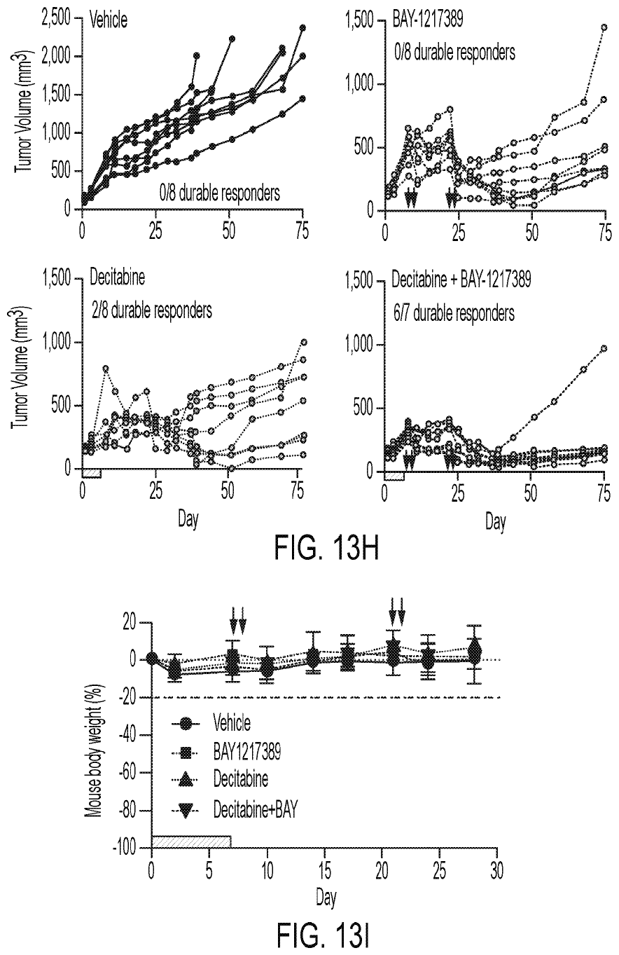

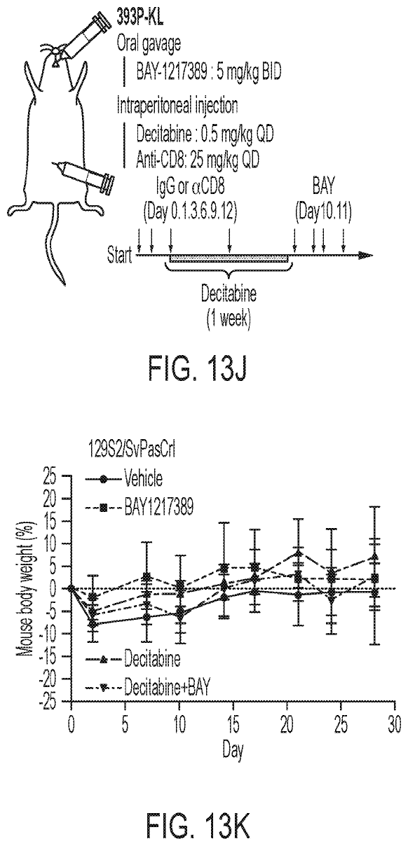

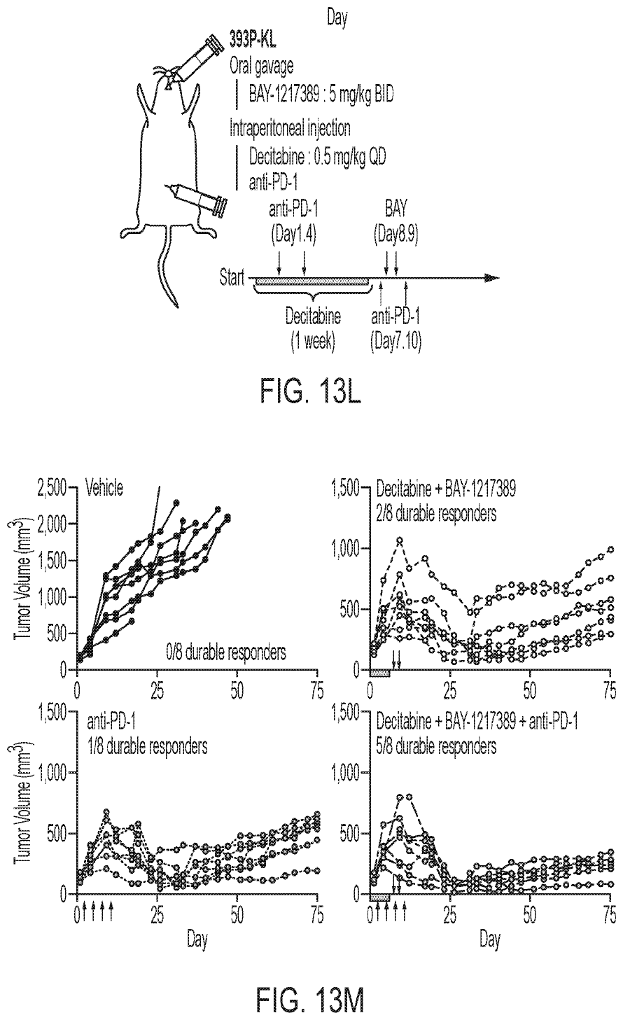

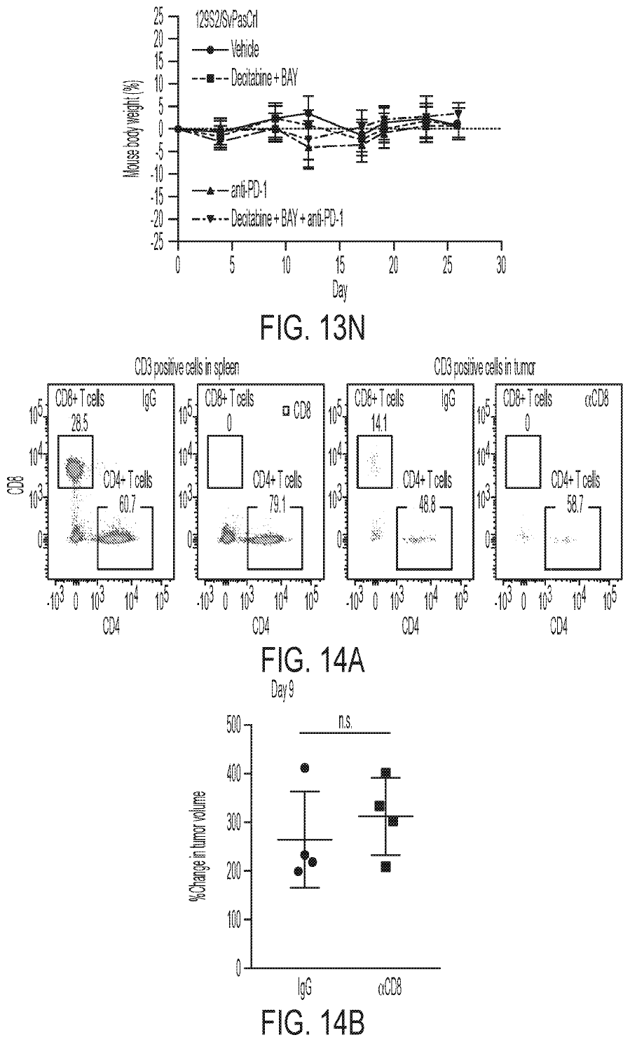

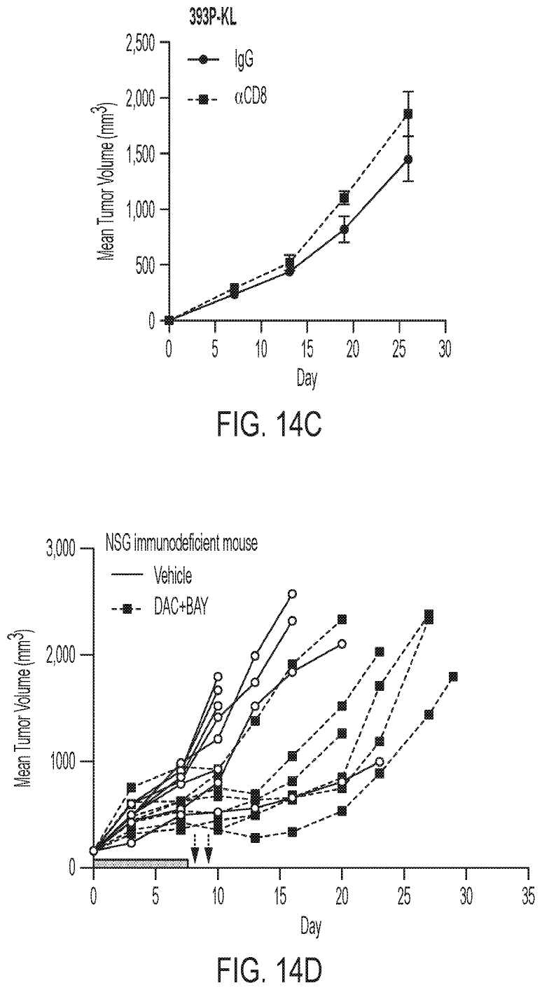

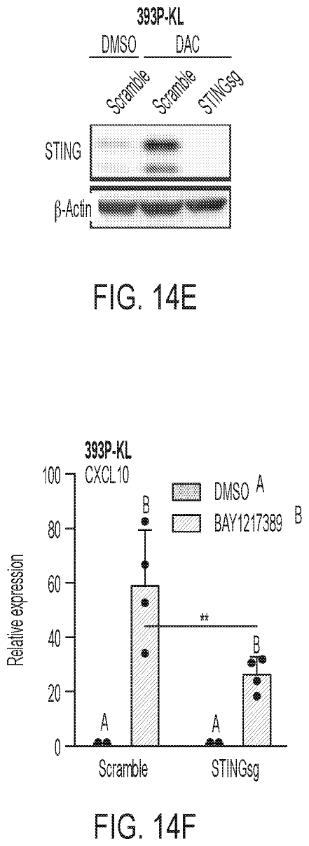

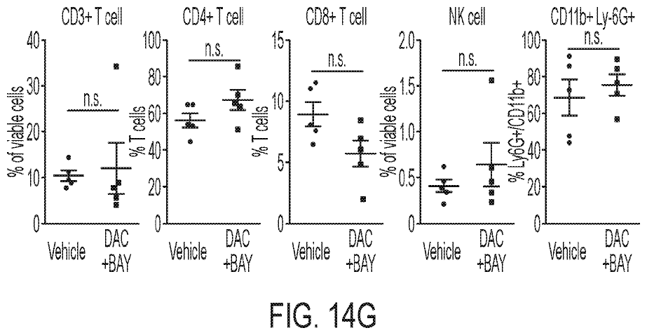

To test this, an efficacy study was performed utilizing the same short-term pulse regimen of the sequential therapy with DAC/MPS1i in 393P-KL syngeneic model. This treatment induced potent inhibition of tumor growth (FIGS. 13A and 13C). To directly assess the role of CD8+ T cell redistribution in mediating this therapeutic effect, the effects of CD8+ depletion was tested using a CD8 depleting antibody (FIG. 13A). Consistent with a key role for CD8+ T cell immunity, the impact of sequential therapy of DAC and BAY-1217389 was significantly attenuated by CD8+ T cell depletion (FIGS. 13B, 13C, 14A, and 14C). Additionally, to further assess the role of an intact immune system, immune-deficient NSG mice were used. The 393P-KL tumors grew quite robustly in NSG mice, and sequential treatment with DAC/MPS1i resulted in only partial tumor growth inhibition with aggressive rebound within 8 days after stopping treatment (FIG. 14D). Next, to directly investigate the dependency of this response on activation of tumor cell STING, STING depleted 393P-KL cells were established, and then the efficacy of pulse combination therapy was evaluated (FIG. 14E). STING depleted 393P-KL showed impaired CXCL10 response following MPS1i treatment in vitro (FIG. 14F), which translated to inactivity of DAC/MPS1i treatment in vivo (FIG. 13D).

To profile the immune response that develops following sequential decitabine/MPS1i combination therapy in greater depth, comprehensive immune profiling by flow cytometry 48 hours post-treatment was performed (FIG. 13A). At this early timepoint, a significant change in absolute T, NK, or myeloid cell numbers was not observed (FIG. 14G). Assessment of CD8+ T cell activation/exhaustion markers such as LAG-3, TIM-3, and PD-1 were not significantly impacted; however, an alteration in CD4+ T cell subsets with significant depletion of CD25+ Foxp3+ Tregs was observed (FIG. 13E). These results are consistent with prior observations that type I interferon signaling can impair Tregs (Gangaplara et al., 2018), revealing that restoration of this tumor cell STING-IFN-CXCL10 axis by this sequential combination therapy promotes both Treg depletion and CD8+ T cell infiltration in vivo.

Next, two different longer-term efficacy therapeutic strategies were assessed, to explore the impact on durable response in these models. The impact of adding either a second MPS1i pulse after 2 weeks, or combination of the single pulse with PD-1 blockade was assessed. Following just a second MPS1i pulse, a durable, long-term response in 6/7 mice after 12 weeks with the combination therapy was observed, in contrast to 2/8 mice treated with DAC alone and 0 mice treated with BAY-1217389 or vehicle alone (p<0.01, Chi-square test) (FIGS. 13F-13H). Of note, treatment of immune-competent mice with this pulse combination therapy schedule was also very well-tolerated, without any evidence of distress or body weight loss (FIGS. 13I and 13K). To directly assess the role of cytotoxic T cell redistribution in mediating this therapeutic effect, CD8+ T cells were depleted using a CD8-specific neutralization antibody (FIG. 13J). While CD8+ T cell depletion did not impact the baseline growth of 393P-KL tumors in vivo (FIG. 14B), the efficacy of sequential combination therapy with DAC and BAY-1217389 was attenuated in CD8+ T cell depleted mice as compared with isotype control treated animals (FIG. 13B). Finally, it was also observed that pulse combination therapy with DAC and BAY-1217389 together with anti-PD1 treatment was tolerable and resulted in increased durable long-term responses and significant tumor growth suppression as compared to either arm alone (p<0.05, Chi-square test) (FIGS. 13L-13N). In sum, these data reveal that sequential combination therapy with DAC and pulse BAY-1217389 treatment can restore durable response in STING-silenced KL tumors and also improve sensitivity to PD-1 blockade.

### DISCUSSION

LKB1 mutation has been associated with intrinsic resistance to ICB in KRAS-mutant NSCLC (Koyama et al., 2016; Rizvi et al., 2018; Skoulidis et al., 2018); therefore, novel therapeutic approaches are needed to enhance immunogenicity. The adaptor protein STING, which links cytoplasmic dsDNA sensing by cGAS to activation of downstream innate immune signaling, is epigenetically silenced in KL cells (Kitajima et al., 2019). Accordingly, therapeutic restoration and activation of the STING pathway could represent a targeted approach to enhance immunogenicity in KL cells. In an unbiased screen of cytotoxic chemotherapies or targeted DNA-damaging agents in clinical trials, MPS1, a master-regulator of the SAC, was identified as a highly robust target to activate cGAS-STING signaling in KL cells. STING agonism induced by MPS1 inhibition is related to the ability to proceed through an abnormal mitosis and generate micronuclei, which are known potent activators of cGAS (Harding et al., 2017; Mackenzie et al., 2017; Mohr et al., 2021). Pulse treatment with MPS1 inhibitors following epigenetic inhibitors such as decitabine and/or GSK126 dramatically enhances STING pathway activation, leading to increased secretion of effector cytokines/chemokines such as CXCL10 and IFN-0, expression of MHC class I molecules, and direct STAT1-dependent cell death. Consistent with these results in vitro, combination therapy in a murine syngeneic KL model with the clinically relevant MPS1 inhibitor BAY-1217389 and decitabine strongly activated cancer cell-intrinsic STING and enhanced T cell recruitment in vivo, achieving prolonged preclinical activity over 12 weeks despite using a limited pulse treatment schedule.

A wide variety of synthetic STING agonists have been developed including cyclic dinucleotide (CDN) analogues based on the 2′3′-cGAMP structure as well as non-CDN molecules (Chin et al., 2020; Kwon and Bakhoum, 2020; Pan et al., 2020). In general, synthetic STING agonists target the STING pathway most potently in surrounding non-malignant cells, especially in myeloid cells to boost antitumor immunity via enhanced cross presentation of neoantigen and recruitment of cytotoxic T cells (Amouzegar et al., 2021). On the other hand, activity of the cancer cell-intrinsic STING pathway defines their immunogenicity and influences the efficacy of ICB. Indeed, beyond the KL cell state, loss of cGAS and/or STING is frequently observed in several types of cancer cells including melanoma and colorectal cancer, leading to escape from cancer immunosurveillance (Konno et al., 2018; Luo et al., 2020; Xia et al., 2016a; Xia et al., 2016b). Re-activation of STING signaling in STING-silenced cancers could be a direct approach to recover their immunogenicity and sensitize to ICB therapy. However, because synthetic STING agonists in general penetrate the cancer cell membrane poorly, an alternate approach was utilized to increase the production of KL cancer cell intrinsic 2′3′-cGAMP by stimulation of cGAS.

Micronuclei are discrete DNA aggregates separated from the primary nucleus and recognized by cGAS as abnormal cytoplasmic DNA (Zierhut and Funabiki, 2020). Because micronuclei are formed following continuous mitotic progression along with DNA damage (Harding et al., 2017), drugs accelerating formation of micronuclei have the potential to stimulate the STING pathway specifically in rapidly proliferating cancer cells. MPS1 is an essential SAC kinase that maintains the fidelity of chromosome segregation; it is critical for the recruitment of SAC proteins to unattached kinetochores and regulation of spindle fidelity upstream of the RZZ complex (Maciejowski et al., 2017). Since most cancer cells show rapid proliferation and chromosomal instability, they depend on the SAC to properly segregate their abnormal genome during mitosis. Thus, abrogation of the SAC by continuous MPS1 inhibition results in intolerable levels of genomic instability and cell death. Here, it has been demonstrated that transient treatment with an MPS1 inhibitor robustly generates micronuclei via chromosome missegregation in KL cells. In contrast to PARP inhibition, which is effective at activating STING in the context of synthetic lethal BRCA1/2 mutation (Ding et al., 2018; Pantelidou et al., 2019; Reislander et al., 2019), transient MPS1 inhibition may potentially engage the STING pathway in a variety of rapidly proliferating cancer cells that generate micronuclei and are capable of surviving long enough to promote an immunogenic TME. Importantly, continuous MPS1i, which favors mitotic arrest at the SAC, impaired cell proliferation and partially engaged cGAS/STING, but was not nearly as potent at activating this pathway as loading cells with micronuclei following pulse therapy. Indeed, transient MPS1 inhibition in an immunocompetent mouse model following decitabine induction therapy promoted remarkable tumor shrinkage and durable response compared with prior studies utilizing tumor cell xenografts in immunodeficient mice (Maia et al., 2018; Wengner et al., 2016). This was associated with reversal of the T-cell excluded phenotype, depletion of Tregs, and dependence on CD8+ T-cells, defining restoration of anti-tumor immunity as a key mediator of therapeutic activity.

Epigenetic regulation of innate immune signaling has become a major focus to promote cancer cell immunogenicity, as it represents a potentially reversible mechanism utilized by tumors to evade from immunosurveillance and ICB-mediated T cell killing (Topper et al., 2020). As observed with STING suppression in KL cells, genes regulating tumor cell recognition and type I interferon responses including TAP1/MHC class I, endogenous retroviruses (ERVs), and interferon-responsive genes more generally can be silenced by epigenetic mechanisms via DNA and histone lysine methylation (Canadas et al., 2018; Loo Yau et al., 2021; Mahadevan et al., 2021; Morel et al., 2021). Because cell division is required for the removal of functional epigenetic marks, DNA- or histone-demethylating agents might efficiently convert cell state from immunosuppressive to active in rapidly proliferating cancer cells compared with surrounding non-malignant cells, similar to the requirement of mitotic progression for micronuclei generation by MPS1 inhibition. Therefore, sequential epigenetic priming and pulse MPS1 inhibition could not only selectively targets anticancer immunity, but also minimizes the toxicity of inhibiting each target since drugs are not given simultaneously. Furthermore, systemic administration of this regimen impacts all tumor sites, in potential contrast to the limitations of injectable STING agonists or the potentially narrow therapeutic window of systemic STING agonists.

Finally, although mouse tumor models may not always predict durable response in humans, the ability of KL cancers to mask tumor antigens and likely avoid immune editing during tumor development may create a unique immune vulnerability. Tumor cell STING activation can also prime responses to T and NK cell therapy (Ji et al., 2021; Xu et al., 2021), and export of 2′-3′ cGAMP can also prime vascular activation for immune cell extravasation (Campisi et al., 2020). Thus, immunogenic priming of KL tumors by epigenetic therapy and MPS1 inhibition could also serve to facilitate tumor infiltration by engineered T and/or NK cell therapies. Translating this regimen into the clinic, restoring exposure of KL tumor antigens, and promoting effector cell recruitment, may have substantial potential to regenerate effective anti-tumor immunity for patients with treatment refractory KL tumors.

### Materials and Methods

### Cell Lines

A549, H2009, HEK293T, LLC, and CMT-167 cells were cultured in DMEM (Thermo Fisher Scientific, Cat. #11965-118) supplemented with 10% fetal bovine serum (FBS) (Gemini Bio-products, Cat. #100-106), 1× penicillin-streptomycin (Gemini Bio-products, Cat #400-109), and 2.5 μg/ml plasmocin prophylactic (Invivogen, Cat. #ant-mpp). H1944, H23, H1355, H647, H2122, A427, H1792, H441, H358, HCC44, THP-1, Jurkat, 393P, and Lacun3 cells were cultured in RPMI 1640 (Thermo Fisher Scientific, Cat. #11875-119) supplemented with 10% FBS, 1× penicillin-streptomycin, and 2.5 μg/ml plasmocin prophylactic (Invivogen, Cat. #ant-mpp). NK-92 cells were cultured in aMEM supplemented with 0.2 mM inositol, 0.1 mM 2-mercaptoethanol, 0.02 mM folic acid, 200 U/ml recombinant IL-2, 12.5% FBS, 12.5% horse serum, and 1× penicillin-streptomycin. HUVEC cells were cultured in vascular medium (VascuLife® VEGF Endothelial Medium Complete Kit, #LL-0003). A549, A427, H1944, H23, H1355, H2122, H1792 and H2009 cells were authenticated by short tandem repeat (STR) genotyping. DFCI-24, DFCI-298, DFCI-316, and DFCI-332 were established as described before (Kohler et al., 2021). DFCI-316 cells were grown in RPMI 1640 supplemented with 10% FBS, and 1× penicillin-streptomycin. DFCI-24, DFCI-298, and DFCI-332 were grown in ACL4 media supplemented with 10% FBS, and 1× penicillin-streptomycin. CD3+ T cells were isolated from PBMCs (STEMCELL, Cat. #70025) using EasySep™ Human T Cell Isolation Kit (STEMCELL, Cat. #70025) according to the manufacturer's instructions and cultured in RPMI 1640 supplemented with 10% human serum (Sigma-Aldrich, Cat. #H5667), 1× penicillin-streptomycin, 2 mM of L-Glutamine, and 100 IU/ml of IL-2, 25 ng/ml of IL-7, and 25 ng/ml of IL-15. The T cells were activated with 1% T cell TransAct (Miltenyi Biotec, Cat. #130-128-758) immediately after the isolation. A549, A427, H1944, H23, H1355, H2122, H1792 and H2009 cells were authenticated by short tandem repeat (STR) genotyping. HEK293T, H657, H441, H358, HCC44, THP-1, Jurkat, NK-92, and LLC were purchased from ATCC. CMT-167 cells were purchased from ECACC. HUVEC cells were purchased from Lonza (Lonza, C2519A). 393P cells were established from KrasLA1/+;p53R172HAG mice. Lacun3 cells were established from a chemically induced lung adenocarcinoma. All experiments were performed before reaching 10 passages from the original frozen stocks. Mycoplasma infection was regularly checked by MycoAlert™ Mycoplasma Detection Kit (Lonza, Cat. #LT07-218) according to the manufacturer's instructions.

### Reagents

The following reagents were used: 2′3′-cGAMP (Invivogene, Cat. #tlrl-nacga23), ADU-S100 (Chemietek, Cat. #CT-ADUS100), ruxolitinib (Selleckchem, Cat. #S1378), cisplatin (Sigma Aldrich, Cat. #232120), docetaxel (Selleckchem, Cat. #S1148), etoposide (Sigma Aldrich, Cat. #341205), vinorelbine (Sigma Aldrich, Cat. #V2264), pemetrexed (Selleckchem, Cat. #S1135), methotrexate (Sigma Aldrich, Cat. #A6770), aminopterin (Sigma Aldrich, Cat. #A1784), nocodazole (Sigma Aldrich, Cat. #M1404), hydroxyurea (Sigma Aldrich, Cat. #H8627), olaparib (Selleckchem, Cat. #S1060), barasertib (Cayman Chemical, Cat. #11602), MK5108 (Cayman Chemical, Cat. #19167), MK1775 (Cayman Chemical, Cat. #21266), ceralasertib (Cayman Chemical, Cat. #21035), prexasertib (Cayman Chemical, Cat. #21490), CFI-402257 (Cayman Chemical, Cat. #21960), volasertib (Cayman Chemical, Cat. #18193), BAY-1217389 (Selleckchem, Cat. #S8215), CC-671 (MedChemExpress, Cat. #HY-108709), IFNβ (R&D systems, Cat. #285-IF-100), decitabine (Selleckchem, Cat. #S1200), and GSK126 (Selleckchem, Cat. #S7061).

### ELISA

Human IFN-β (Thermo Fisher Scientific, Cat. #414101), human CXCL10 (R&D systems, Cat. #DIP100), mouse CXCL10 (R&D systems, Cat. #DY466), and 2′3′-cGAMP (Cayman Chemical, Cat. #501700) ELISAs were performed according to the manufacturer's instructions. Conditioned media from each cell lines was collected after 24 hour culture. Values represent the average of four replicates from at least two independent experiments (biological replicates).

### Generation of Lentivirus

3×10⁶ HEK293T cells were plated onto a 60-mm dish and transfected using X-tremeGENE HP DNA Transfection Reagent (Roche, Cat. #06366236001) with 1 μg of lentivirus-based expression vectors together with 1 μg of pCMV-dR8.91 and 1 μg of pCMV-VSV-G. After 48-hour incubation, the media containing lentivirus particles were collected, passed through a 0.45 μm filter, and concentrated using Lenti-X Concentrator (Clontech, Cat. #631231). For selection of virally infected cells, 1-2 μg/ml of puromycin (pCRISPR-v2 sgRNAs, plx307-hCXCR3) or 1.5-8 μg/ml of blasticidin (plx304-NanoLuc, plx304-hLKB1, plx304-STING, plx304-cGAS) was used 24 hours post-infection.

### Immunoblotting

Cells were lysed in RIPA buffer containing 1× protease inhibitors (Roche, Cat #11-836-145-001) and phosphatase inhibitors (50 mmol/L NaF and 100 mmol/L Na3VO4). Immunoblotting was performed as described (Kitajima et al., 2018) using the following antibodies to: cGAS (#15102, Cell Signaling Technology), STING (#13647, Cell Signaling Technology), STING (Rodent preferred) (#50494, Cell Signaling Technology), phospho-STAT1 (#9167, Cell Signaling Technology), STAT1 (#9172, Cell Signaling Technology), LKB1 (#3047, Cell Signaling Technology), cleaved PARP (#5625, Cell Signaling Technology), IFNAR1 (A304-290A, Thermo Fisher), phospho-Histone H2A.X (#9718, Cell Signaling Technology), Histone H3 (#4499, Cell Signaling Technology), phospho-TBK1 (#5483, Cell Signaling Technology), TBK1 (#3013, Cell Signaling Technology), DNMT1 (#5032, Cell Signaling Technology), Lamin B2 (#ab151735, Abcam), and j-Actin (#3700, Cell Signaling Technology). Secondary antibodies were from LICOR Biosciences: IRDye 680LT Goat anti-Mouse IgG (#926-68020), IRDye 800CW Goat anti-Rabbit IgG (#926-32211), or Cell Signaling Technology: anti-mouse IgG HRP-linked antibody (#7076), anti-rabbit IgG HRP-linked antibody (#7074). Imaging of blots and quantitation of bands was performed using the LICOR Odyssey system, or LAS-3000 (Fujifilm).

### CRSIPR/Cas9 System

Target sequences for CRISPR interference were designed using the sgRNA designer (portals.broadinstitute.org/gpp/public/analysis-tools/sgrna-design). A non-targeting sgRNA from the Gecko library v2 was used as a scramble sgRNA. sgRNA target sequences are listed in Table 2. sgRNAs were cloned into pCRISPRv2-puro.

TABLE 2
related to STAR Methods
Sequence for qRT-PCR primers and sgRNAs

| | | SEQ | | SEQ |
|---|---|---|---|---|
| | Forward | ID | Reverse | ID |
| | (5′-3′) | NO: | (5′-3′) | NO: |
| human | GTGGCATTCAA | 2 | TGATGGCCTTCGATT | 6 |
| CXCL10 | GGAGTACCTC | | CTGGATT | |
| human | CAGATTGGCTA | 3 | GGAAGGTGTAATCCG | 7 |
| 36B4 | CCCAACTGTT | | TCTCCA | |
| mouse | CCAAGTGCTGC | 4 | GGCTCGCAGGGATGA | 8 |
| CXCL10 | CGTCATTTTC | | TTTCAA | |
| mouse | AGATTCGGGAT | 5 | TCGGGTCCTAGACCA | 9 |
| 36B4 | ATGCTGTTGGC | | GTGTTC | |

| | | SEQ |
|---|---|---|
| | Target sequence | ID |
| | (5′-3′) | NO: |
| scramble sgRNA for | ATCGTTTCCGCTTAACGGCG | 10 |
| human | | |
| human LKB1 sgRNA | CTTCAAGGTGGACATCTGGT | 11 |
| human cGAS sgRNA | AAGTGCGACTCCGCGTTCAG | 12 |
| human STING sgRNA | GGTACCGGGGCAGCTACTGG | 13 |
| human IFNAR1 sgRNA | GATCTAATGTTAAAGACTGG | 14 |
| human STAT1 sgRNA | TGCTGGCACCAGAACGAATG | 15 |
| human ATG5 sgRNA | AAGAGTAAGTTATTTGACGT | 16 |
| scramble sgRNA for | ACGTGTAAGGCGAACGCCTT | 17 |
| mouse | | |
| mouse Sting sgRN | CAGTAGTCCAAGTTCGTGCG | 18 |
| mouse Lkb1 sgRNA | TCATCGGCAAGTACCTGATG | 19 |

dsDNA Stimulation

3×10⁵ cells were plated onto a 6-well plate and transfected using X-tremeGENE HP DNA Transfection Reagent (Roche, Cat. #06366236001) with the indicated amount of poly (dA:dT) (Invivogen, Cat. #tlrl-patn).

### Cell Viability Assay to Determine an IC50

3000 cells were plated onto 96-well plates, and then cultured for 72 hours in the presence of each DNA-damaging agent at the indicated concentration. Values of CellTiter-Glo Luminescent Cell Viability assay (Promega) after 96 hours were normalized to vehicle treated cells. Plates were read on a Tecan Infinite M200 Pro plate reader and analysis was performed using Prism7 (GraphPad Software). All conditions were tested in triplicate.

siRNA Transfection

siRNAs targeting MPS1 (s121), AURKB (s17611), and negative control (AM4611) were purchased from Thermo Fisher Scientific. Cells were transfected with 100 nM of the respective siRNAs using XtremeGENE siRNA transfection reagent (Roche, Cat. #4476093001) following the manufacturer's instructions, and collected after 96 hour culture.

### Quantification of Micronucleus Formation

Cells were plated onto chamber slides (CellTreat, Cat #229168), and treated with DNA-damaging agents for 48 hours. Cells were then cultured for 24 hours in normal growth medium after drug withdrawal, fixed in 4% paraformaldehyde (PFA, Electron Microscopy Sciences, Cat #15700) for 15 minutes at room temperature (RT), permeabilized with 0.1% vol/vol Triton X-100 for 5 minutes at RT, and stained with 1 μg/ml DAPI for 5 minutes at RT. Treated cells were imaged using an Olympus spinning disk confocal Imaging System (IX3-SPIN) equipped with a 60× silicon oil-immersion objective. Each image was taken with z-stack at 0.43 μm interval to cover the entire cells of interest. Z-stack images were subjected to maximum projection followed by quantitative analysis using CellSens. All samples were imaged and analyzed with the same setting throughout the experiments. The number of micronuclei were counted from three different fields for each sample. To examine co-localization of cGAS with micronuclei, cells were washed twice by PBS and fixed in 4% paraformaldehyde (PFA, Electron Microscopy Sciences, Cat #15700) for 15 minutes at RT. Cells were then permeabilized with 0.1% vol/vol Triton X-100 for 10 minutes at RT and washed twice by PBS. After blocking with 1% BSA (Sigma-Aldrich, cat #A4503-100G) in PBS for 1 hour at RT, cells were stained using the following antibody to: cGAS (#15102, Cell Signaling Technology). Secondary antibody was anti-rabbit AlexaFluor488 (1:1,000; Invitrogen Cat. #A11034). Cells were then subjected to 5 minutes of DAPI (D9542, Sigma-Aldrich) staining and washed twice by PBS before cover slides were mounted with Vectashield hardset mounting medium (H-1400-10, Vector Laboratories). Slides were imaged with Zeiss LSM 880 confocal microscopy, and colocalization was determined using ImageJ.

### Quantitative RT-PCR

RNA extraction was performed using RNeasy Mini Kit (Qiagen, Cat. #74106). RNA samples (1 μg) were reverse-transcribed using SuperScript® III First-Strand Synthesis SuperMix (Thermo Fisher Scientific, Cat. #1683483). Quantitative real-time PCR was performed using Power SYBR Green PCR Master Mix (Thermo Fisher Scientific, Cat. #4367659). The sequences of the primers used for qRT-PCR are listed in Table 2. Values represent the average of four technical replicates from at least two independent experiments (biological replicates).

### Analysis of Cell Cycle and Cell Viability

For cell cycle analysis, cells were stained by BD CycleTEST™ Plus DNA according to the manufacturer's instructions, and then analyzed by FACSCanto 11 (BD Biosciences). For cell viability analysis, cells were stained by propidium iodide (PI) according to the manufacturer's instructions of Annexin V using Alexa Fluor 488 Annexin V dead cell apoptosis kit (Thermo Fisher Scientific, Cat. #V13245) and then analyzed by FACSCanto 11 (BD Biosciences).

### Immunofluorescence Staining

After being grown on chamber slides (CellTreat, Cat #229168) and subjected to various treatment conditions, cells were fixed and permeabilized according to standard protocols. In brief, cells were washed twice by PBS and fixed in 4% paraformaldehyde (PFA, Electron Microscopy Sciences, Cat #15700) for 15 minutes at RT. Cells were then permeabilized with 0.1% vol/vol Triton X-100 for 10 minutes at RT and washed twice by PBS. After blocking with 1% BSA (Sigma-Aldrich, cat #A4503-100G) in PBS for 1 hour at RT, cells were stained using the following antibodies to: STING (PA5-23381, Invitrogen) and ERGIC53 (PSC-PM-7213-C100, Axxora). Secondary antibodies were anti-mouse AlexaFluor488 (1:1,000; Invitrogen Cat. #A21202), or anti-rabbit AlexaFluor555 (1:1,000; Invitrogen Cat. #A21428). Cells were then subjected to 20 minutes of DAPI (D9542, Sigma-Aldrich) staining and washed twice by PBS before cover slides were mounted with Vectashield hardset mounting medium (H-1400-10, Vector Laboratories). Slides were imaged with the Nikon Eclipse 80i microscope, and colocalization was determined using CoLoc2 in ImageJ.

### Immune Profiling by Flow Cytometry

Fresh tumor tissue was placed in dissociation buffer consisting of RPMI (Life Technologies, Carlsbad, CA)+10% FBS (HyClone, Logan, UT), 100 U/mL collagenase type IV (Life Technologies, Carlsbad, CA), and 50 μg/mL DNase I (Roche, Indianapolis, IN) at a ratio of 5 mL of dissociation buffer per 500 mg of sample and mechanically separated using gentleMACS C Tubes and gentleMACS Octo Dissociator system according to the manufacturer's protocol (Miltenyi, San Diego, CA). Suspension was incubated at 37° C. for 45 minutes. Red blood cells were removed from samples using red blood cell lysis buffer (BioLegend, San Diego, CA). Samples were pelleted and then resuspended in fresh RPMI+10% FBS and strained over a 70 μm filter. Cells were incubated with Live/Dead Fixable Zombie NIR™ (Biolegend, San Diego, CA) for 5 minutes in the dark at room temperature in PBS. Fc receptors were blocked prior to surface antibody staining using mouse TruStain FcX blocking reagent (Biolegend, San Diego, CA). Cells were stained with pre-conjugated antibodies for 15 minutes on ice in FBS+2% FBS and washed prior to analysis on a BD LSRFortessa with FACSDiva software (BD Biosciences, San Jose, CA). Data were analyzed using FlowJo (Ashland, OR) software version 10.7.1. Antibodies were specific for the following mouse markers: CD3 (17A2), CD4 (GK1.5), CD8 (53-6.7), CD11b (M1/70), CD19 (6D5), CD25 (PC61), CD45 (30-F11), CD49b (DX5), Ly6G (1A8), LAG-3 (C9B7W), PD-1 (29F.1A12), and TIM-3 (B8.2C12), all from Biolegend (San Diego, CA), and Foxp3 (FJK-16s) from Thermo Fisher Scientific. To analyze cell surface markers in vitro, 2×10⁵ cells resuspended in 100 μl PBS containing 3% FBS were stained by FITC-conjugated anti-CXCR3 antibody (Biolegend, Cat. #353703), FITC-conjugated anti-HLA-A.B.C antibody (Biolegend, Cat. #311404), or PerCP/Cy5.5-conjugated anti-PD-L1 antibody (Biolegend, Cat. #329738) for 30 minutes at room temperature, washed by PBS containing 3% FBS, and then analyzed by FACSCanto 11 (BD Biosciences) or FACSLyric (BD Biosciences). FITC-conjugated mouse IgG2a (Biolegend, Cat. #400208) or PerCP/Cy5.5-conjugated mouse IgG2b (Biolegend, Cat. #400338) was used as isotype control antibody.

### Immune Cell Migration Assay

Immune cell migration assay was performed as previously described (Kitajima et al., 2019; Ritter et al., 2020). Briefly, cancer cell spheroids (h1944) were generated by seeding 5×10⁵ cells in suspension in an ultra-low attachment dish (Corning, Cat. #3471) for 24 hours. Samples were pelleted and then resuspended in type I rat tail collagen (Corning) at a concentration of 2.5 mg/mL following the addition of 10×PBS with phenol red with pH adjusted using NaOH. pH 7.0-7.5 was confirmed using PANPEHA Whatman paper (Sigma-Aldrich). Cells and collagen are kept on ice. The spheroids-collagen suspension was then injected into the central gel region of the 3D DAX-1 3-D microfluidic cell culture chip (AIM Biotech, Singapore, Cat. #DAX-1). Microfluidic devices were designed as previously described (Aref et al., 2018), with a central region containing the cell-collagen mixture in a 3D microenvironment, surrounded by 2 media channels located on either side. After injection, collagen hydrogels containing cells were incubated 40 minutes at 37° C. in humidity chambers, then hydrated with culture media, with 5×10⁴ CXCR3-overexpressing Jurkat cells in one of the side media channels. CXCR3-overexpressing Jurkat cells were labeled with Cell Tracker Red (Thermo Fisher Scientific, Cat. #C34552) following the manufacturer's instructions. After 72-96 hours of incubation, cancer cell spheroids and infiltrated immune cells were stained for 15 minutes with Acridine orange (AO) diluted 1:1 in culture media, (ViaStain™ AO Staining Solution—CS1-0108-5 mL, nexcelom). For NK cell migrations, NK-92 cells (ATCC) were cultured as previously described. 5×10⁴ cells were stained with cell Blue dye (cell proliferation dye eFluor 450, Invitrogen, Cat. #65-0842) and cultured in the device for 3 days with IL-2 deprivation, followed by culture in the device with a full complete media for a total of 144 hours. For PBMC-derived CD3+ T cells migrations, 5×10⁴ were stained with cell Blue dye and cultured in the device with RPMI 1640 supplemented with 10% human serum, 2 mM of L-Glutamine, 1× penicillin-streptomycin, and 100 IU/ml of IL-2, 25 ng/ml of IL-7, and 25 ng/ml of IL-15 for a total of 72 hours.

Co-Culture Patient-Derived Tumor Cells with PBMC-Derived T Cell

1×10⁵ cancer cells/well and PBMC derived 1×105 CD3+ T cells/well were seeded in 96-well plates. Twenty-four hours after seeding the cells, conditioned media from each well were collected, and human Granzyme B (R&D systems, Cat. #DGZB00) ELISA was performed according to the manufacturer's instructions.

### Cytokine Profiling

Multiplex assays were performed utilizing the Mouse Cytokine/Chemokine Magnetic Bead Panel (Cat. #MCYTMAG-70K-PX32) on a Luminex MAGPIX system (Merck Millipore). Fold changes relative to the corresponding control were calculated and plotted as log₂FC. Lower and upper limits of quantitation (LLOQ/ULOQ) were imputed from standard curves for cytokines above or below detection.

### IHC Staining and Analysis

Immunohistochemistry was performed on the Leica Bond III automated staining platform. The antibody for CD3F (Cell Signaling Technology #99940, clone D4V8L) was run at 1:150 dilution using the Leica Biosystems Refine Detection Kit with EDTA antigen. The antibody for CD8a (Cell Signaling Technology #98941, clone D4W2Z) was run at 1:200 dilution using the Leica Biosystems Refine Detection Kit with EDTA antigen. CD3 IHC staining was quantified using QuPath software (0.2.0-m4) (Bankhead et al., 2017). Positive Pixel Detection analysis was used with default settings for DAB staining to detect and quantify positive pixels in each of three individual, randomly selected fields from the center of each mouse tumor.

### Animal Study

All mouse experiments were conducted according to a Dana-Farber Cancer Institute approved protocol. Five million cells (393P-KL, 393P-STING KO) in PBS with 30% Matrigel (Coming, NY) were subcutaneously injected into the flank of 8-week-old 129-Elite mice (129S2/SvPasCrl, Strain code 476, Charles River Laboratories) or NSG (NOD.Cg-Prkdcscid Il2rgtm1Wjl/SzJ) mice (The Jackson Laboratory, ME). Tumor volume was determined from caliper measurements of tumor length (L) and width (W) according to the formula (L×W2)/2. Animals were randomized (Studylog software, CA) into various treatment groups once tumor volumes were in the range of 110-190 mm³ for efficacy studies and in the range of 280-410 mm³ for the PD study before treatment initiation. The durable response was defined as mice with tumor volume less than 250 mm³ for at least 50 days after treatment was completed. Both tumor size and body weight were measured twice per week. BAY-1217389 was formulated in 50% PEG 400, 10% ethanol and 40% water and dosed at 5 mg/kg twice daily by oral gavage. Decitabine was dissolved in saline and dosed at 0.5 mg/kg once per day by intraperitoneal injection. For CD8 depletion study, tumor bearing mice were injected intraperitoneally with CD8+ T cell depleting antibody (Clone 53-6.7 from BioXcell, NH) diluted in PBS at a concentration of 250 μg/mouse. In satellite animals, spleen and tumor tissue was isolated 24 hours after the last treatment and subjected to FACS analysis.

### Statistical Analysis

Statistical significance was assessed using unpaired two-tailed Student's t-test, one-way ANOVA followed by Tukey's post-hoc test or by Dunnet's post-hoc test, or two-way ANOVA followed by Sidak's post-hoc test. p values less than 0.05 were considered significant. Asterisks used to indicate significance correspond with: *p<0.05, **p<0.01. Columns represent means±standard deviation (S.D.). Mouse tumor volume data with means±standard error (S.E.) is shown. In one-way or two-way ANOVA followed by post-hoc tests, asterisks are shown only in pairs of interest. GraphPad Prism7 was used for all statistical analysis.

### REFERENCES

Amouzegar, A., Chelvanambi, M., Filderman, J. N., Storkus, W. J., and Luke, J. J. (2021). STING Agonists as Cancer Therapeutics. Cancers (Basel) 13.
Aref, A. R., Campisi, M., Ivanova, E., Portell, A., Larios, D., Piel, B. P., Mathur, N., Zhou, C., Coakley, R. V., Bartels, A., et al. (2018). 3D microfluidic ex vivo culture of organotypic tumor spheroids to model immune checkpoint blockade. Lab Chip 18, 3129-3143.
Bankhead, P., Loughrey, M. B., Fernandez, J. A., Dombrowski, Y., McArt, D. G., Dunne, P. D., McQuaid, S., Gray, R. T., Murray, L. J., Coleman, H. G., et al. (2017). QuPath: Open source software for digital pathology image analysis. Sci Rep 7, 16878.
Campisi, M., Sundararaman, S. K., Shelton, S. E., Knelson, E. H., Mahadevan, N. R., Yoshida, R., Tani, T., Ivanova, E., Canadas, I., Osaki, T., et al. (2020). Tumor-Derived cGAMP Regulates Activation of the Vasculature. Front Immunol 11, 2090.
Canadas, I., Thummalapalli, R., Kim, J. W., Kitajima, S., Jenkins, R. W., Christensen, C. L., Campisi, M., Kuang, Y., Zhang, Y., Gjini, E., et al. (2018). Tumor innate immunity primed by specific interferon-stimulated endogenous retroviruses. Nat Med 24, 1143-1150.
Chin, E. N., Yu, C., Vartabedian, V. F., Jia, Y., Kumar, M., Gamo, A. M., Vernier, W., Ali, S. H., Kissai, M., Lazar, D. C., et al. (2020). Antitumor activity of a systemic STING-activating non-nucleotide cGAMP mimetic. Science 369, 993-999.
Cordova, A. F., Ritchie, C., Bohnert, V., and Li, L. (2021). Human SLC46A2 Is the Dominant cGAMP Importer in Extracellular cGAMP-Sensing Macrophages and Monocytes. ACS Cent Sci 7, 1073-1088.
Dai, J., Huang, Y. J., He, X., Zhao, M., Wang, X., Liu, Z. S., Xue, W., Cai, H., Zhan, X. Y., Huang, S. Y., et al. (2019). Acetylation Blocks cGAS Activity and Inhibits Self-DNA-Induced Autoimmunity. Cell 176, 1447-1460 e1414.
Deng, J., Thennavan, A., Dolgalev, I., Chen, T., Li, J., Marzio, A., Poirier, J. T., Peng, D., Bulatovic, M., Mukhopadhyay, S., et al. (2021). ULK1 inhibition overcomes compromised antigen presentation and restores antitumor immunity in LKB1 mutant lung cancer. Nat Cancer 2, 503-514.
Deng, L., Liang, H., Xu, M., Yang, X., Burnette, B., Arina, A., Li, X. D., Mauceri, H., Beckett, M., Darga, T., et al. (2014). STING-Dependent Cytosolic DNA Sensing Promotes Radiation-Induced Type I Interferon-Dependent Antitumor Immunity in Immunogenic Tumors. Immunity 41, 843-852.
Ding, L., Kim, H. J., Wang, Q., Kearns, M., Jiang, T., Ohlson, C. E., Li, B. B., Xie, S., Liu, J. F., Stover, E. H., et al. (2018). PARP Inhibition Elicits STING-Dependent Antitumor Immunity in Brcal-Deficient Ovarian Cancer. Cell Rep 25, 2972-2980 e2975.
Falahat, R., Berglund, A., Putney, R. M., Perez-Villarroel, P., Aoyama, S., Pilon-Thomas, S., Barber, G. N., and Mule, J. J. (2021). Epigenetic reprogramming of tumor cell-intrinsic STING function sculpts antigenicity and T cell recognition of melanoma. Proc Natl Acad Sci USA 118.
Falahat, R., Perez-Villarroel, P., Mailloux, A. W., Zhu, G., Pilon-Thomas, S., Barber, G. N., and Mule, J. J. (2019). STING Signaling in Melanoma Cells Shapes Antigenicity and Can Promote Antitumor T-cell Activity. Cancer Immunol Res 7, 1837-1848.
Gangaplara, A., Martens, C., Dahlstrom, E., Metidji, A., Gokhale, A. S., Glass, D. D., Lopez-Ocasio, M., Baur, R., Kanakabandi, K., Porcella, S. F., and Shevach, E. M. (2018). Type I interferon signaling attenuates regulatory T cell function in viral infection and in the tumor microenvironment. PLoS Pathog 14, e1006985.
Ghosh, M., Saha, S., Bettke, J., Nagar, R., Parrales, A., Iwakuma, T., van der Velden, A. W. M., and Martinez, L. A. (2021). Mutant p53 suppresses innate immune signaling to promote tumorigenesis. Cancer Cell 39, 494-508 e495.
Grabosch, S., Bulatovic, M., Zeng, F., Ma, T., Zhang, L., Ross, M., Brozick, J., Fang, Y., Tseng, G., Kim, E., et al. (2019). Cisplatin-induced immune modulation in ovarian cancer mouse models with distinct inflammation profiles. Oncogene 38, 2380-2393.
Guan, J., Lu, C., Jin, Q., Lu, H., Chen, X., Tian, L., Zhang, Y., Ortega, J., Zhang, J., Siteni, S., et al. (2021). MLH1 Deficiency-Triggered DNA Hyperexcision by Exonuclease 1 Activates the cGAS-STING Pathway. Cancer Cell 39, 109-121 e105.
Gulen, M. F., Koch, U., Haag, S. M., Schuler, F., Apetoh, L., Villunger, A., Radtke, F., and Ablasser, A. (2017). Signalling strength determines proapoptotic functions of STING. Nat Commun 8, 427.
Harding, S. M., Benci, J. L., Irianto, J., Discher, D. E., Minn, A. J., and Greenberg, R. A. (2017). Mitotic progression following DNA damage enables pattern recognition within micronuclei. Nature 548, 466-470.
Hatch, E. M., Fischer, A. H., Deerinck, T. J., and Hetzer, M. W. (2013). Catastrophic nuclear envelope collapse in cancer cell micronuclei. Cell 154, 47-60.
Hopfner, K. P., and Hornung, V. (2020). Molecular mechanisms and cellular functions of cGAS-STING signalling. Nat Rev Mol Cell Biol 21, 501-521.
Keenan, T. E., Burke, K. P., and Van Allen, E. M. (2019). Genomic correlates of response to immune checkpoint blockade. Nat Med 25, 389-402.
Kitajima, S., Asahina, H., Chen, T., Guo, S., Quiceno, L. G., Cavanaugh, J. D., Merlino, A. A., Tange, S., Terai, H., Kim, J. W., et al. (2018). Overcoming Resistance to Dual Innate Immune and MEK Inhibition Downstream of KRAS. Cancer Cell 34, 439-452 e436.
Kitajima, S., Ivanova, E., Guo, S., Yoshida, R., Campisi, M., Sundararaman, S. K., Tange, S., Mitsuishi, Y., Thai, T. C., Masuda, S., et al. (2019). Suppression of STING Associated with LKB1 Loss in KRAS-Driven Lung Cancer. Cancer Discov 9, 34-45.
Kohler, J., Zhao, Y., Li, J., Gokhale, P. C., Tiv, H. L., Knott, A. R., Wilkens, M. K., Soroko, K. M., Lin, M., Ambrogio, C., et al. (2021). ERK Inhibitor LY3214996-Based Treatment Strategies for RAS-Driven Lung Cancer. Mol Cancer Ther 20, 641-654.
Konno, H., Yamauchi, S., Berglund, A., Putney, R. M., Mule, J. J., and Barber, G. N. (2018). Suppression of STING signaling through epigenetic silencing and missense mutation impedes DNA damage mediated cytokine production. Oncogene 37, 2037-2051.
Koyama, S., Akbay, E. A., Li, Y. Y., Aref, A. R., Skoulidis, F., Herter-Sprie, G. S., Buczkowski, K. A., Liu, Y., Awad, M. M., Denning, W. L., et al. (2016). STK11/LKB1 Deficiency Promotes Neutrophil Recruitment and Proinflammatory Cytokine Production to Suppress T-cell Activity in the Lung Tumor Microenvironment. Cancer Res 76, 999-1008.
Kwon, J., and Bakhoum, S. F. (2020). The Cytosolic DNA-Sensing cGAS-STING Pathway in Cancer. Cancer Discov 10, 26-39.
Li, T., and Chen, Z. J. (2018). The cGAS-cGAMP-STING pathway connects DNA damage to inflammation, senescence, and cancer. J Exp Med 215, 1287-1299.
London, N., and Biggins, S. (2014). Signalling dynamics in the spindle checkpoint response. Nat Rev Mol Cell Biol 15, 736-747.
Loo Yau, H., Bell, E., Ettayebi, I., de Almeida, F. C., Boukhaled, G. M., Shen, S. Y., Allard, D., Morancho, B., Marhon, S. A., Ishak, C. A., et al. (2021). DNA hypomethylating agents increase activation and cytolytic activity of CD8(+) T cells. Mol Cell 81, 1469-1483 e1468.
Lu, C., Guan, J., Lu, S., Jin, Q., Rousseau, B., Lu, T., Stephens, D., Zhang, H., Zhu, J., Yang, M., et al. (2021). DNA Sensing in Mismatch Repair-Deficient Tumor Cells Is Essential for Anti-tumor Immunity. Cancer Cell 39, 96-108 e106.
Luo, X., Donnelly, C. R., Gong, W., Heath, B. R., Hao, Y., Donnelly, L. A., Moghbeli, T., Tan, Y. S., Lin, X., Bellile, E., et al. (2020). HPV16 drives cancer immune escape via NLRX1-mediated degradation of STING. J Clin Invest 130, 1635-1652.
Luteijn, R. D., Zaver, S. A., Gowen, B. G., Wyman, S. K., Garelis, N. E., Onia, L., McWhirter, S. M., Katibah, G. E., Corn, J. E., Woodward, J. J., and Raulet, D. H. (2019). SLC19A1 transports immunoreactive cyclic dinucleotides. Nature 573, 434-438.
Maciejowski, J., Drechsler, H., Grundner-Culemann, K., Ballister, E. R., Rodriguez-Rodriguez, J. A., Rodriguez-Bravo, V., Jones, M. J. K., Foley, E., Lampson, M. A., Daub, H., et al. (2017). Mps1 Regulates Kinetochore-Microtubule Attachment Stability via the Ska Complex to Ensure Error-Free Chromosome Segregation. Dev Cell 41, 143-156 e146.
Mackenzie, K. J., Carroll, P., Martin, C. A., Murina, O., Fluteau, A., Simpson, D. J., Olova, N., Sutcliffe, H., Rainger, J. K., Leitch, A., et al. (2017). cGAS surveillance of micronuclei links genome instability to innate immunity. Nature 548, 461-465.
Mahadevan, N. R., Knelson, E. H., Wolff, J. O., Vajdi, A., Saigi, M., Campisi, M., Hong, D., Thai, T. C., Piel, B., Han, S., et al. (2021). Intrinsic Immunogenicity of Small Cell Lung Carcinoma Revealed by Its Cellular Plasticity. Cancer Discov.
Maia, A. R. R., Linder, S., Song, J. Y., Vaarting, C., Boon, U., Pritchard, C. E. J., Velds, A., Huijbers, I. J., van Tellingen, O., Jonkers, J., and Medema, R. H. (2018). Mps1 inhibitors synergise with low doses of taxanes in promoting tumour cell death by enhancement of errors in cell division. Br J Cancer 118, 1586-1595.
Mason, J. M., Wei, X., Fletcher, G. C., Kiarash, R., Brokx, R., Hodgson, R., Beletskaya, I., Bray, M. R., and Mak, T. W. (2017). Functional characterization of CFI-402257, a potent and selective Mps1/TTK kinase inhibitor, for the treatment of cancer. Proc Natl Acad Sci U SA 114, 3127-3132.
Mohr, L., Toufektchan, E., von Morgen, P., Chu, K., Kapoor, A., and Maciejowski, J. (2021). ER-directed TREX1 limits cGAS activation at micronuclei. Mol Cell 81, 724-738 e729.
Morel, K. L., Sheahan, A. V., Burkhart, D. L., Baca, S. C., Boufaied, N., Liu, Y., Qiu, X., Canadas, I., Roehle, K., Heckler, M., et al. (2021). EZH2 inhibition activates a dsRNA-STING-interferon stress axis that potentiates response to PD-1 checkpoint blockade in prostate cancer. Nat Cancer 2, 444-456.
Olagnier, D., Brandtoft, A. M., Gunderstofte, C., Villadsen, N. L., Krapp, C., Thielke, A. L., Laustsen, A., Peri, S., Hansen, A. L., Bonefeld, L., et al. (2018). Nrf2 negatively regulates STING indicating a link between antiviral sensing and metabolic reprogramming. Nat Commun 9, 3506.
Pan, B. S., Perera, S. A., Piesvaux, J. A., Presland, J. P., Schroeder, G. K., Cumming, J. N., Trotter, B. W., Altman, M. D., Buevich, A. V., Cash, B., et al. (2020). An orally available non-nucleotide STING agonist with antitumor activity. Science 369.
Pantelidou, C., Sonzogni, O., De Oliveria Taveira, M., Mehta, A. K., Kothari, A., Wang, D., Visal, T., Li, M. K., Pinto, J., Castrillon, J. A., et al. (2019). PARP Inhibitor Efficacy Depends on CD8(+) T-cell Recruitment via Intratumoral STING Pathway Activation in BRCA-Deficient Models of Triple-Negative Breast Cancer. Cancer Discov 9, 722-737.
Reislander, T., Groelly, F. J., and Tarsounas, M. (2020). DNA Damage and Cancer Immunotherapy: A STING in the Tale. Mol Cell 80, 21-28.
Reislander, T., Lombardi, E. P., Groelly, F. J., Miar, A., Porru, M., Di Vito, S., Wright, B., Lockstone, H., Biroccio, A., Harris, A., et al. (2019). BRCA2 abrogation triggers innate immune responses potentiated by treatment with PARP inhibitors. Nat Commun 10, 3143.
Ricciuti, B., Arbour, K. C., Lin, J. J., Vajdi, A., Vokes, N., Hong, L., Zhang, J., Tolstorukov, M. Y., Li, Y. Y., Spurr, L. F., et al. (2022). Diminished Efficacy of Programmed Death-(Ligand)1 Inhibition in STK11—and KEAP1-Mutant Lung Adenocarcinoma Is Affected by KRAS Mutation Status. J Thorac Oncol 17, 399-410.
Ritchie, C., Cordova, A. F., Hess, G. T., Bassik, M. C., and Li, L. (2019). SLC19A1 Is an Importer of the Immunotransmitter cGAMP. Mol Cell 75, 372-381 e375.
Ritter, J. L., Zhu, Z., Thai, T. C., Mahadevan, N. R., Mertins, P., Knelson, E. H., Piel, B. P., Han, S., Jaffe, J. D., Carr, S. A., et al. (2020). Phosphorylation of RAB7 by TBK1/IKKepsilon Regulates Innate Immune Signaling in Triple-Negative Breast Cancer. Cancer Res 80, 44-56.
Rizvi, H., Sanchez-Vega, F., La, K., Chatila, W., Jonsson, P., Halpenny, D., Plodkowski, A., Long, N., Sauter, J. L., Rekhtman, N., et al. (2018). Molecular Determinants of Response to Anti-Programmed Cell Death (PD)-1 and Anti-Programmed Death-Ligand 1 (PD-L1) Blockade in Patients With Non-Small-Cell Lung Cancer Profiled With Targeted Next-Generation Sequencing. J Clin Oncol 36, 633-641.
Skoulidis, F., Goldberg, M. E., Greenawalt, D. M., Hellmann, M. D., Awad, M. M., Gainor, J. F., Schrock, A. B., Hartmaier, R. J., Trabucco, S. E., Gay, L., et al. (2018). STK11/LKB1 Mutations and PD-1 Inhibitor Resistance in KRAS-Mutant Lung Adenocarcinoma. Cancer Discov.
Spranger, S., and Gajewski, T. F. (2018). Impact of oncogenic pathways on evasion of antitumour immune responses. Nat Rev Cancer 18, 139-147.
Topper, M. J., Vaz, M., Marrone, K. A., Brahmer, J. R., and Baylin, S. B. (2020). The emerging role of epigenetic therapeutics in immuno-oncology. Nat Rev Clin Oncol 17, 75-90.
Wengner, A. M., Siemeister, G., Koppitz, M., Schulze, V., Kosemund, D., Klar, U., Stoeckigt, D., Neuhaus, R., Lienau, P., Bader, B., et al. (2016). Novel Mps1 Kinase Inhibitors with Potent Antitumor Activity. Mol Cancer Ther 15, 583-592.
Woo, S. R., Fuertes, M. B., Corrales, L., Spranger, S., Furdyna, M. J., Leung, M. Y., Duggan, R., Wang, Y., Barber, G. N., Fitzgerald, K. A., et al. (2014). STING-dependent cytosolic DNA sensing mediates innate immune recognition of immunogenic tumors. Immunity 41, 830-842.
Xia, T., Konno, H., Ahn, J., and Barber, G. N. (2016a). Deregulation of STING Signaling in Colorectal Carcinoma Constrains DNA Damage Responses and Correlates With Tumorigenesis. Cell Rep 14, 282-297.
Xia, T., Konno, H., and Barber, G. N. (2016b). Recurrent Loss of STING Signaling in Melanoma Correlates with Susceptibility to Viral Oncolysis. Cancer Res 76, 6747-6759.
Zhang, X., Bai, X. C., and Chen, Z. J. (2020). Structures and Mechanisms in the cGAS-STING Innate Immunity Pathway. Immunity 53, 43-53.
Zhao, M., Wang, F., Wu, J., Cheng, Y., Cao, Y., Wu, X., Ma, M., Tang, F., Liu, Z., Liu, H., and Ge, B. (2021). CGAS is a micronucleophagy receptor for the clearance of micronuclei. Autophagy 17, 3976-3991.
Zierhut, C., and Funabiki, H. (2020). Regulation and Consequences of cGAS Activation by Self-DNA. Trends Cell Biol 30, 594-605.
Zierhut, C., Yamaguchi, N., Paredes, M., Luo, J. D., Carroll, T., and Funabiki, H. (2019). The Cytoplasmic DNA Sensor cGAS Promotes Mitotic Cell Death. Cell 178, 302-315 e323.

### Other Embodiments

All the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method of treating a subject having cancer, comprising:
(i) administering to the subject a therapeutically effective amount of an epigenetic inhibitor; and
(ii) administering to the subject a therapeutically effective amount of a DNA damaging agent.

2. The method of claim 1, wherein the epigenetic inhibitor increases or restores expression of STING.

3. The method of claim 1, wherein the epigenetic inhibitor inhibits DNA methylation, histone methylation, or histone deacetylation.

4. The method of claim 1, wherein the epigenetic inhibitor comprises a DMNT1 inhibitor, an EZH2 inhibitor, or an HDAC inhibitor.

5. (canceled)

6. The method of claim 1, wherein the epigenetic inhibitor comprises decitabine.

7. The method of claim 1, wherein the DNA damaging agent comprises an agent that induces the formation of micronuclei.

8. The method of claim 1, wherein the DNA damaging agent comprises an MPS1 inhibitor or an anti-folate drug.

9. (canceled)

10. The method of claim 1, wherein the DNA damaging agent comprises BAY-1217389.

11. The method of claim 1, wherein the cancer is a lung cancer.

12-14. (canceled)

15. The method of claim 1, wherein the epigenetic inhibitor is administered to the subject for about 3, about 4, about 5, about 6, about 7, about 8, about 9, about 10, about 11, or about 12 or more days.

16. (canceled)

17. The method of claim 1, wherein the DNA damaging agent is administered to the subject for about 1, about 2, about 3, or about 4 or more days.

18. (canceled)

19. The method of claim 1 further comprising administering a therapeutically effective amount of the DNA damaging agent to the subject an additional time.

20. (canceled)

21. A method of treating cancer in a subject, comprising administering the treatment, when a biological sample from the subject has low levels of STING (stimulator of IFN genes) expression, high levels of expression of an epigenetic regulatory enzyme, or both low levels of STING expression and high levels of expression of an epigenetic regulatory enzyme;
wherein the treatment includes;
(i) administering to the subject a therapeutically effective amount of an epigenetic inhibitor; and
(ii) administering to the subject a therapeutically effective amount of a DNA damaging agent.

22. (canceled)

23. The method of claim 21, wherein the epigenetic inhibitor increases or restores expression of STING.

24. (canceled)

25. The method of claim 21, wherein the epigenetic inhibitor comprises a DMNT1 inhibitor, an EZH2 inhibitor, or an HD AC inhibitor.

26-28. (canceled)

29. The method of claim 21, wherein the DNA damaging agent comprises an MPS1 inhibitor or an anti-folate drug.

30-31. (canceled)

32. The method of claim 21, wherein the cancer is a lung cancer.

33-41. (canceled)

42. A kit comprising an epigenetic inhibitor and a DNA damaging agent.

43-52. (canceled)

53. The method of claim 1, wherein the DNA damaging agent is administered to the subject using a pulse treatment strategy.
